(19) United States
(12) Patent Application Publication
(10) Pub. No.: US 2025/0276070 A1
(43) Pub. Date: Sep. 4, 2025

(54) OPTIMIZED MULTABODY CONSTRUCTS, COMPOSITIONS, AND METHODS

(71) Applicants: The Hospital for Sick Children, Toronto (CA); THE GOVERNING COUNCIL OF THE UNIVERSITY OF TORONTO, Toronto (CA)

(72) Inventors: Jean-Philippe Julien, Toronto (CA); Edurne Rujas Diez, Vitoria (ES); Bebhinn Treanor, Toronto (CA); Tian Tian Zhao, Toronto (CA)

(21) Appl. No.: 18/578,239

(22) PCT Filed: Jul. 12, 2022
(86) PCT No.: PCT/CA2022/051084

§ 371 Date: Jan. 10, 2024

Related U.S. Application Data
(60) Provisional application No. 63/220,920, filed on Jul. 12, 2021.
(60) Provisional application No. 63/289,016, filed on Dec. 13, 2021.

(51) Int. Cl. A61K 47/64; A61K 47/68; C07K 16/46

(52) U.S. Cl. CPC A61K 47/64; A61K 47/68; C07K 16/46; C07K 2317/71; C07K 2319/00

(57) ABSTRACT

In aspects, a self-assembled polypeptide complex comprises (a) a plurality of first fusion polypeptides, each first fusion polypeptide comprising (1) an Fc polypeptide linked to (2) a nanocage monomer or subunit thereof, wherein the Fc polypeptide comprises a Fc chain having one or more mutations relative to a reference Fc chain of the same Ig class, and (b) a plurality of second fusion polypeptides, each second fusion polypeptide comprising (1) an antigen-binding antibody fragment linked to (2) a nanocage monomer or subunit thereof.

### CROSS-REFERENCE TO RELATED APPLICATIONS

The present application is a U.S. National Stage Application of International Patent Application No. PCT/CA2022/051084, filed Jul. 12, 2022, which claims the benefit of and priority to U.S. Provisional Patent Application Nos. 63/220,920, filed Jul. 12, 2021, and 63/289,016, filed Dec. 13, 2021, the entire contents of each of which are hereby incorporated by reference in their entirety for all purposes.

### SEQUENCE LISTING

The instant application contains a Sequence Listing which has been submitted electronically in XML format and is hereby incorporated by reference in its entirety. Said XML copy, created on Jul. 8, 2024, is named “RBT-006WOUS_SL.xml” and is 64,353 bytes in size.

### BACKGROUND

Therapeutics based on antibodies or antibody fragments are being developed for a variety of uses, e.g., for treating various diseases or conditions.

However, antibody-based therapeutics may need to be tuned so that they have desirable characteristics (for example, desirable biodistribution, half-lives, etc.) after administration to a subject. Some antibody-based therapeutics have a format that differs from that of a native immunoglobulin molecule. For example, in some therapeutics, an antibody or antibody fragment is fused to another polypeptide; in some, the antibody or antibody fragment(s) is in a configuration or has a valence not found in nature. These antibody-based therapeutics may also need to be tuned.

Thus, there remains a need for optimized antibody-based therapeutics.

### SUMMARY

The present invention addresses this need with the provision of a suite of optimized self-assembled polypeptide complexes comprising antibody fragments. Depending on the desired characteristics (e.g., pharmacokinetic characteristics), a particular self-assembled polypeptide complex or set of complexes can be chosen for use. For example, in certain embodiments, provided are self-assembled polypeptide complexes that have half-lives or other pharmacokinetic characteristics similar to that of IgG molecules.

In accordance with an aspect, there is provided a self-assembled polypeptide complex comprising
(a) a plurality of first fusion polypeptides, each first fusion polypeptide comprising (1) an Fc polypeptide linked to (2) a nanocage monomer or subunit thereof, wherein the Fc polypeptide comprises a Fc chain having one or more mutations relative to a reference Fc chain of the same Ig class, and
(b) a plurality of second fusion polypeptides, each second fusion polypeptide comprising (1) an antigen-binding antibody fragment linked to (2) a nanocage monomer or subunit thereof.

In an aspect, (1) if the Fc polypeptide is an IgG1 Fc polypeptide, the antigen-binding fragment is not a Fab fragment that binds to SARS-CoV-2; and/or (2) if the nanocage monomer is a mouse ferritin monomer and the Fc polypeptide is a mouse IgG2a Fc polypeptide, the antigen-binding antibody fragment is not a Fab fragment that binds to CD19.

In an aspect, the nanocage monomer is a ferritin monomer.

In an aspect, the ferritin monomer is a ferritin light chain.

In an aspect, the self-assembled polypeptide complex does not comprise any ferritin heavy chains or subunits of ferritin heavy chains.

In an aspect, the ferritin monomer is a human ferritin.

In an aspect, the Fc polypeptide is an IgG1 Fc polypeptide.

In an aspect, the Fc polypeptide is an IgG2 Fc polypeptide.

In an aspect, the Fc polypeptide is a single-chain Fc (scFc).

In an aspect, the Fc polypeptide is an Fc monomer.

In an aspect, the antigen-binding antibody fragment comprises a light chain variable domain and a heavy chain variable domain.

In an aspect, the antigen-binding antibody fragment is a Fab fragment.

In an aspect, each second fusion polypeptide does not comprise any CH2 or CH3 domains.

In an aspect, the one or more mutations comprise a mutation or set of mutations associated with altered binding to FcRn.

In an aspect, the mutation or set of mutations comprises a mutation at one or more of the following residues: M252, I253, S254, T256, K288, M428, and N434, or combinations thereof, wherein numbering is according to the EU index.

In an aspect, the mutation or set of mutations comprises mutations at M428 and N434, wherein numbering is according to the EU index.

In an aspect, the mutation or set of mutations comprises M428L and N434S mutations, wherein numbering is according to the EU index.

In an aspect, the altered binding to FcRn is decreased binding to FcRn.

In an aspect, the mutation or set of mutations associated with decreased binding to FcRn is selected from the group consisting of I253A, I253V, and K288A, and combinations thereof, wherein numbering is according to the EU index.

In an aspect, the one or more mutations comprise a mutation or set of mutations associated with altered effector function.

In an aspect, the Fc polypeptide is an IgG1 Fc polypeptide, and wherein the mutation or set of mutations comprises a mutation at one or more the following residues: L234, L235, G236, G237, P329, and A330, or combinations thereof, wherein numbering is according to the EU index.

In an aspect, the altered effector function is decreased effector function.

In an aspect, the mutation or set of mutations associated with decreased effector function is selected from the group consisting of LALA (L234A/L235A), LALAP (L234A/L235A/P329G), G236R, G237A, and A330L, wherein numbering is according to the EU index.

In an aspect, the nanocage monomer or subunit thereof is a ferritin monomer subunit, and
a. each first fusion polypeptide comprises a ferritin monomer subunit which is C-half-ferritin and each second fusion polypeptide comprises a ferritin monomer subunit which is N-half-ferritin; or
b. each first fusion polypeptide comprises a ferritin monomer subunit which is N-half ferritin and each second fusion polypeptide comprises a ferritin monomer subunit which is C-half-ferritin.

In an aspect, the self-assembled polypeptide complex is characterized by a 1:1 ratio of first fusion polypeptides to second fusion polypeptides.

In an aspect, within each first fusion polypeptide, the Fc polypeptide is linked to the nanocage monomer or subunit thereof via an amino acid linker.

In an aspect, within each first fusion polypeptide, the Fc polypeptide is linked to the nanocage monomer or subunit thereof at the N-terminus of the nanocage monomer or subunit thereof.

In an aspect, within each second fusion polypeptide, the antigen-binding antibody fragment is linked to the nanocage monomer or subunit thereof via an amino acid linker.

In an aspect, within each second fusion polypeptide, the antigen-binding antibody fragment is linked to the nanocage monomer or subunit thereof at the N-terminus of the nanocage monomer or subunit thereof.

In an aspect, the self-assembled polypeptide complex further comprises a plurality of third fusion polypeptides, each third fusion polypeptide comprising (1) an antigen-binding antibody fragment linked to (2) a nanocage monomer or a subunit thereof, wherein the third fusion polypeptide is different than the second fusion polypeptide.

In an aspect, the antigen-binding antibody fragment within the third fusion polypeptide comprises a light chain variable domain and a heavy chain variable domain.

In an aspect, the antigen-binding antibody fragment within the third fusion polypeptide is a Fab fragment.

In an aspect, each third fusion polypeptide does not comprise any CH2 or CH3 domains.

In an aspect, the antigen-binding antibody fragment of the second fusion polypeptide is capable of binding a first epitope, the antigen-binding fragment of the third fusion polypeptide is capable of binding a second epitope, and the first epitopes and second epitopes are distinct and non-overlapping.

In an aspect, first epitopes and second epitopes are from the same protein.

In an aspect, the self-assembled polypeptide complex comprises a total of 24 to 48 fusion polypeptides.

In an aspect, the self-assembled polypeptide complex comprises a total of at least 24 fusion polypeptides.

In an aspect, the self-assembled polypeptide complex comprises a total of at least 32 fusion polypeptides.

In an aspect, the self-assembled polypeptide complex has a total of about 32 fusion polypeptides.

In an aspect, the half-life of the self-assembled polypeptide complex is at least 3 days, at least 5 days, at least 7 days, at least 10 days, at least 14 days, at least 21 days, or at least 28 days when administered to a subject in need thereof.

In an aspect, the self-assembled polypeptide complex is characterized in that, after administration of a composition comprising the self-assembled polypeptide complex, the self-assembled polypeptide complex has a half-life substantially similar to that of a reference IgG molecule administered by the same route of administration and in a similar composition.

In an aspect, the reference IgG molecule is an antibody from which the antigen-binding antibody fragment within the second fusion polypeptide is derived or is an antibody from which the antigen-binding antibody fragment within the third fusion polypeptide is derived.

In an aspect, the half-life of the self-assembled polypeptide complex is from about 3 to about 35 days when administered to a subject in need thereof.

In an aspect, the self-assembled polypeptide complex is detectable in serum after at least 3 days, at least 5 days, at least 7 days, at least 10 days, at least 14 days, at least 21 days, or at least 28 days after administration to a subject in need thereof.

In an aspect, the area-under-the-curve (AUC) of the self-assembled polypeptide complex is at least 10 day·μg/mL, at least 25 day·μg/mL, at least 50 day·μg/mL, at least 100 day·μg/mL, at least 200 day·μg/mL, at least 300 day·μg/mL, at least 400 day·μg/mL, at least 500 day·μg/mL, at least 750 day·μg/mL, at least 1000 day·μg/mL, at least 1500 day·μg/mL, at least 2000 day·μg/mL, at least 2500 day·μg/mL, at least 3000 day·μg/mL, at least 4000 day·μg/mL, at least 5000 day·μg/mL, at least 6000 day·μg/mL, at least 7000 day·μg/mL, or at least 8000 day·μg/mL when administered to a subject in need thereof.

In an aspect, the area-under-the-curve (AUC) of the self-assembled polypeptide complex is from about 10 to about 8000 day·μg/mL when administered to a subject in need thereof.

In an aspect, the maximum concentration (Cₘₐₓ) of the self-assembled polypeptide complex is at least 10 μg/mL, at least 25 μg/mL, at least 50 μg/mL, at least 100 μg/mL, at least 250 μg/mL, at least 500 μg/mL, at least 750 μg/mL, at least 1 mg/mL, at least 10 mg/mL, at least 25 mg/mL, at least 50 mg/mL, at least 75 mg/mL, at least 100 mg/mL, at least 250 mg/mL, at least 500 mg/mL, or at least 750 mg/mL when administered to a subject in need thereof.

In an aspect, the maximum concentration (Cₘₐₓ) of the self-assembled polypeptide complex is from about 10 μg/mL to about 750 mg/mL when administered to a subject in need thereof.

In an aspect, the subject is human.

In an aspect, administration to the subject is by parenteral administration.

In an aspect, administration to the subject is by subcutaneous administration, intravenous administration, intramuscular administration, intranasal administration, or by inhalation.

In an aspect, the self-assembled polypeptide complex is characterized in that the self-assembled polypeptide complex induces antibody-dependent cellular phagocytosis (ADCP) in an in vitro model of ADCP.

In an aspect, the ADCP is induced at a level of at least 20%, at least 25%, at least 30%, at least 35%, at least 40%, at least 45%, at least 50%, at least 55%, or at least 60% internalization of target.

In accordance with an aspect, there is provided a method comprising administering a composition comprising the self-assembled polypeptide complex described herein to a mammalian subject.

In an aspect, the subject is human.

In an aspect, the method comprises administration by a systemic route.

In an aspect, the systemic route comprises subcutaneous, intravenous, or intramuscular injection, inhalation, or intranasal administration.

In an aspect, after administration, the half-life of the self-assembled polypeptide complex in the mammalian subject is at least 3 days, at least 5 days, at least 7 days, at least 10 days, at least 14 days, at least 21 days, or at least 28 days.

In an aspect, after administration, the half-life of the self-assembled polypeptide complex in the mammalian subject is from 3 to 35 days.

In an aspect, after administration, the area-under-the-curve (AUC) of the self-assembled polypeptide complex in the mammalian subject is at least 10 day·μg/mL, at least 25 day·μg/mL, at least 50 day·μg/mL, at least 100 day·μg/mL, at least 200 day·μg/mL, at least 300 day·μg/mL, at least 400 day·μg/mL, at least 500 day·μg/mL, at least 750 day·μg/mL, at least 1000 day·μg/mL, at least 1500 day·μg/mL, at least 2000 day·μg/mL, at least 2500 day·μg/mL, at least 3000 day·μg/mL, at least 4000 day·μg/mL, at least 5000 day·μg/mL, at least 6000 day·μg/mL, at least 7000 day·μg/mL, or at least 8000 day·μg/mL.

In an aspect, after administration, the area-under-the-curve (AUC) of the self-assembled polypeptide complex in the mammalian subject is from about 10 to about 8000 day·μg/mL.

In an aspect, after administration, the maximum concentration (Cmax) of the self-assembled polypeptide complex in the mammalian subject is at least 10 μg/mL, at least 25 μg/mL, at least 50 μg/mL, at least 100 μg/mL, at least 250 μg/mL, at least 500 μg/mL, at least 750 μg/mL, at least 1 mg/mL, at least 10 mg/mL, at least 25 mg/mL, at least 50 mg/mL, at least 75 mg/mL, at least 100 mg/mL, at least 250 mg/mL, at least 500 mg/mL, or at least 750 mg/mL.

In an aspect, after administration, the maximum concentration (Cmax) of the self-assembled polypeptide complex in the mammalian subject is from about 10 μg/mL to about 750 mg/mL.

In accordance with an aspect, there is provided a fusion polypeptide comprising (1) an Fc polypeptide linked to (2) a nanocage monomer or subunit thereof, wherein the Fc polypeptide comprises a Fc chain having one or more mutations relative to a reference Fc chain of the same Ig class, wherein the one or more mutations comprise a mutation or set of mutations associated with altered binding to FcRn and/or with altered effector function.

In an aspect, the nanocage monomer is a ferritin monomer.

In an aspect, the ferritin monomer is a ferritin light chain.

In an aspect, the fusion polypeptide does not comprise any ferritin heavy chains or subunits of ferritin heavy chains.

In an aspect, the ferritin monomer is a human ferritin.

In an aspect, the Fc polypeptide is an IgG1 Fc polypeptide.

In an aspect, the Fc polypeptide is an IgG2 Fc polypeptide.

In an aspect, the Fc polypeptide is a single-chain Fc (scFc).

In an aspect, the mutation or set of mutations comprises a mutation at one or more of the following residues: M252, I253, S254, T256, K288, M428, and N434, or combinations thereof, wherein numbering is according to the EU index.

In an aspect, the altered binding to FcRn is decreased binding to FcRn.

In an aspect, the mutation or set of mutations associated with decreased binding to FcRn is selected from the group consisting of I253A, I253V, and K288A, and combinations thereof, wherein numbering is according to the EU index.

In an aspect, the Fc polypeptide is an IgG1 Fc polypeptide, and wherein the mutation or set of mutations comprises a mutation at one or more the following residues: L234, L235, G236, G237, P329, and A330, or combinations thereof, wherein numbering is according to the EU index.

In an aspect, the altered effector function is decreased effector function.

In an aspect, the mutation or set of mutations associated with decreased effector function is selected from the group consisting of LALA (L234A/L235A), LALAP (L234A/L235A/P329G), G236R, G237A, and A330L, wherein numbering is according to the EU index.

In an aspect, the nanocage monomer or subunit thereof is a ferritin monomer subunit, and
a. each first fusion polypeptide comprises a ferritin monomer subunit which is C-half-ferritin and each second fusion polypeptide comprises a ferritin monomer subunit which is N-half-ferritin; or
b. each first fusion polypeptide comprises a ferritin monomer subunit which is N-half ferritin and each second fusion polypeptide comprises a ferritin monomer subunit which is C-half-ferritin.

In an aspect, within each first fusion polypeptide, the Fc polypeptide is linked to the nanocage monomer or subunit thereof via an amino acid linker.

In an aspect, within each first fusion polypeptide, the Fc polypeptide is linked to the nanocage monomer or subunit thereof at the N-terminus of the nanocage monomer or subunit thereof.

### BRIEF DESCRIPTION OF THE DRAWINGS

FIG. 1A is a diagrammatic representation of human ferritin light chain (hFTL) and exemplary N-half ferritin and C-half ferritin molecules.

FIGS. 1B, 1C, and 1D are diagrammatic representations of exemplary Multabodies formation of the disclosure.

FIG. 2A are negative stain electron micrographs of T-01 MB, T-02 MB, and T-01 MB.v2, each containing wild-type IgG1 Fc.

FIG. 2B are negative stain electron micrographs of T-01 MB containing various Fc.

FIGS. 3A and 3B provide biolayer interferometry (BLI) concentration-response curves for the binding of T-01 MB containing various Fc to human FcRn at pH 5.6 and pH 7.4, respectively.

FIGS. 3C, 3D, and 3E provide BLI concentration-response curves for the binding of T-01 MB containing various Fc to human FcγRIIa, human FcγRIIb, and FcγRI, respectively.

FIG. 3F provides BLI concentration-response curves for the binding of T-01 MB.v2 containing wild-type IgG1 Fc or Fc IgG1 with M428L/N434S (LS) mutations to human Fc receptors.

FIG. 4A provides BLI concentration-response curves for the binding of T-01 MB, T-02 MB, and T-01 MB.v2 to target/epitope of PGDM1400, N49P7, 10E8v4, or iMab, as included as Fab in the Multabodies.

FIGS. 4B, 4C, and 4D provide BLI concentration-response curves for the binding of T-01 MB containing various Fc to the target/epitope of PGDM1400 (BG5050 SOSIP.664 D368R), N49P7 (93TH057), and 10E8v4 (gp41 MPER), respectively.

FIG. 4E provides BLI concentration-response curves for the binding of PGDM1400, N49P7, 10E8v4, or iMab IgG to BG5050 SOSIP.664 D368R, 93TH057, gp41 MPER, and CD4.

FIG. 5A illustrates an experiment described in Example 4 and conducted using CB17/Icr-Prkdc^{scid}/IcrIcoCrl immunodeficient (SCID) mice. FIGS. 5B, 5C, 5D, and 5E illustrates serum levels of testing Multabodies or IgG1 control following subcutaneous administration in SCID mice.

FIG. 6A illustrates an experiment described in Example 4 and conducted using NOD/Shi-scid/IL-2Rγnull immunodeficient (NCG) mice. FIG. 6B illustrates serum levels of testing Multabodies or IgG1 control following subcutaneous administration in NCG mice.

FIG. 6C provides body weights of NCG mice upon administration of testing Multabodies or IgG1 control. Mean values±SD for n=3 mice are displayed.

FIG. 7 illustrates the dose-dependent phagocytosis induced by testing Multabodies or controls, quantified and represented as the percentage increase in the internalization of 93TH057-coated microspheres compared to non-coated microspheres. *P<0.05, ***P<0.001 and P ****<0.0001. n=four biologically-independent samples.

FIGS. 8A, 8B, and 8C illustrate the breadth and median IC₅₀ values (μg/mL) of testing Multabodies (T-01 MB, T-01 MB.v2, and T-02 MB., respectively) (diamond), parental antibodies PGDM1400, N49P7, and 10E8v4 (circles), IgG1 control (triangle), and N6/PGDM1400x10E8v4 trispecific antibody (inverted triangle), determined with a TZM-bl assay described in Example 6.

FIG. 8D illustrates the dose-dependent neutralization of HIV-1 by T-01 MB containing various Fc, determined with a TZM-bl assay described in Example 6.

FIG. 9A illustrates the inhibition of CXCR4-tropic HIV-1 isolate IIIB infection of PBMCs by T-01 MB, T-01 MB.v2, or IgG1 control. The mean values±SD for three technical replicates are shown. FIG. 9B shows the percent of viable cells following T-01 MB, T-01 MB.v2, or IgG1 control relative to untreated control cells.

FIGS. 10A and 10B show 4-week stability under temperature stress conditions (40° C.) of T-01 MB and T-01 MB.v2. See Example 8.

FIG. 11. HIV-1 bNAb multimerization increases neutralization potency. Schematic of the self-assembly of (A) apoferritin (24 subunits) and (B) single-chain Fab-apoferritin fusions. Fab light chain (LC) and heavy chain (HC) are shown in light and dark pink, respectively, and are connected to the N terminus of the light chain of human apoferritin (grey) through a GGS-like flexible linker (dark). (C) Schematic representation of different Fab densities displayed on human apoferritin. Co-transfection of scFab-human apoferritin-encoding plasmids together with unconjugated apoferritin at ratios of 1:4 (dark yellow), 1:1 (black), 4:1 (blue) and 1:0 (red) resulted in molecules with different scFab valency, as confirmed by elution volumes in size exclusion chromatography and less unconjugated apoferritin in SDS-PAGE. Negative stain electron micrographs of the samples with the lowest and highest scFab valency are shown (scale bar 50 nm). (D) Avidity effect on neutralization of five bNAbs against a five-PsV panel (PVO.04, JRCSF, BG505 T332N, THR04156.18 and t278-50). Fold potency increase was calculated as the parental IgG IC₅₀ (μg/mL) divided by the Fab-apoferritin fusion IC₅₀ (μg/mL). Fold potency increase analyses were omitted in the following cases: N49P7-t278-50, VRC01-T278-50 and 10-1074-THRO4156.18 due to neutralization resistance. Bars (±SD) represent the mean value from n=3 biologically independent samples.

FIG. 12. Characterization of scFab-apoferritin fusions. (A) SDS-PAGE bands corresponding to scFab-apoferritin and unconjugated apoferritin were quantified by densitometry using the ImageJ software (rsb.info.nih.gov/ij/). Intensity plots of the bands in each lane (yellow box) are shown (B). (C) The approximate number of scFabs displayed on the particles was calculated as follows: intensity of scFab band/total intensity, and compared with the theoretical numbers inferred from DNA ratios used for co-transfection.

FIG. 13. Design, assembly and neutralization profile of HIV Multabodies against a 14-PsV panel. (A) Schematic of the human apoferritin split design that drives hetero-dimerization of scFab-human apoferritin subunits. (B) Size exclusion chromatography in-line with multi-angle light scattering of 24-mer PGDM1400 scFab-apoferritin particles (black) and T-01 MB (dark red). The molar mass of each elution peak (lines under UV absorbance) is shown in MDa. (C) Negative stain electron micrographs of T-01 MB (scale bar 50 nm). (D) Concentration-response curves for binding of T-01 MB to multiple epitopes. PGDM1400, N49P7 and 10E8 binding sites are colored in red, blue and pink, respectively on the surface representation of the HIV-1 Env trimer (grey). Red lines represent raw data; black lines represent global fits. (E) Breadth (cutoff IC₅₀ set at 10 μg/mL) and median IC₅₀ values (μg/mL) of T-01 MB (red diamond), parental bNAbs (white circles), IgG combination (gray triangle) and the N6/PGDM1400x10 E8v4 tri-specific antibody (black triangle). The 14-PsV panel was selected based on susceptibility and resistance to the parental IgGs. (F) Individual IC₅₀ values (μg/mL) to each PsV variant. The solid line denotes the median neutralization IC₅₀ of all 14 viral strains. Those pseudoviruses that show the highest neutralization resistance are highlighted by a red box. IC₅₀ values in (D) and (E) were calculated from three biological replicates.

FIG. 14. Multabody affinity-purification scheme. Protein A and Protein L sequential affinity purification. Binding to Protein A enriches for Multabodies with Fc (green), while Protein L enriches for Multabodies with the kappa chain Fab PGDM1400 (blue). Complementation of the two halves of the apoferritin split design ensures the presence of N49P7/iMab (orange) and 10E8v4 scFabs (pink) (fused to C-ferritin) during the protein A purification step. An alanine-to-proline point mutation at position 12 of the kappa chain of iMab was introduced to disrupt binding to Protein L. Gel filtration is performed to separate any aggregated material or non-assembled component.

FIG. 15. Generation of a Multabody that cross-targets the HIV-1 Env and the CD4 receptor. (A) Schematic representation of MB components. (B) Size exclusion chromatography in-line with multi-angle light scattering of 24-mer PGDM1400 scFab-apoferritin particles (black) and T-02 MB (blue). The molar mass of each sample is shown in MDa. (C) Negative stain electron micrographs (scale bar 50 nm) and (D) binding profile of T-02 MB. (E) Breadth (cutoff IC₅₀ set at 10 μg/mL) and median IC₅₀ values (μg/mL) of T-02 MB (red diamond) in comparison to its parental bNAbs (white circles) and IgG combination (gray triangle). The mean IC₅₀ values (μg/mL) derived from three biological replicates for those pseudoviruses that show the highest neutralization resistance to T-02 MB are shown.

FIG. 16. Biophysical characterization of HIV-1 Multabodies. Comparison of the Tₘ and T_{agg} temperatures of T-01/T-02 MB, 12-mer ferritin fusions, parental IgGs and the N6/PGDM1400x10 E8v4 tri-specific antibody.

FIG. 17. Binding characteristics of IgGs binding to four different antigens. BLI response curves of IgG binding to 93TH057 gp120, BG505 SOSIP.664_D368R, MPER peptide and CD4 immobilized onto Ni-NTA biosensors. The BG505 SOSIP.664_D368R trimer and 93TH057 gp120 monomer were selected as epitope-specific ligands for PGDM1400 and N49P7, respectively. Red lines represent raw data; black lines represent global fits.

FIG. 18. Engineering and biophysical characterization of Multabody v2. (A) The second-generation Multabody design displays two distinct features in comparison to the original Multabody design: 1) the Fc (green) is fused to the C terminus of the second half of apoferritin in the split ferritin design; and 2) the single-chain Fc domain (green), fused to the C terminus of an apoferritin half protomer is reverted to a monomeric Fc chain. Dimerization of each Fc in MB.v2 drives assembly of four Fabs (two Fab2 and two Fab3—bottom row) while only one Fab is assembled per Fc into the previous MB version (top row). (B) Negative stain electron micrographs of T-01 MB.v2 (scale bar 50 nm). (C) Concentration-response curves for binding of T-01 MB.v2 to multiple epitopes. Red lines represent raw data; black lines represent global fits. (D) Comparison of T_{agg}, and (E) long-term stability under temperature stress conditions (10 mg/ml; 40° C.) of the two different Multabody versions. PsV neutralization (mean values±SD for two technical replicates) comparison at week 0 vs week 4 is shown.

FIG. 19. Multabody v2 features. (A) Modification of the fusion of the Fc (green) from the N terminus of N-ferritin (top row) to the C terminus of C-ferritin (bottom row) inverts the orientation of the Fc in the Multabody. (B) Fc dimerization of two Fc chains fused to the C terminus of two independent ferritin subunits at the 4-fold symmetry axes of the apoferritin nanocage acts as an additional driver of Multabody v2 assembly.

FIG. 20. Fine-tuning of Fc on Multabody for IgG-like characteristics. (A) Concentration-response curves for pH dependent binding to human FcRn by T-01 MB and T-01 MB.v2. (B) Comparison of the FcRn apparent binding affinities (K_{D}) at acidic pH between MBs and an IgG1. n=3 biologically independent samples are shown. Apparent K_{D} lower than 10⁻¹² M (dash black line) is beyond the instrument detection limit. (C) Concentration-response curves to the high-affinity human FcγRI (top), and low-affinity human FcγRIIa (bottom). (D) Dose-dependent phagocytosis determined as a percentage increase in internalization of 93TH057-coated fluorescent microspheres compared to a no-antibody control. Anti-human FcR binding inhibitor antibody was added to block Fc-mediated internalization (dark red). Data were analyzed by two-way ANOVA with Tukey's multiple comparisons test. Each group was compared to the IgG negative control. *P<0.05, ***P<0.001 and P ****<0.0001. IgG and MB samples with no affinity for the antigen coated beads were added as control samples. n=four biologically-independent samples. (E) Serum levels after subcutaneous administration of 5 mg/kg of Multabodies or parental IgG mixture in female NOD/Shi-scid/IL-2Rγnull (NCG) immunodeficient mice. (F) Body weight upon administration of 5 mg/kg of molecules in NCG mice. Mean values±SD for n=3 mice are shown in (E) and (F).

FIG. 21. Broad and potent neutralization by Multabody v2 against extended HIV-1 PsV panels. (A) Breadth and median IC₅₀ values (μg/mL) of T-01 Multabody versions (different shades of red diamond), individual IgGs (black circles) and IgG mixture (blue triangle) against a 25-PsV panel with 56% of PsV variants resistant to PGDM1400 neutralization. (B) Individual IC₅₀ values (μg/mL) for each PsV variant. IC₅₀ values in (A) and (B) were calculated from three biological replicates. (C) Potency (IC₅₀)-breadth (left) and potency (IC₈₀)-breadth (right) curve comparison of T-01 Multabody versions, as well as parental IgGs and an IgG mixture against an extended multiclade panel of 118 HIV-1 PsV variants. (D) Individual IC₅₀ (left) and IC₈₀ (right) values for each PsV variant in (C). Yellow dots correspond to IC₅₀ values from PsVs that are highly resistant to PGDM1400 neutralization. The solid line in (B) and (D) denotes the median IC₅₀ neutralization titer of all viral strains in each panel.

FIG. 22. PsV neutralization and inhibition of primary PBMC infection by Multabodies. (A) Molar potency (IC₅₀)-breadth (left) and molar potency (IC₈₀)-breadth (right) graphs comparing T-01 Multabody versions, as well as parental IgGs and an IgG mixture against a panel of 118 HIV-1 PsV variants. (B) Measurement of HIV-1 replication via p24 detection in PBMC culture supernatant derived from three different blood donors. Data shown represent HIV-1 replication levels at 7 days post-infection with the CXCR4-tropic HIV-1 isolate IIIB. Mean values±SD for three technical replicates are shown. (C) Impact of IgG mix and Multabody treatments on cell viability, showing the percent of viable cells relative to untreated control cells under the same experimental conditions as in (B).

### DETAILED DESCRIPTION

The inventors have previously described self-assembled polypeptide complexes comprising fusion polypeptides that comprise antibody fragments. These self-assembled polypeptide complexes can be designed and adapted for a variety of therapeutic purposes. For example, as described herein, a self-assembled polypeptide complex comprising both Fab- and Fc-containing fusion polypeptides may be used to target cells expressing an antigen to which the Fab can bind, and the Fc portion may mediate interactions with other molecules in the body.

In many situations, it may be desirable to optimize the behavior of these self-assembled polypeptide complexes after administration, e.g., by modulating characteristics such as half-life and/or ability to mediate antibody-mediated effects. Techniques for optimizing IgG molecules are known in the art. For example, both half-life and the certain antibody-mediated effects can be modulated using modifications to the Fc region of an IgG molecule.

However, the inventors have surprisingly discovered that, in some situations, the techniques for optimizing IgG molecules, when applied to the inventors' self-assembled polypeptide complexes, may have unexpected effects. As one example, the inventors have discovered that an Fc modification typically associated with reduced FcRn binding (and reduced half-life) in the context of an IgG1 molecule actually conferred more desirable bioavailability characteristics in the context of the inventors' self-assembled polypeptide complexes.

Utilizing these and other insights, the inventors have developed a suite of a self-assembled polypeptide complexes, each of which is optimized for a particular desired outcome, and related methods.

For example, in certain embodiments, after administration to a subject, a provided self-assembled polypeptide complex has one or more pharmacokinetic features similar to that of a reference IgG molecule (e.g., an IgG molecule whose class matches the class of an Fc chain within an Fc polypeptide within the self-assembled polypeptide complex). For example, in some embodiments, a self-assembled polypeptide complex as disclosed herein has a similar bioavailability to that of reference IgG molecule. In some embodiments, a self-assembled polypeptide complex as disclosed herein has a similar half-life to that of reference IgG molecule.

In certain embodiments, after administration to a subject, a provided self-assembled polypeptide complex induces antibody-dependent cellular phagocytosis (ADCP).

### Definitions

The terms “about” and “approximately,” when used herein in reference to a value, are used interchangeably and refer to a value that is similar to the referenced value. In general, those skilled in the art, familiar with the context, will appreciate the relevant degree of variation encompassed by “about” or “approximately” in that context. For example, in some embodiments, the terms “about” and “approximately” may encompass a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the terms “alter,” “altered,” “decrease,” “decreased,” “increase,” “increased,” or “reduction,” “reduced,” (e.g., in reference to certain outcomes or effects) have meanings relative to a reference level. In some embodiments, in the context of discussing mutations in an Fc chain or Fc polypeptide, the reference level is a level known or as determined with an IgG that does not contain the referenced mutation(s) in the Fc region.

The terms “ferritin” and “apoferritin” are used interchangeably herein and generally refer to a polypeptide (e.g., a ferritin chain) that is capable of assembling into a ferritin complex which typically comprises 24 protein subunits. In some embodiments, the ferritin is a human ferritin, e.g., a human ferritin light chain, e.g., a human ferritin light chain having at least 85% sequence identity to SEQ ID NO:1 or UniProt P02792. In some embodiments, the ferritin is a wild-type ferritin. For example, the ferritin may be a wild-type human ferritin.

The term “ferritin monomer,” is used herein to refer to a single chain of a ferritin that, in the presence of other ferritin chains, is capable of self-assembling into a polypeptide complex comprising a plurality of ferritin chains, e.g., 24 or more ferritin chains.

As used herein, the term “linker” is used to refer to an entity that connects two or more elements to form a multi-element agent. For example, those of ordinary skill in the art appreciate that a polypeptide (e.g., fusion polypeptide) whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker (L). In some embodiments, the linker is an “amino acid linker,” that is, it comprises amino acid residues, e.g., an amino acid linker may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acid residues. In some embodiments, a linker is characterized in that it tends not to adopt a rigid three-dimensional structure, but rather provides flexibility to the polypeptide.

The term “multispecific,” as used herein, refers to the characteristic of having at least two binding sites at which at least two different binding partners, e.g., an antigen or receptor (e.g., Fc receptor), can bind. For example, a polypeptide complex that comprises at least two Fab fragments, wherein each of the two Fab fragments is capable of binding to a different antigen, is “multispecific.” As an additional example, a polypeptide complex that comprises an Fc fragment (which is capable of binding to an Fc receptor) and a Fab fragment (which is capable of binding to an antigen) is “multispecific.”

The term “multivalent,” as used herein, refers to the characteristic of having at least two binding sites at which a binding partner, e.g., an antigen or receptor (e.g., Fc receptor), can bind. The binding partners that can bind to the at least two binding sites may be the same or different.

The term “nanocage monomer,” as used herein, refers to a single chain of a polypeptide that is capable of self-assembling with other nanocage monomers to form a self-assembled polypeptide complex comprising a plurality of nanocage monomers. In some embodiments, the nanocage monomer is selected from monomers of ferritin, apoferritin, encapsulin, sulfur oxygenase reductase (SOR), lumazine synthase, pyruvate dehydrogenase, carboxysome, vault proteins, GroEL, heat shock protein, E2P coat protein, MS2 coat protein, fragments thereof, and variants thereof.

The term “polypeptide,” as used herein, generally has its art-recognized meaning of a polymer of at least three amino acids, e.g., linked to each other by peptide bonds. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

The term “self-assembled,” when used in reference to a macromolecular complex (e.g., a polypeptide complex), refers to the spontaneous formation of that complex when sufficient constituents of the complex (e.g., fusion polypeptides) to be formed are present. In some embodiments, complexes self-assemble in physiological conditions, or in a buffer (e.g., a solution) that corresponds to physiological conditions.

As used herein, the term “subject” to an organism, typically a mammal (e.g., a human). In some embodiments, a subject is suffering from or susceptible to a relevant disease, disorder or condition. In some embodiments, a subject displays one or more symptoms or characteristics of a disease, disorder or condition. In some embodiments, a subject is someone with one or more features characteristic of susceptibility to or risk of a disease, disorder, or condition. In some embodiments, a subject is a patient. In some embodiments, a subject is a subject to whom diagnosis and/or therapy is and/or has been administered.

As used herein, the term “treatment” (also “treat” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### A. Fusion Polypeptides

In many embodiments, fusion polypeptides compatible with compositions and methods disclosed herein generally comprise a nanocage monomer or subunit thereof linked to either an Fc polypeptide or to an antigen-binding antibody fragment. Within the fusion polypeptide, the Fc polypeptide or the antigen-binding antibody fragment may be linked to the nanocage monomer or subunit thereof at a particular terminus of the nanocage monomer or subunit thereof, e.g., the N-terminus or the C-terminus. In some embodiments, the Fc polypeptide or antigen-binding antibody fragment is linked via an amino acid linker, such as a linker as described herein.

In some embodiments, (1) if the Fc polypeptide is an IgG1 Fc polypeptide, the antigen-binding fragment is not an Fab fragment that binds to SARS-CoV-2; and/or (2) if the nanocage monomer is a mouse ferritin monomer and the Fc polypeptide is a mouse IgG2a Fc polypeptide, the antigen-binding antibody fragment is not an Fab fragment that binds to CD19.

### 1. Nanocage Monomers and Subunits Thereof

In some embodiments, the nanocage monomer is a ferritin monomer.

The term “ferritin monomer,” is used herein to refer to a single chain of a ferritin that, in the presence of other ferritin chains, is capable of self-assembling into a polypeptide complex comprising a plurality of ferritin chains, e.g., 24 or more ferritin chains. In some embodiments, the ferritin monomer is a ferritin light chain. In some embodiments, the ferritin monomer does not include a ferritin heavy chain or other ferritin components capable of binding to iron.

In some embodiments, each fusion polypeptide within the self-assembled polypeptide complex comprises a ferritin light chain or a subunit of a ferritin light chain. In these embodiments, the self-assembled polypeptide complex does not comprise any ferritin heavy chains or subunits of ferritin heavy chains.

In some embodiments, the ferritin monomer is a human ferritin chain, e.g., a human ferritin light chain, e.g., a human ferritin light chain having the sequence of at least residues 2-175 of SEQ ID NO: 1. In some embodiments, the ferritin monomer is a mouse ferritin chain.

A “subunit” of a ferritin monomer refers to a portion of a ferritin monomer that is capable of spontaneously associating with another, distinct subunit of a ferritin monomer, so that the subunits together form a ferritin monomer, which ferritin monomer, in turn, is capable of self-assembling with other ferritin monomers to form a polypeptide complex.

In some embodiments, the ferritin monomer subunit comprises approximately half of a ferritin monomer. As used herein, the term “N-half ferritin” refers to approximately half of a ferritin chain, which half comprises the N-terminus of the ferritin chain. As used herein, the term “C-half ferritin” refers to approximately half a ferritin chain, which half comprises the C-terminus of the ferritin chain. The exact point at which a ferritin chain may be divided to form the N-half ferritin and the C-half ferritin may vary depending on the embodiment. In the context of ferritin monomer subunits based on human ferritin light chain, for example, the halves may be divided at a point that corresponds to a position from about position 75 to about position 100 of SEQ ID NO:1 (or a substantial portion thereof). For example, in some embodiments, an N-half ferritin based on a human ferritin light chain has an amino acid sequence corresponding to residues 1-95 of SEQ ID NO: 1 (or a substantial portion thereof), and a C-half ferritin based on a human ferritin light chain has an amino acid sequence corresponding to residues 96-175 of SEQ ID NO: 1 (or a substantial portion thereof).

In some embodiments, the halves are divided at a point that corresponds to a position from about position 85 to about position 92 of SEQ ID NO:1. For example, in some embodiments, an N-half ferritin based on a human ferritin light chain has an amino acid sequence corresponding to residues 1-90 of SEQ ID NO:1, and a C-half ferritin based on a human ferritin light chain has an amino acid sequence corresponding to residues 91-175 of SEQ ID NO:1.

### 2. Fc Polypeptides

In certain embodiments, fragment crystallizable (Fc) polypeptides comprise Fc chains that each have one or more mutations relative to a reference Fc chain of the same Ig class. As explained further herein below, the reference Fc chain may be of, e.g., the IgG1 or IgG2 class.

Unless otherwise noted, numbering of mutations within an antibody fragment, e.g., an Fc polypeptide, throughout this disclosure is according to the EU index.

In some embodiments, the Fc polypeptide is a human IgG Fc polypeptide, that is, except for mutations noted herein, the Fc polypeptide comprises an Fc chain that is substantially similar to that of an Fc chain within a wild type human IgG.

In some embodiments, the Fc polypeptide is an IgG1 Fc polypeptide (e.g., a human IgG1 Fc polypeptide), that is, except for mutations noted herein, the Fc polypeptide comprises an Fc chain that has an amino acid sequence that is substantially similar to that of the chains within a wild type IgG1 Fc. In some embodiments, the wild type IgG1 Fc is a human IgG1 Fc, in which each Fc chain has an amino acid sequence of SEQ ID NO:5.

For example, an IgG1 Fc polypeptide may comprise an Fc chain with an amino acid sequence that is at least 85%, at least 87.5%, at least 90%, at least 91%, at least 92%, at least 93%, at least 94%, at least 95%, at least 96%, at least 97%, at least 98%, or at least 99% identical to that of an Fc chain within a wild-type IgG1 Fc. In some embodiments, an IgG1 Fc polypeptide comprises an Fc chain that comprises the Fc mutations specifically described for that IgG1 Fc polypeptide, but has an amino acid sequence that is otherwise 100% identical to an Fc chain within a wild type IgG1 Fc. In some embodiments, the Fc polypeptide comprises an Fc chain that has an amino acid sequence that differs by at least one, at least two, at least three, or at least four amino acid residues from the sequence of SEQ ID NO:5. In some embodiments, the Fc polypeptide comprises an Fc chain that has an amino acid sequence that differs by no more than ten, no more than nine, no more than eight, no more than seven, no more than six, no more than five, or no more than four amino acid residues from the sequence of SEQ ID NO:5.

In some embodiments, the Fc polypeptide is an IgG2 Fc polypeptide, (e.g., a human IgG2 Fc polypeptide), that is, except for mutations noted herein, the Fc polypeptide comprises an Fc chain that has an amino acid sequence that is substantially similar to that of the chains within a wild type IgG2 Fc. In some embodiments, the wild type IgG2 Fc is a human IgG2 Fc, in which each Fc chain has an amino acid sequence of SEQ ID NO:46.

For example, an IgG2 Fc polypeptide may comprise an Fc chain with an amino acid sequence that is at least 85%, at least 87.5%, at least 90%, at least 91%, at least 92%, at least 93%, at least 94%, at least 95%, at least 96%, at least 97%, at least 98%, or at least 99% identical to that of an Fc chain within a wild-type IgG2a Fc. In some embodiments, an IgG2 Fc polypeptide comprises an Fc chain that comprises the Fc mutations specifically described for that IgG2 Fc polypeptide, but has an amino acid sequence that is otherwise 100% identical to an Fc chain within a wild type IgG2 Fc. In some embodiments, the Fc polypeptide comprises an Fc chain that has an amino acid sequence that differs by at least one, at least two, at least three, or at least four amino acid residues from the sequence of SEQ ID NO:46. In some embodiments, the Fc polypeptide comprises an Fc chain that has an amino acid sequences that differs by no more than ten, no more than nine, no more than eight, no more than seven, no more than six, no more than five, or no more than four amino acid residues from the sequence of SEQ ID NO:46.

In some embodiments, the Fc polypeptide is a single chain Fc (scFc), which comprises two Fc chains linked together by a covalent linker, e.g., via an amino acid linker.

In some embodiments, the Fc polypeptide is an Fc monomer, e.g., a single Fc chain having only one CH2 domain (second constant Ig domain of the heavy chain) and only one CH3 domain (third constant Ig domain of the heavy chain), which single Fc chain is typically capable of dimerizing with another single Fc chain.

In some embodiments, the one or more mutations comprises a mutation or set of mutations associated with an altered characteristic as further described herein. By “associated with,” it is meant that the mutation or set of mutations has been previously characterized, in the context of antibodies such as IgG antibodies, as conferring the altered characteristic (e.g., altered binding to FcRn, altered effector function, etc.) By “altered” it is meant that the characteristic (e.g., binding to an Fc receptor (e.g., FcRn)), is different than that observed without the mutation or set of mutations.

For example, in some embodiments, the altered characteristic comprises altered binding to an Fc receptor.

In some embodiments, the altered characteristic comprising altered binding to FcRn.

For example, the mutation or set of mutations associated with altered binding to FcRn may comprise a mutation at one or more residues selected from: M252, I253, S254, T256, K288, M428, N434, or combinations thereof.

In some embodiments, the altered binding to an Fc receptor comprises decreased binding to FcRn (e.g., decreased binding relative to a reference level corresponding to that level observed without the one or more mutations). For example, in some embodiments, the one or more mutations comprise a mutation or set of mutations associated with decreased binding to FcRn, e.g., I253A, I253V, K288A, or combinations thereof.

In some embodiments, the one or more mutations comprises a mutation or set of mutations associated with altered effector function, e.g., altered binding to an Fc receptor associated with effector function (e.g., Fcγ receptors such as FcγRI, FcγRII, or FcγRIIb).

For example, the one or more mutations may comprise a mutation or set of mutations at one or more residues selected from: L234, L235, G236, G237, P329, A330, and combinations thereof.

In some embodiments, the altered binding to an Fc receptor comprises decreased effector function, e.g., LALA (L234A/L235A), LALAP (L234A/L235A/P329G), G236R, G237A, A330L, or combinations thereof.

### 3. Antigen-Binding Antibody Fragments

In certain embodiments, the antigen-binding antibody fragment comprises a heavy chain variable region (e.g., a V_{H}). In certain embodiments, the antigen-binding antibody fragment comprises a heavy chain variable domain (e.g., V_{H}) and a light chain variable domain (e.g., a V_{L} or V_{K}). In certain embodiments, the antigen-binding antibody fragment comprises a Fab which comprises a heavy chain variable domain (e.g., V_{H}) and a light chain variable domain (e.g., a V_{L} or V_{K}).

In certain embodiments, the antigen-binding antibody fragment does not comprise any domains from the Fc region, e.g., does not comprise any CH2 or CH3 domains.

In some embodiments, the antigen-binding fragment binds to an antigen on an infectious disease agent, e.g., a virus.

In some embodiments, the antigen-binding antibody fragment binds to an antigen on a target cell, e.g., a cancer cell or an immune cell.

In embodiments where multiple types of fusion polypeptides having antigen-binding antibody fragments are used, the antigen-binding antibody fragments in the various types of fusion polypeptides may be capable of binding to the same epitope, or they may be capable of binding to epitopes that are distinct and non-overlapping. In some embodiments where the epitopes are distinct and non-overlapping, the epitopes are from the same protein.

### 4. Linkers

In certain embodiments, linkers are used within fusion polypeptides and/or within single-chain molecules such as scFcs. In some embodiments, the linker is an amino acid linker. For example, a linker as employed herein may comprise from about 1 to about 100 amino acid residues, e.g., about 1 to about 70, about 2 to about 70, about 1 to about 30, or about 2 to about 30 amino acid residues. In some embodiments, the linker comprises at least 2, at least 3, at least 4, at least 5, at least 6, at least 7, at least 8, at least 9, or at least 10 amino acid residues.

In certain embodiments, the linker comprises a glycine-serine sequence, e.g., a (GₙS)ₘ sequence (e.g., GGS, GGGS (SEQ ID NO:48), or GGGGS (SEQ ID NO:49) sequence) that is present in at least 1, at least 2, at least 3, at least 4, at least 5, at least 6, at least 7, at least 8, at least 9, at least 10, at least 11, at least 12, at least 13, or at least 14 copies within the linker.

### B. Self-Assembled Polypeptide Complexes

In one aspect, provided are self-assembled polypeptide complexes comprising a plurality of fusion polypeptides as disclosed herein. Generally, provided self-assembled polypeptide complexes comprise (a) a plurality of first fusion polypeptides, each first fusion polypeptide comprising (1) an Fc polypeptide linked to (2) a nanocage monomer or subunit thereof, wherein the Fc polypeptide comprises an Fc chain having one or more mutations relative to a reference Fc chain of the same Ig class, and (b) a plurality of second fusion polypeptides, each second fusion polypeptide comprising (1) an antigen-binding antibody fragment linked to (2) a nanocage monomer or subunit thereof.

In some embodiments, the nanocage monomer is a ferritin monomer, and each fusion polypeptide within the self-assembled polypeptide complex comprises a ferritin light chain or a subunit of a ferritin light chain. In these embodiments, the self-assembled polypeptide complex does not comprise any ferritin heavy chains, subunits of ferritin heavy chains, or other ferritin components capable of binding to iron.

In some embodiments, the nanocage monomer or subunit thereof is a ferritin monomer subunit, and (a) each first fusion polypeptide comprises a ferritin monomer subunit which is C-half-ferritin and each second fusion polypeptide comprises a ferritin monomer subunit which is N-half-ferritin; or (b) each first fusion polypeptide comprises a ferritin monomer subunit which is N-half ferritin and each second fusion polypeptide comprises a ferritin monomer subunit which is C-half-ferritin.

In some embodiments, the self-assembled polypeptide complex comprises from 24 to 48 fusion polypeptides in total. In some embodiments, the self-assembled polypeptide complex comprises 24 fusion polypeptides in total. In some embodiments, the self-assembled polypeptide complex comprises more than 24 fusion polypeptides, e.g., at least 26, at least 28, at least 30, at least 32 fusion polypeptides, at least 34 fusion polypeptides, at least 36 fusion polypeptides, at least 38 fusion polypeptides, at least 40 fusion polypeptides, at least 42 fusion polypeptides, at least 44 fusion polypeptides, at least 46 fusion polypeptides, or at least 48 fusion polypeptides in total. In some embodiments, the self-assembled polypeptide complex comprises about 32 fusion polypeptides.

In some embodiments, the self-assembled polypeptide complex comprises at least 4, at least 5, least 6, at least 7, or at least 8 first fusion polypeptides.

In some embodiments, the self-assembled polypeptide complex comprises at least 4, at least 5, least 6, at least 7, or at least 8 second fusion polypeptides.

In some embodiments, the self-assembled polypeptide complex further comprises at least 4, at least 5, least 6, at least 7, at least 8, at least 9, at least 10, least 11, at least 12, at least 13, at least 14, at least 15, or at least 16 third fusion polypeptides.

In some embodiments, the self-assembled polypeptide complex comprises a ratio of approximately 1:1, 1:2, 1:3, or 1:4 of first fusion polypeptides to all other fusion polypeptides.

### Pharmacokinetic Characteristics

In certain embodiments, when administered to a subject in need thereof, a provided self-assembled polypeptide complex, has one or more pharmacokinetic features similar to that of a reference IgG molecule (e.g., an IgG molecule whose class matches the class of an Fc chain within an Fc polypeptide of a first fusion polypeptide within the self-assembled polypeptide complex). In some embodiments, the ranges for the pharmacokinetic characteristics discussed herein (e.g., half-life, AUC, and/or Cₘₐₓ) are obtained when the self-assembled polypeptide complex is administered to a human subject. In some embodiments, the ranges for the pharmacokinetic characteristics discussed herein are obtained when the self-assembled polypeptide complex is administered via a systemic route, e.g., via intravenous or subcutaneous administration.

In some embodiments, a self-assembled polypeptide complex as disclosed herein has a similar half-life to that of reference IgG molecule. The reference IgG molecule may be, e.g., an antibody from which the antigen-binding antibody fragment within the second and/or third fusion polypeptide within the self-assembled polypeptide complex is derived. For example, if the antigen-binding fragment within the second and/or third fusion polypeptide comprises variable regions from “Antibody A,” then the reference IgG molecule may, in some embodiments, be “Antibody A.”

In some embodiments, after administration to a subject in need thereof, the self-assembled polypeptide complex has a half-life of from about 3 to 35 days, about 3 to about 28 days, about 3 to about 21 days, about 3 to about 14 days, about 3 to about 10 days, about 3 to about 7 days, about 3 to about 5 days, about 5 to about 35 days, about 5 to about 28 days, about 5 to about 21 days, about 5 to about 14 days, about 5 to about 10 days, about 5 to about 7 days, about 7 to about 35 days, about 7 to about 28 days, about 7 to about 21 days, about 7 to about 14 days, about 7 to about 10 days, about 10 to about 35 days, about 10 to about 28 days, about 10 to about 21 days, about 10 to about 14 days, about 14 to about 35 days, about 14 to about 28 days, about 14 to about 21 days, about 21 to about 35 days, or about 21 to about 28 days. In some embodiments, after administration to a subject in need thereof, the self-assembled polypeptide complex has a half-life of at least 3 days, at least 5 days, at least 7 days, at least 10 days, at least 14 days, at least 21 days, or at least 28 days. In some embodiments, after administration to a subject in need thereof, the self-assembled polypeptide complex is detectable in serum after at least 3 days, at least 5 days, at least 7 days, at least 10 days, at least 14 days, at least 21 days, or at least 28 days.

In some embodiments, a self-assembled polypeptide complex as disclosed herein has a similar bioavailability to that of reference IgG molecule, e.g., an antibody from which the Fab fragment comprised in the self-assembled polypeptide complex is derived. For example, in some embodiments, after administration to a subject in need thereof, the self-assembled polypeptide complex has an area-under-the-curve (AUC) of from about 10 to about 8000 day·μg/mL, about 10 to about 7000 day·μg/mL, about 10 to about 6000 day·μg/mL, about 10 to about 5000 day·μg/mL, about 10 to about 4000 day·μg/mL, about 10 to about 3000 day·μg/mL, about 10 to about 2500 day·μg/mL, about 10 to about 1000 day·μg/mL, about 10 to about 1500 day·μg/mL, about 10 to about 1000 day·μg/mL, about 10 to about 750 day·μg/mL, about 10 to about 500 day·μg/mL, about 10 to about 400 day·μg/mL, about 10 to about 300 day·μg/mL, about 10 to about 200 day·μg/mL, about 10 to about 100 day·μg/mL, about 10 to about 50 day·μg/mL, about 10 to about 25 day·μg/mL, about 25 to about 8000 day·μg/mL, about 25 to about 7000 day·μg/mL, about 25 to about 6000 day·μg/mL, about 25 to about 5000 day·μg/mL, about 25 to about 4000 day·μg/mL, about 25 to about 3000 day·μg/mL, about 25 to about 2500 day·μg/mL, about 25 to about 1000 day·μg/mL, about 25 to about 1500 day·μg/mL, about 25 to about 1000 day·μg/mL, about 25 to about 750 day·μg/mL, about 25 to about 500 day·μg/mL, about 25 to about 400 day·μg/mL, about 25 to about 300 day·μg/mL, about 25 to about 200 day·μg/mL, about 25 to about 100 day·μg/mL, about 25 to about 50 day·μg/mL, about 50 to about 8000 day·μg/mL, about 50 to about 7000 day·μg/mL, about 50 to about 6000 day·μg/mL, about 50 to about 5000 day·μg/mL, about 50 to about 4000 day·μg/mL, about 50 to about 3000 day·μg/mL, about 50 to about 2500 day·μg/mL, about 50 to about 2000 day·μg/mL, about 50 to about 1500 day·μg/mL, about 50 to about 1000 day·μg/mL, about 50 to about 750 day·μg/mL, about 50 to about 500 day·μg/mL, about 50 to about 400 day·μg/mL, about 50 to about 300 day·μg/mL, about 50 to about 200 day·μg/mL, about 50 to about 100 day·μg/mL, about 100 to about 8000 day·μg/mL, about 100 to about 7000 day·μg/mL, about 100 to about 6000 day·μg/mL, about 100 to about 5000 day·μg/mL, about 100 to about 4000 day·μg/mL, about 100 to about 3000 day·μg/mL, about 100 to about 2500 day·μg/mL, about 100 to about 1000 day·μg/mL, about 100 to about 1500 day·μg/mL, about 100 to about 1000 day·μg/mL, about 100 to about 750 day·μg/mL, about 100 to about 500 day·μg/mL, about 100 to about 400 day·μg/mL, about 100 to about 300 day·μg/mL, about 100 to about 200 day·μg/mL, about 200 to about 8000 day·μg/mL, about 200 to about 7000 day·μg/mL, about 200 to about 6000 day·μg/mL, about 200 to about 5000 day·μg/mL, about 200 to about 4000 day·μg/mL, about 200 to about 3000 day·μg/mL, about 200 to about 2000 day·μg/mL, about 200 to about 1000 day·μg/mL, about 200 to about 1500 day·μg/mL, about 200 to about 1000 day·μg/mL, about 200 to about 750 day·μg/mL, about 200 to about 500 day·μg/mL, about 200 to about 400 day·μg/mL, about 200 to about 300 day·μg/mL, about 300 to about 8000 day·μg/mL, about 300 to about 7000 day·μg/mL, about 300 to about 6000 day·μg/mL, about 300 to about 5000 day·μg/mL, about 300 to about 4000 day·μg/mL, about 300 to about 3000 day·μg/mL, about 300 to about 2500 day·μg/mL, about 300 to about 2000 day·μg/mL, about 300 to about 1500 day·μg/mL, about 300 to about 1000 day·μg/mL, about 300 to about 750 day·μg/mL, about 300 to about 500 day·μg/mL, about 300 to about 400 day·μg/mL, about 400 to about 8000 day·μg/mL, about 400 to about 7000 day·μg/mL, about 400 to about 6000 day·μg/mL, about 400 to about 5000 day·μg/mL, about 400 to about 4000 day·μg/mL, about 400 to about 3000 day·μg/mL, about 400 to about 2500 day·μg/mL, about 400 to about 2000 day·μg/mL, about 400 to about 1500 day·μg/mL, about 400 to about 1000 day·μg/mL, about 400 to about 750 day·μg/mL, about 400 to about 500 day·μg/mL, about 500 to about 8000 day·μg/mL, about 500 to about 7000 day·μg/mL, about 500 to about 6000 day·μg/mL, about 500 to about 5000 day·μg/mL, about 500 to about 4000 day·μg/mL, about 500 to about 3000 day·μg/mL, about 500 to about 2500 day·μg/mL, about 500 to about 2000 day·μg/mL, about 500 to about 1500 day·μg/mL, about 500 to about 1000 day·μg/mL, about 500 to about 750 day·μg/mL, about 750 to about 8000 day·μg/mL, about 750 to about 7000 day·μg/mL, about 750 to about 6000 day·μg/mL, about 750 to about 5000 day·μg/mL, about 750 to about 4000 day·μg/mL, about 750 to about 3000 day·μg/mL, about 750 to about 2500 day·μg/mL, about 750 to about 2000 day·μg/mL, about 750 to about 1500 day·μg/mL, about 750 to about 1000 day·μg/mL, about 1000 to about 8000 day·μg/mL, about 1000 to about 7000 day·μg/mL, about 1000 to about 6000 day·μg/mL, about 1000 to about 5000 day·μg/mL, about 1000 to about 4000 day·μg/mL, about 1000 to about 3000 day·μg/mL, about 1000 to about 2500 day·μg/mL, about 1000 to about 2000 day·μg/mL, about 1000 to about 1500 day·μg/mL, about 1500 to about 8000 day·μg/mL, about 1500 to about 7000 day·μg/mL, about 1500 to about 6000 day·μg/mL, about 1500 to about 5000 day·μg/mL, about 1500 to about 4000 day·μg/mL, about 1500 to about 3000 day·μg/mL, about 1500 to about 2500 day·μg/mL, about 1500 to about 2000 day·μg/mL, about 2000 to about 8000 day·μg/mL, about 2000 to about 7000 day·μg/mL, about 2000 to about 6000 day·μg/mL, about 2000 to about 5000 day·μg/mL, about 2000 to about 4000 day·μg/mL, about 2000 to about 3000 day·μg/mL, about 2000 to about 2500 day·μg/mL, about 2500 to about 8000 day·μg/mL, about 2500 to about 7000 day·μg/mL, about 2500 to about 6000 day·μg/mL, about 2500 to about 5000 day·μg/mL, about 2500 to about 4000 day·μg/mL, about 2500 to about 3000 day·μg/mL, about 3000 to about 8000 day·μg/mL, about 3000 to about 7000 day·μg/mL, about 3000 to about 6000 day·μg/mL, about 3000 to about 5000 day·μg/mL, about 3000 to about 4000 day·μg/mL, about 4000 to about 8000 day·μg/mL, about 4000 to about 7000 day·μg/mL, about 4000 to about 6000 day·μg/mL, about 4000 to about 5000 day·μg/mL, about 5000 to about 8000 day·μg/mL, about 5000 to about 7000 day·μg/mL, about 5000 to about 6000 day·μg/mL, about 6000 to about 8000 day·μg/mL, about 6000 to about 7000 day·μg/mL, or about 7000 to about 8000 day·μg/mL. In some embodiments, after administration to a subject in need thereof, the self-assembled polypeptide complex has an AUC of at least 10 day·μg/mL, at least 25 day·μg/mL, at least 50 day·μg/mL, at least 100 day·μg/mL, at least 200 day·μg/mL, at least 300 day·μg/mL, at least 400 day·μg/mL, at least 500 day·μg/mL, at least 750 day·μg/mL, at least 1000 day·μg/mL, at least 1500 day·μg/mL, at least 2000 day·μg/mL, at least 2500 day·μg/mL, at least 3000 day·μg/mL, at least 4000 day·μg/mL, at least 5000 day·μg/mL, at least 6000 day·μg/mL, at least 7000 day·μg/mL, or at least 8000 day·μg/mL.

In some embodiments, a self-assembled polypeptide complex as disclosed herein has a similar bioavailability to that of reference IgG molecule. For example, in some embodiments, after administration to a subject in need thereof, the self-assembled polypeptide complex has a maximum concentration (Cₘₐₓ) of from about 10 μg/mL to about 750 mg/mL, about 25 μg/mL to about 750 mg/mL, about 50 μg/mL to about 750 mg/mL, about 75 μg/mL to about 750 mg/mL, about 100 μg/mL to about 750 mg/mL, about 250 μg/mL to about 750 mg/mL, about 500 μg/mL to about 750 mg/mL, about 750 μg/mL to about 750 mg/mL, about 1 mg/mL to about 750 mg/mL, about 10 mg/mL to about 750 mg/mL, about 25 mg/mL to about 750 mg/mL, about 50 mg/mL to about 750 mg/mL, about 75 mg/mL to about 750 mg/mL, about 100 mg/mL to about 750 mg/mL, about 250 mg/mL to about 750 mg/mL, about 500 mg/mL to about 750 mg/mL, about 10 μg/mL to about 500 mg/mL, about 25 μg/mL to about 500 mg/mL, about 50 μg/mL to about 500 mg/mL, about 75 μg/mL to about 500 mg/mL, about 100 μg/mL to about 500 mg/mL, about 250 μg/mL to about 500 mg/mL, about 500 μg/mL to about 500 mg/mL, about 750 μg/mL to about 500 mg/mL, about 1 mg/mL to about 500 mg/mL, about 10 mg/mL to about 500 mg/mL, about 25 mg/mL to about 500 mg/mL, about 50 mg/mL to about 500 mg/mL, about 75 mg/mL to about 500 mg/mL, about 100 mg/mL to about 500 mg/mL, about 250 mg/mL to about 500 mg/mL, about 10 μg/mL to about 250 mg/mL, about 25 μg/mL to about 250 mg/mL, about 50 μg/mL to about 250 mg/mL, about 75 μg/mL to about 250 mg/mL, about 100 μg/mL to about 250 mg/mL, about 250 μg/mL to about 250 mg/mL, about 500 μg/mL to about 250 mg/mL, about 750 μg/mL to about 250 mg/mL, about 1 mg/mL to about 250 mg/mL, about 10 mg/mL to about 250 mg/mL, about 25 mg/mL to about 250 mg/mL, about 50 mg/mL to about 250 mg/mL, about 75 mg/mL to about 250 mg/mL, about 100 mg/mL to about 250 mg/mL, about 10 μg/mL to about 100 mg/mL, about 25 μg/mL to about 100 mg/mL, about 50 μg/mL to about 100 mg/mL, about 75 μg/mL to about 100 mg/mL, about 100 μg/mL to about 100 mg/mL, about 250 μg/mL to about 100 mg/mL, about 500 μg/mL to about 100 mg/mL, about 750 μg/mL to about 100 mg/mL, about 1 mg/mL to about 100 mg/mL, about 10 mg/mL to about 100 mg/mL, about 25 mg/mL to about 100 mg/mL, about 50 mg/mL to about 100 mg/mL, about 75 mg/mL to about 100 mg/mL, about 10 μg/mL to about 75 mg/mL, about 25 μg/mL to about 75 mg/mL, about 50 μg/mL to about 75 mg/mL, about 75 μg/mL to about 75 mg/mL, about 100 μg/mL to about 75 mg/mL, about 250 μg/mL to about 75 mg/mL, about 500 μg/mL to about 75 mg/mL, about 750 μg/mL to about 75 mg/mL, about 1 mg/mL to about 75 mg/mL, about 10 mg/mL to about 75 mg/mL, about 25 mg/mL to about 75 mg/mL, about 50 mg/mL to about 75 mg/mL, about 10 μg/mL to about 50 mg/mL, about 25 μg/mL to about 50 mg/mL, about 50 μg/mL to about 50 mg/mL, about 75 μg/mL to about 50 mg/mL, about 100 μg/mL to about 50 mg/mL, about 250 μg/mL to about 50 mg/mL, about 500 μg/mL to about 50 mg/mL, about 750 μg/mL to about 50 mg/mL, about 1 mg/mL to about 50 mg/mL, about 10 mg/mL to about 50 mg/mL, about 25 mg/mL to about 50 mg/mL, about 10 μg/mL to about 25 mg/mL, about 25 μg/mL to about 25 mg/mL, about 50 μg/mL to about 25 mg/mL, about 75 μg/mL to about 25 mg/mL, about 100 μg/mL to about 25 mg/mL, about 250 μg/mL to about 25 mg/mL, about 500 μg/mL to about 25 mg/mL, about 750 μg/mL to about 25 mg/mL, about 1 mg/mL to about 25 mg/mL, about 10 mg/mL to about 25 mg/mL, about 10 μg/mL to about 10 mg/mL, about 25 μg/mL to about 10 mg/mL, about 50 μg/mL to about 10 mg/mL, about 75 μg/mL to about 10 mg/mL, about 100 μg/mL to about 10 mg/mL, about 250 μg/mL to about 10 mg/mL, about 500 μg/mL to about 10 mg/mL, about 750 μg/mL to about 10 mg/mL, about 1 mg/mL to about 10 mg/mL, about 10 μg/mL to about 1 mg/mL, about 25 μg/mL to about 1 mg/mL, about 50 μg/mL to about 1 mg/mL, about 75 μg/mL to about 1 mg/mL, about 100 μg/mL to about 1 mg/mL, about 250 μg/mL to about 1 mg/mL, about 500 μg/mL to about 1 mg/mL, about 750 μg/mL to about 1 mg/mL, about 10 μg/mL to about 750 μg/mL, about 25 μg/mL to about 750 μg/mL, about 50 μg/mL to about 750 μg/mL, about 75 μg/mL to about 750 μg/mL, about 100 μg/mL to about 750 μg/mL, about 250 μg/mL to about 750 μg/mL, about 500 μg/mL to about 750 μg/mL, about 10 μg/mL to about 500 μg/mL, about 25 μg/mL to about 500 μg/mL, about 50 μg/mL to about 500 μg/mL, about 75 μg/mL to about 500 μg/mL, about 100 μg/mL to about 500 μg/mL, about 250 μg/mL to about 500 μg/mL, about 10 μg/mL to about 250 μg/mL, about 25 μg/mL to about 250 μg/mL, about 50 μg/mL to about 250 μg/mL, about 75 μg/mL to about 250 μg/mL, about 100 μg/mL to about 250 μg/mL, about 10 μg/mL to about 100 μg/mL, about 25 μg/mL to about 100 μg/mL, about 50 μg/mL to about 100 μg/mL, about 75 μg/mL to about 100 μg/mL, about 10 μg/mL to about 75 μg/mL, about 25 μg/mL to about 75 μg/mL, about 50 μg/mL to about 75 μg/mL, about 10 μg/mL to about 50 μg/mL, about 25 μg/mL to about 50 μg/mL, or about 10 μg/mL to about 25 μg/mL. In some embodiments, after administration to a subject in need thereof, the self-assembled polypeptide complex has a maximum concentration (Cₘₐₓ) of at least 10 μg/mL, at least 25 μg/mL, at least 50 μg/mL, at least 100 μg/mL, at least 250 μg/mL, at least 500 μg/mL, at least 750 μg/mL, at least 1 mg/mL, at least 10 mg/mL, at least 25 mg/mL, at least 50 mg/mL, at least 75 mg/mL, at least 100 mg/mL, at least 250 mg/mL, at least 500 mg/mL, or at least 750 mg/mL when administered to a subject in need thereof.

### Functional Effects

In certain embodiments, a provided self-assembled polypeptide complex is capable of antibody-dependent cellular phagocytosis (ADCP). In some embodiments, the ADCP is induced at a level of at least 20%, at least 25%, at least 30%, at least 35%, at least 40%, at least 45%, at least 50%, at least 55%, or at least 60% internalization of target. Methods of measuring ADCP are known in the art and include, for example, in vitro assays that utilize a macrophage cell line and a target.

### C. Methods of Treatment

In one aspect, provided are methods that may be useful for treating, ameliorating, or preventing a disease or a condition (e.g., an infectious disease, cancer, or an autoimmune disease), generally comprising a step of administering a composition comprising a self-assembled polypeptide complex of the present disclosure to a subject.

In some embodiments, the subject is a mammal, e.g., a human.

Compositions for administration to subjects generally comprise a self-assembled polypeptide complex as disclosed herein. In some embodiments, such compositions further comprise a pharmaceutically acceptable excipient.

Compositions may be formulated for administration for any of a variety of routes of administration, including systemic routes (e.g., oral, intravenous, intraperitoneal, subcutaneous, or intramuscular administration).

### EXAMPLES

### Example 1. Expression and Analysis of Representative Multabodies

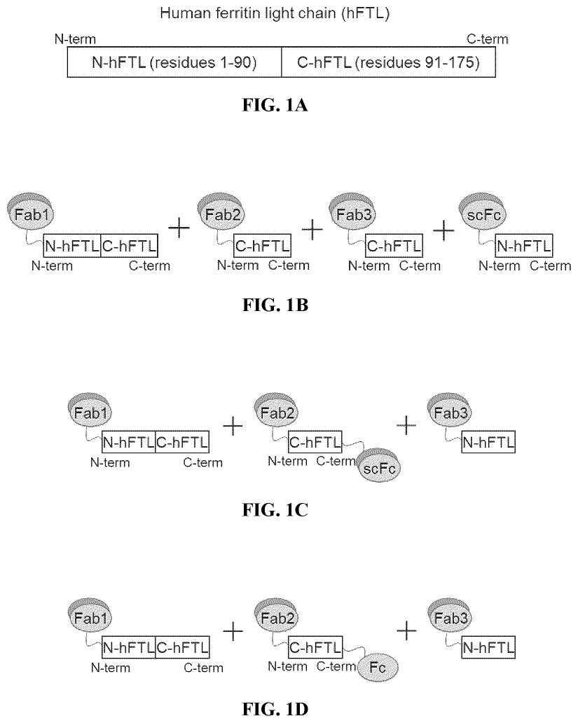

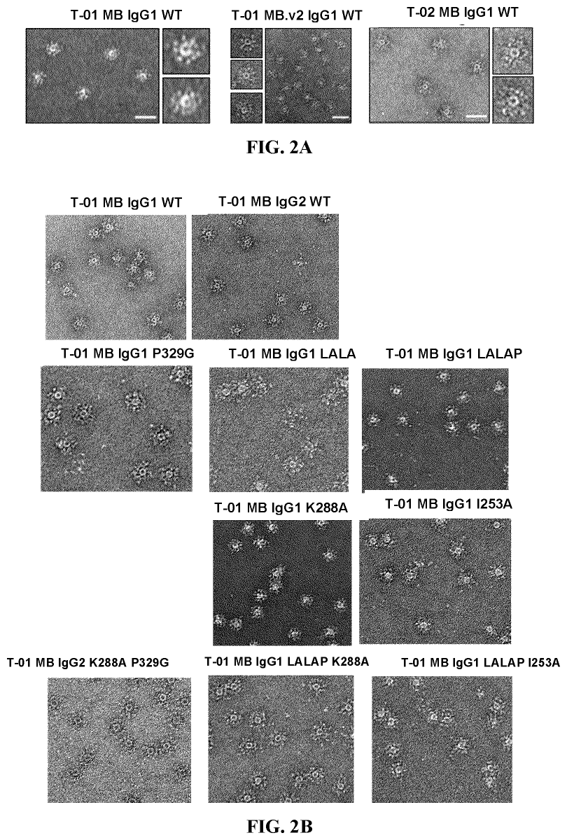

In this Example, Multabodies (MBs) were formed by fusion proteins generated by fusing together a human ferritin light chain (hFTL, SEQ ID NO:1), N-terminal fragment (residues 1-90) of hFTL (N_hFTL, SEQ ID NO:2), or C-terminal fragment (residues 91-175) of hFTL (C_hFTL, SEQ ID NO:3) with a single-chain Fab (scFab) and/or a fragment crystallizable region (Fc) or single chain Fc-dimer (scFc), via a linker, such as a (Glyn-Ser)ₘ peptide linker as described herein. The different formats that were generated are illustrated in FIG. 1.

T-01 MB: genes encoding fusion proteins (1) scFab of HIV-neutralizing antibody PGDM1400 fused to the N-terminus of hFTL (PGDM1400-hFTL, SEQ ID NO:27), (2) scFc fused to the N-terminus of N_hFTL (scFc-N_hFTL, SEQ ID NO:34), (3) scFab of HIV-neutralizing antibody N49P7 fused to the N-terminus of C_hFTL (N49P7-C_hFTL, SEQ ID NO:28), and (4) scFab of HIV-neutralizing antibody 10E8v4 fused to the N-terminus of C_hFTL (10E8v4-C_hFTL, SEQ ID NO:30) were prepared, mixed at a molar ratio of 4:2:1:1 and transiently transfected into HEK 293F cells for the production and formation of T-01 MB See, FIG. 1B.

T-02 MB: genes encoding fusion proteins (1) PGDM1400-hFTL (SEQ ID NO:27), (2) scFc-N_hFTL (SEQ ID NO:34), (3) scFab of anti-CD4 antibody ibalizumab (iMab) fused to the N-terminus of C_hFTL (iMab-C_hFTL, SEQ ID NO:31), and (4) 10E8v4-C_hFTL (SEQ ID NO:30) were prepared, mixed at a molar ratio of 4:2:1:1, and transiently transfected into HEK 293F cells for the production and formation of T-02 MB. See, FIG. 1B.

T-01 MB.v2: genes encoding fusion proteins (1) PGDM1400-hFTL (SEQ ID NO:27), (2) scFab of N49P7 fused to the N-terminus of N_hFTL (N49P7-N_hFTL (SEQ ID NO:29), and (3) an Fc monomer fused to the C-terminus of 10E8-C_hFTL (10E8v4-C_hFTL-Fc, SEQ ID NO:32) were prepared, mixed at a molar ratio of 3:1:1, and transiently transfected into HEK 293F cells for the production and formation of T-01 MB.v2. See, FIG. 1D. (As described for “T-01 MB” above, 10E8-C_hFTL contains the scFab of HIV-neutralizing antibody 10E8v4 fused to the N-terminus of C_hFTL. Thus, the construct 10E8v4-C_hFTL-Fc (SEQ ID NO:32) contains a C-half ferritin with the Fab3 at the N-terminus of the C-half ferritin and an Fc monomer at the C-terminus of the C-half ferritin.)

The Multabodies described in this Example had wild-type (WT) Fc or engineered IgG1 Fc. Such an engineered IgG1 Fc contained any one or more mutations of L234A, L235A, K288A, 1253V, 1253A, P329G, M428L, N434S, or any combinations thereof (according to the EU numbering scheme). For example, T-01 MB IgG1 K288A had engineered IgG1 Fc with the K288A mutation; T-01 MB IgG1 μLALAP had engineered IgG1 Fc with mutations of L234A, L235A, and P329G; T-01 MB.v2 IgG1 μLS had engineered IgG1 Fc with mutations of M428L and N434S.

The Multabodies were purified using Protein A affinity chromatography, optionally followed by Protein L affinity chromatography. Fractions containing the Multabodies were concentrated and further purified by size-exclusion chromatography (SEC) in sodium phosphate buffer. After SEC purification, negative-stain electron microscopy (EM) and/or SEC with inline multi-angle light scattering (SEC-MALS) was used to evaluate the size the of the of the formed Multabodies.

### Example 2. Binding of Multabodies Towards Fc Receptors Determined by Biolayer Interferometry

The binding kinetics and affinity to Fc receptors—human Fcγ receptor I (hFcγRI), hFcγRIIa, and hFcγRIIb—of Multabodies comprising different Fc mutations were determined by biolayer interferometry (BLI) using an Octet RED96 BLI system (Pall ForteBio).

Briefly, His-tagged Fc receptors were loaded onto Ni-NTA biosensors to reach a signal response of 0.8 nm. The association rates were measured by transferring the loaded biosensors to wells containing serial dilutions of the test Multabodies (20-10-5-2.5-1.25-0.65 nM) or IgG1 control (250-125-62.5-31.2-15.6-7.8 nM), with a contact time of 180 s. The IgG1 control is a cocktail of PGDM1400, N49P7, and 10E8v4 antibodies, all with wild-type IgG1 backbone. In order to assess the potential of the Multabodies to undergo endosomal recycling, their bindings to the hFcRn/β2-microglobulin complex were measured at both physiological pH (7.4) and acidic pH (5.6).

Fc mutations of IgG1 backbone evaluated in Multabodies include: K288A, 1253V, and I253A that decrease antibody binding to FcRn; P329G, LALA (L234A, L235A), and LALAP (L234A, L235A and P329G) that decrease antibody binding to FcγRs; and combinations thereof (Numberings are according to the EU numbering scheme.)

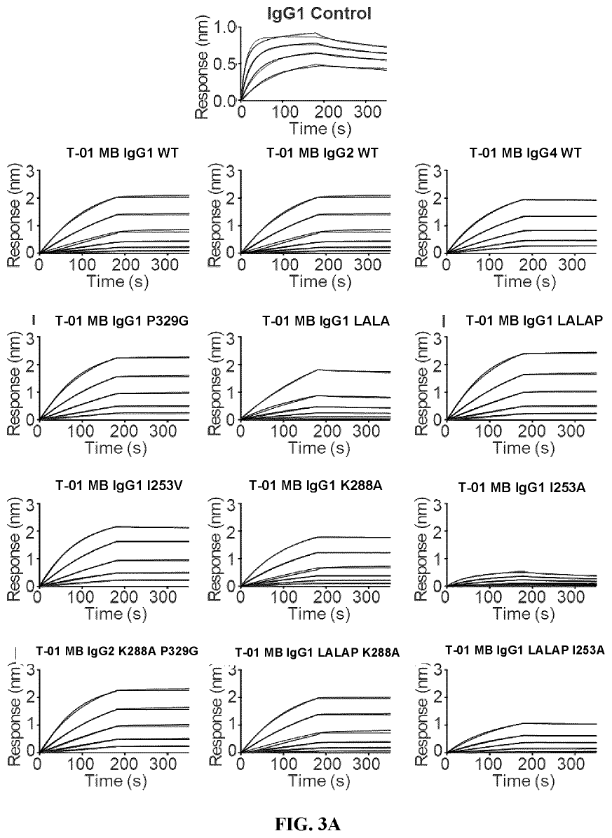

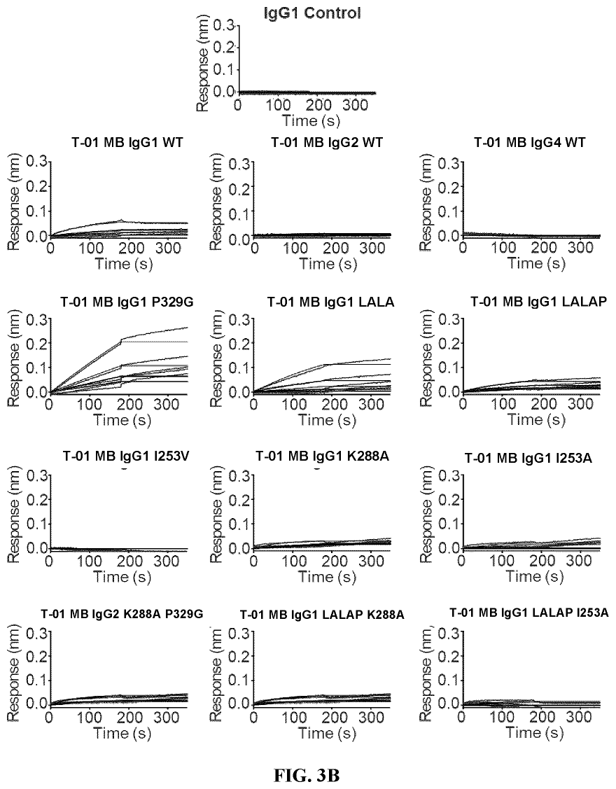

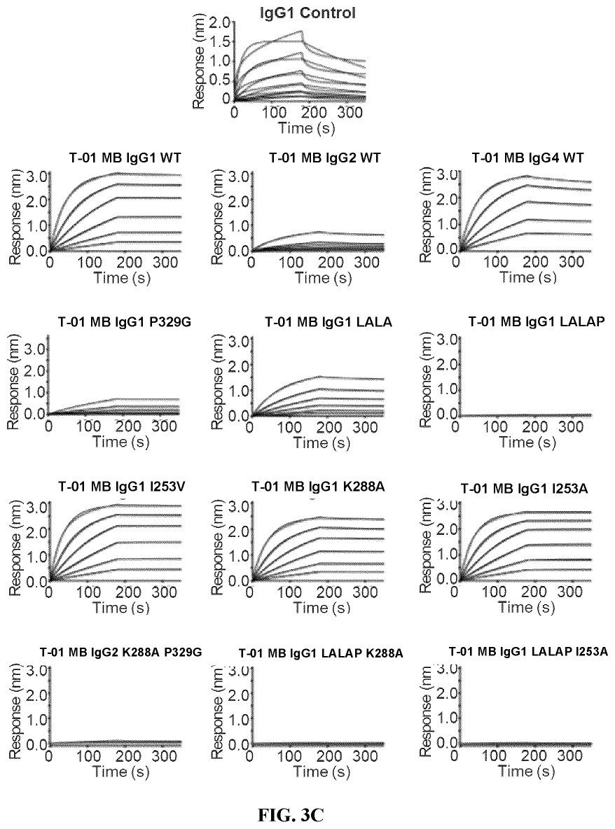

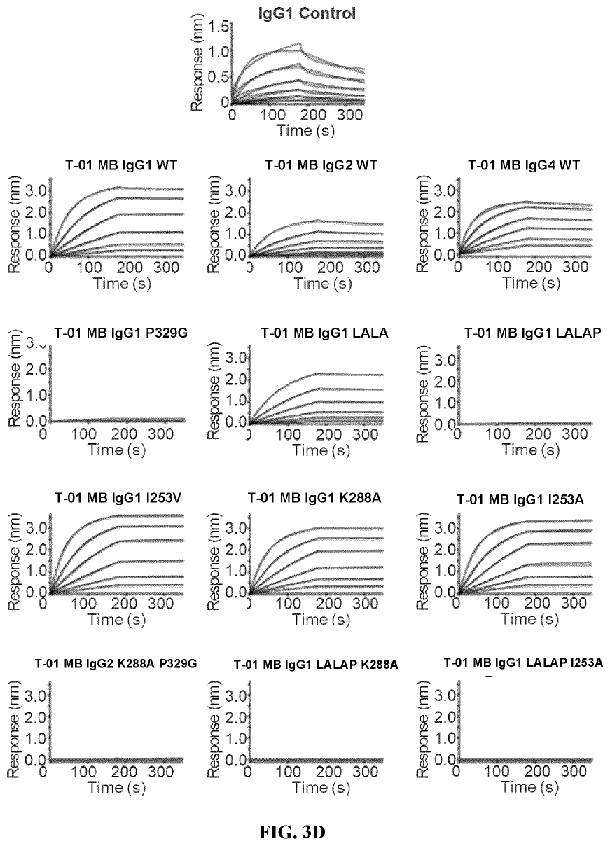

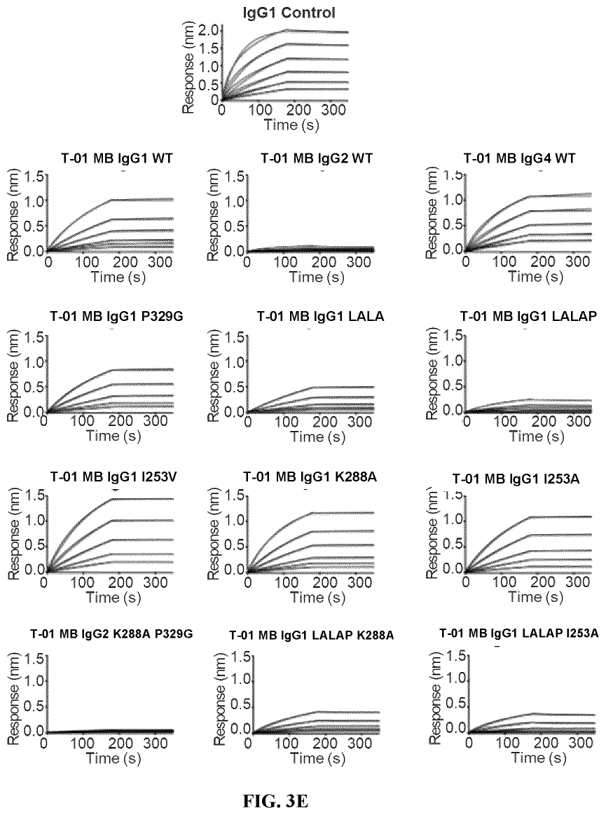

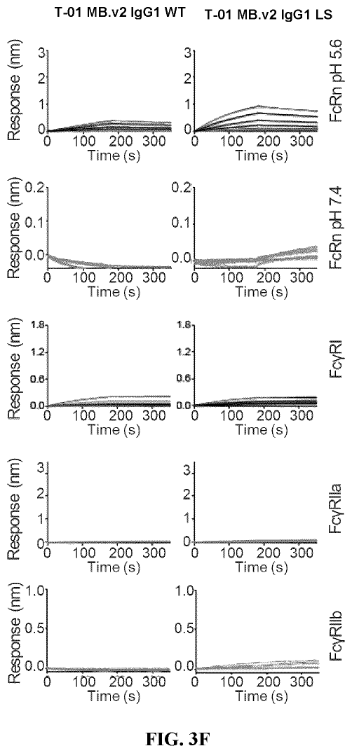

Representative examples for the relevant segment of the resulting sensorgrams are provided in FIGS. 3A, 3B, 3C, 3D, 3E, and 3F. The values determined for kₒₙ, k_{off}, and the resulting equilibrium dissociation constant (K_{D}) for the Multabodies are summarized in Tables 1 and 2.

At acidic pH (5.6), T-01 MB with wild-type IgG1 Fc binds to FcRn with over 1000-fold higher affinity compared to the IgG1 control; the same is also observed for T-01 MB with K288A, 1253V, P329G, LALA, LALAP, K288A+P329G, or K288A+LALAP IgG1 Fc mutations. T-01 MB with 1253A or 1253A+LALAP IgG1 Fc mutations have a similar binding ability to FcRn at pH 5.6 compared to the IgG1 control. At physiological pH (7.4), T-01 MB with wild-IgG1 Fc, P329G IgG1 Fc mutation, LALA IgG1 Fc mutations, or LALAP IgG1 Fc mutations show measurable binding to FcRn.

T-01 MB with wild-type IgG1 Fc binds to hFcγRI, hFcγRIIa, and hFcγRIIb with over 1000-fold higher affinity compared to the IgG1 control. Multabodies with P329G, LALA, or LALAP IgG1 Fc mutation(s) show reduced binding or non-binding to the tested Fcγ receptors.

T-01 MB.v2 with wild-type IgG1 Fc or with LS Fc mutations has a FcRn binding profile more similar to IgG1, with comparable binding ability to human FcRn at acidic pH and no binding at physiological pH. Further, T-01 MB.v2 with type IgG1 Fc or LS mutations shows reduced binding to the high affinity FcγRI and no binding to the low affinity Fcγ receptors tested, similar to T-01 MB containing the LALAP mutations.

Among the tested Fc mutations and mutation combinations, the I253A+LALAP IgG1 Fc mutation combination adjusts the Fc receptor binding profile of the Multabody (in T-01 MB format) to IgG1-like.

TABLE 1
Kinetic constants and affinities to FcRn of Multabodies determined by BLI

| | FcRn, pH 5.6 | | |
|---|---|---|---|
| Multabody | kₒₙ [M⁻¹ × s⁻¹] | k_{off} [s⁻¹] | K_{D} [M] |
| IgG1 control | 5.80E+05 | 1.07E−03 | 1.85E−09 |
| T-01 MB IgG1 WT* | 8.29E+05 | <1.0E−07 | <1.0E−12 |
| T-01 MB IgG2 WT | 9.19E+05 | <1.0E−07 | <1.0E−12 |
| T-01 MB IgG4 WT | 2.93E+05 | <1.0E−07 | <1.0E−12 |
| T-01 MB IgG1 I253A | 8.22E+05 | 2.01E−03 | 2.44E−09 |
| T-01 MB IgG1 I253V | 1.76E+06 | <1.0E−07 | <1.0E−12 |
| T-01 MB IgG1 K288A | 8.49E+05 | <1.0E−07 | <1.0E−12 |
| T-01 MB IgG1 P329G* | 9.08E+05 | <1.0E−07 | <1.0E−12 |
| T-01 MB IgG1 L234A L235A (LALA)* | 8.09E+05 | <1.0E−07 | <1.0E−12 |
| T-01 MB IgG1 LALA P329G (LALAP)* | 8.38E+05 | <1.0E−07 | <1.0E−12 |
| T-01 MB IgG2 K288A P329G | 8.71E+05 | <1.0E−07 | <1.0E−12 |
| T-01 MB IgG1 LALAP K288A | 8.42E+05 | <1.0E−07 | <1.0E−12 |
| T-01 MB IgG1 LALAP I253A | 7.39E+05 | 1.29E−03 | 1.75E−09 |
| T-01 MB.v2 IgG1 WT | 7.39E+05 | 1.79E−03 | 2.42E−09 |
| T-01 MB.v2 IgG1 M428L N434S (LS) | 2.94E+05 | 1.25E−03 | 4.23E−09 |

*Multabody shows residual binding to FcRn at pH 7.4

TABLE 2
Kinetic constants and affinities to Fcγ receptors of Multabodies determined by BLI

| | FcγRI | | | FcγRIIa | | | FcγRIIb | | |
|---|---|---|---|---|---|---|---|---|---|
| | kon | koff | KD | kon | koff | KD | kon | koff | KD |
| Multabody | [M−1 × s−1] | [s−1] | [M] | [M−1 × s−1] | [s−1] | [M] | [M−1 × s−1] | [s−1] | [M] |
| IgG1 control | 4.28E+04 | 1.28E−04 | 2.98E−09 | 2.39E+05 | 3.52E−03 | 1.48E−08 | 7.21E+04 | 2.54E−03 | 3.53E−08 |
| T-01 MB IgG1 | 6.72E+05 | <1.0E−07 | <1.0E−12 | 9.63E+05 | <1.0E−07 | <1.0E−12 | 1.19E+06 | <1.0E−07 | <1.0E−12 |
| WT | | | | | | | | | |
| T-01 MB IgG2 | 7.58E+05 | 1.54E−03 | 2.03E−09 | 4.33E+05 | 2.63E−04 | 6.06E−10 | 6.37E+05 | 1.03E−03 | 1.61E−09 |
| WT | | | | | | | | | |
| T-01 MB IgG4 | 8.35E+05 | <1.0E−07 | <1.0E−12 | 1.14E+06 | 4.45E−04 | 3.91E−10 | 1.37E+06 | 3.05E−04 | 2.23E−10 |
| WT | | | | | | | | | |
| T-01 MB IgG1 | 6.05E+05 | <1.0E−07 | <1.0E−12 | 7.63E+05 | <1.0E−07 | <1.0E−12 | 1.39E+06 | <1.0E−07 | <1.0E−12 |
| I253A | | | | | | | | | |
| T-01 MB IgG1 | 7.56E+05 | <1.0E−07 | <1.0E−12 | 1.09E+06 | <1.0E−07 | <1.0E−12 | 1.39E+06 | <1.0E−07 | <1.0E−12 |
| I253V | | | | | | | | | |
| T-01 MB IgG1 | 5.78E+05 | <1.0E−07 | <1.0E−12 | 1.07E+06 | <1.0E−07 | <1.0E−12 | 1.25E+06 | <1.0E−07 | <1.0E−12 |
| K288A | | | | | | | | | |
| T-01 MB IgG1 | 6.65E+05 | 2.49E−05 | 3.75E−11 | 2.22E+05 | 5.53E−05 | 2.49E−10 | — | — | No |
| P329G | | | | | | | | | binding |
| T-01 MB IgG1 | 5.58E+05 | 1.66E−05 | 2.97E−11 | 5.81E+05 | 1.21E−04 | 2.08E−10 | 6.49E+05 | 3.94E−04 | 6.07E−10 |
| LALA | | | | | | | | | |
| T-01 MB IgG1 | 6.39E+05 | 4.37E−04 | 6.84E−10 | — | — | No | — | — | No |
| LALAP | | | | | | binding | | | binding |
| T-01 MB IgG2 | 6.94E+05 | 9.13E−05 | 1.32E−10 | — | — | No | — | — | No |
| K288A P329G | | | | | | binding | | | binding |
| T-01 MB IgG1 | 6.01E+05 | 1.61E−04 | 2.69E−10 | — | — | No | — | — | No |
| LALAP K288A | | | | | | binding | | | binding |
| T-01 MB IgG1 | 8.53E+05 | 6.56E−04 | 7.68E−10 | — | — | No | — | — | No |
| LALAP I253A | | | | | | binding | | | binding |
| T-01 MB.v2 | 3.12E+05 | <1.0E−07 | <1.0E−12 | — | — | No | — | — | No |
| IgG1 WT | | | | | | binding | | | binding |
| T-01 MB.v2 | 4.31E+05 | <1.0E−07 | <1.0E−12 | — | — | No | — | — | No |
| IgGI LS | | | | | | binding | | | binding |

### Example 3. Target Binding of Multabodies Determined by Biolayer Interferometry

The binding kinetics and affinity to the respective target of PGDM1400, N49P7, 10E8v4, or iMab, as included as Fab in the Multabodies, were determined by BLI using an Octet RED96 BLI system (Pall ForteBio).

The experiments were performed similarly as described in Example 2, except that Hi-tagged targets—BG5050 SOSIP.664 D368R, gp120 subunit 93TH057, gp41 membrane-proximal external region (MPER), and soluble CD4 for PGDM1400, N49P7, 10E8v4, and iMab, respectively-were loaded onto Ni-NTA biosensors to reach a signal response of 0.8 nm. The loaded biosensors were then titrated with test Multabodies, PGDM1400, N49P7, 10E8v4, or iMab antibody (with a wild-type IgG1 backbone), or a cocktail (IgG1 control) of PGDM1400, N49P7, and 10E8v4 antibodies (all with a wild-type IgG1 backbone) at various concentrations.

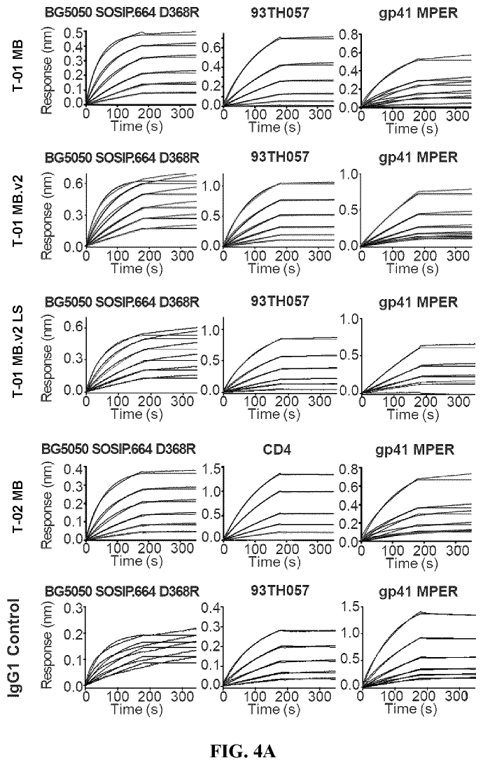

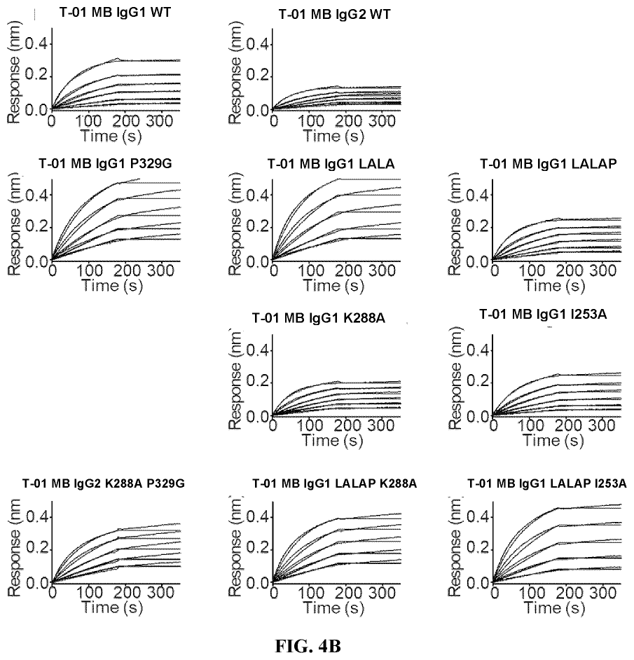

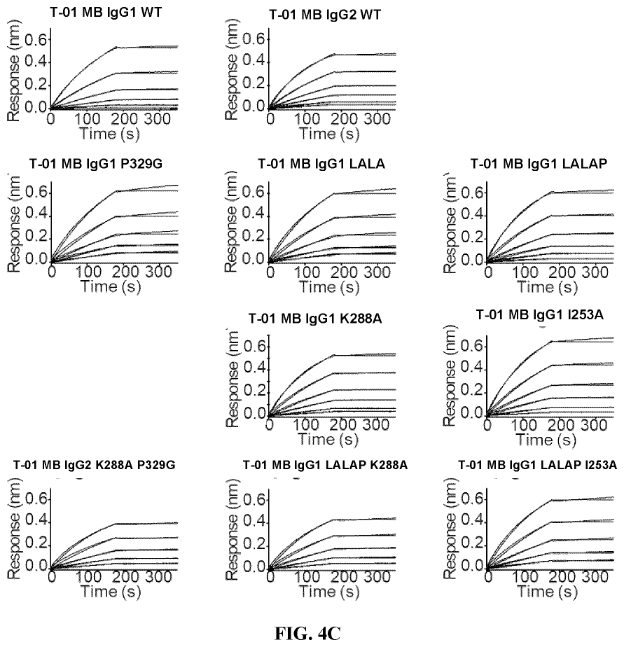

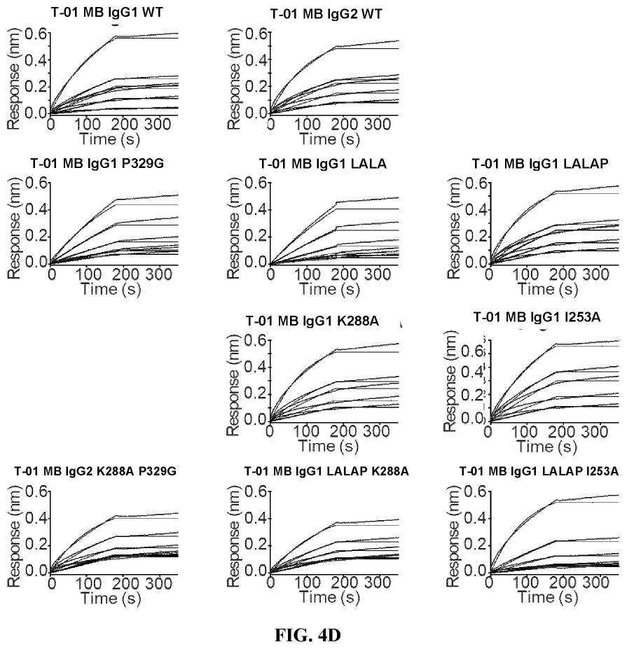

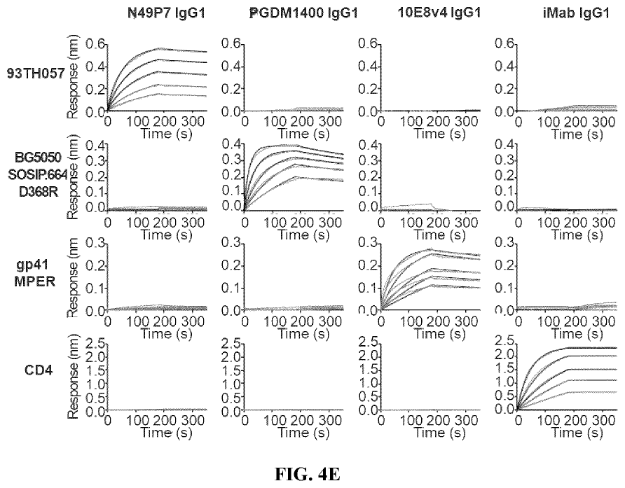

Representative examples for the relevant segment of the resulting sensorgrams are provided in FIGS. 4A, 4B, 4C, 4D), and 4E. The values determined for kₒₙ, k_{off}, and the resulting equilibrium dissociation constant (K_{D}) for the Multabodies are summarized in Tables 3 and 4.

TABLE 3
Kinetic constants and affinities to target binding of Multabodies determined by BLI

| | BG5050 SOSIP.664 D368R | | | 93TH057 | | | gp41 MPER | | |
|---|---|---|---|---|---|---|---|---|---|
| | kₒₙ | k_{off} | K_{D} | kₒₙ | k_{off} | K_{D} | kₒₙ | k_{off} | K_{D} |
| Multabody | [M⁻¹ × s⁻¹] | [s⁻¹] | [M] | [M⁻¹ × s⁻¹] | [s⁻¹] | [M] | [M⁻¹ × s⁻¹] | [s⁻¹] | [M] |
| IgG1 control | 1.00E+05 | 7.21E−04 | 7.20E−09 | 8.02E+04 | 1.08E−04 | 1.34E−09 | 4.16E+04 | 1.15E−04 | 2.77E−09 |
| T-01 MB | 8.03E+05 | <1.0E−07 | <1.0E−12 | 3.33E+05 | <1.0E−07 | <1.0E−12 | 3.87E+05 | <1.0E−07 | <1.0E−12 |
| IgG1 WT | | | | | | | | | |
| T-01 MB | 9.79E+05 | <1.0E−07 | <1.0E−12 | 4.65E+05 | <1.0E−07 | <1.0E−12 | 4.85E+05 | <1.0E−07 | <1.0E−12 |
| IgG2 WT | | | | | | | | | |
| T-01 MB | 7.36E+05 | <1.0E−07 | <1.0E−12 | 4.25E+05 | <1.0E−07 | <1.0E−12 | 4.21E+05 | <1.0E−07 | <1.0E−12 |
| IgG1 I253A | | | | | | | | | |
| T-01 MB | 1.04E+06 | <1.0E−07 | <1.0E−12 | 4.58E+05 | <1.0E−07 | <1.0E−12 | 5.00E+05 | <1.0E−07 | <1.0E−12 |
| IgG1 K288A | | | | | | | | | |
| T-01 MB | 9.78E+05 | <1.0E−07 | <1.0E−12 | 5.44E+05 | <1.0E−07 | <1.0E−12 | 2.42E+05 | <1.0E−07 | <1.0E−12 |
| IgG1 P329G | | | | | | | | | |
| T-01 MB | 1.18E+06 | <1.0E−07 | <1.0E−12 | 5.83E+05 | <1.0E−07 | <1.0E−12 | 1.35E+05 | <1.0E−07 | <1.0E−12 |
| IgG1 LALA | | | | | | | | | |
| T-01 MB | 9.39E+05 | <1.0E−07 | <1.0E−12 | 4.56E+05 | <1.0E−07 | <1.0E−12 | 5.25E+05 | <1.0E−07 | <1.0E−12 |
| IgG1 LALAP | | | | | | | | | |
| T-01 MB | 1.08E+06 | <1.0E−07 | <1.0E−12 | 6.51E+05 | <1.0E−07 | <1.0E−12 | 4.36E+05 | <1.0E−07 | <1.0E−12 |
| IgG2 K288A | | | | | | | | | |
| P329G | | | | | | | | | |
| T-01 MB | 1.25E+06 | <1.0E−07 | <1.0E−12 | 6.21E+05 | <1.0E−07 | <1.0E−12 | 3.36E+05 | <1.0E−07 | <1.0E−12 |
| IgG1 LALAP | | | | | | | | | |
| K288A | | | | | | | | | |
| T-01 MB | 1.11E+06 | <1.0E−07 | <1.0E−12 | 6.68E+05 | <1.0E−07 | <1.0E−12 | 2.25E+05 | <1.0E−07 | <1.0E−12 |
| IgG1 LALAP | | | | | | | | | |
| I253A | | | | | | | | | |
| T-01 MB.v2 | 1.08E+06 | <1.0E−07 | <1.0E−12 | 6.13E+05 | <1.0E−07 | <1.0E−12 | 1.83E+05 | <1.0E−07 | <1.0E−12 |
| IgG1 WT | | | | | | | | | |
| T-01 MB.v2 | 9.15E+05 | <1.0E−07 | <1.0E−12 | 4.98E+05 | <1.0E−07 | <1.0E−12 | 1.80E+05 | <1.0E−07 | <1.0E−12 |
| IgG1 LS | | | | | | | | | |

TABLE 4
Kinetic constants and affinities to target binding of Multabodies determined by BLI

| | | BG5050 SOSIP.664 | | | |
|---|---|---|---|---|---|
| | | D368R | 93TH057 | gp41 MPER | CD4 |
| T-01 MB | kₒₙ [M⁻¹ × s⁻¹] | 8.12E+05 | 3.67E+05 | 3.54E+05 | — |
| | k_{off} [s⁻¹] | <1.0E−07 | <1.0E−07 | <1.0E−07 | — |
| | K_{D} [M] | <1.0E−12 | <1.0E−12 | <1.0E−12 | No binding |
| T-02 MB | kₒₙ [M⁻¹ × s⁻¹] | 8.93E+05 | — | 4.16E+05 | 5.08E+05 |
| | k_{off} [s⁻¹] | <1.0E−07 | — | <1.0E−07 | <1.0E−07 |
| | K_{D} [M] | <1.0E−12 | No binding | <1.0E−12 | <1.0E−12 |
| PGDM1400 | kₒₙ [M⁻¹ × s⁻¹] | 6.90E+03 | — | — | — |
| | k_{off} [s⁻¹] | 2.62E−04 | — | — | — |
| | K_{D} [M] | 3.80E−08 | No binding | No binding | No binding |
| N49P7 | kₒₙ [M⁻¹ × s⁻¹] | — | 1.53E+05 | — | — |
| | k_{off} [s⁻¹] | — | 1.11E−03 | — | — |
| | K_{D} [M] | No binding | 1.39E−10 | No binding | No binding |
| 10E8v4 | kₒₙ [M⁻¹ × s⁻¹] | — | — | 6.51E+04 | — |
| | k_{off} [s⁻¹] | — | — | 1.90E−04 | — |
| | K_{D} [M] | No binding | No binding | 2.92E−09 | No binding |
| iMab | kₒₙ [M⁻¹ × s⁻¹] | — | — | — | 8.54E+04 |
| | k_{off} [s⁻¹] | — | — | — | <1.0E−07 |
| | K_{D} [M] | No binding | No binding | No binding | <1.0E−12 |

### Example 4. Pharmacokinetics of Multabodies in Mice

Analyses of the pharmacokinetics of Multabodies with wild-type Fc or engineered IgG1 Fc were performed in mice.

Test Multabodies or IgG1 control were injected into five CB17/Icr-Prkdc^{scid}/IcrIcoCrl immunodeficient (SCID) mice/group on day 0 at a dose of 5 mg/kg. The IgG1 control is a cocktail of PGDM1400, N49P7, and 10E8v4 antibodies, all with a wild-type IgG1 backbone. Serum samples were taken from day 1 and every two days for 9 days. On day 10, an additional dose of 5 mg/kg was administered and serum samples collected on day 11 and day 15.

In addition, Multabodies containing wild-type Fc, LALAP+1253A IgG1 Fc mutation combination, or M428L+N434S (LS) Fc mutation combination were subcutaneously injected in NOD/Shi-scid/IL-2Rγnull immunodeficient (NCG) mice (3 mice/group) at a single dose of 5 mg/kg. Blood samples were collected at multiple time points following the injection. IgG1 control was tested in parallel.

Levels of circulating Multabodies was assessed by ELISA. Briefly, 96-well plates were coated with 50 μL of His-tagged antigens recognized by the Fabs in the Multabodies at 0.5 μg/mL. Serum/blood samples were diluted and added to the wells. Bound agents were detected using HRP-Protein A as a secondary molecule. The chemiluminescence signal was quantified using a Microplate Reader. A calibration curve with standard protein dilutions was prepared.

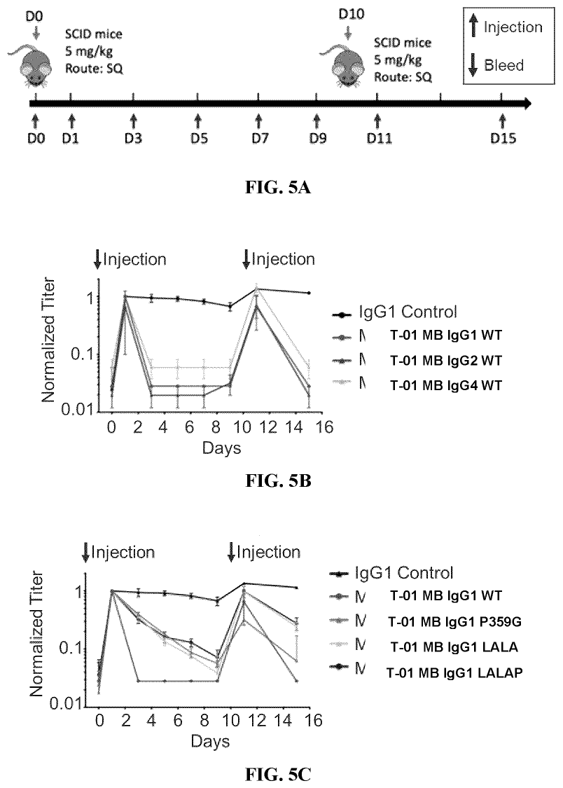

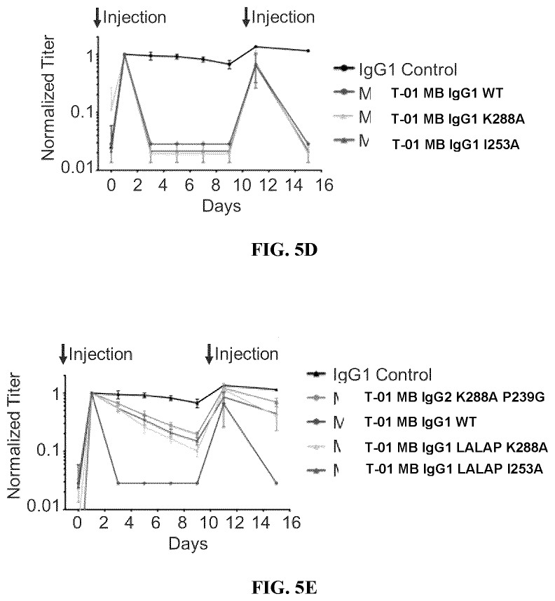

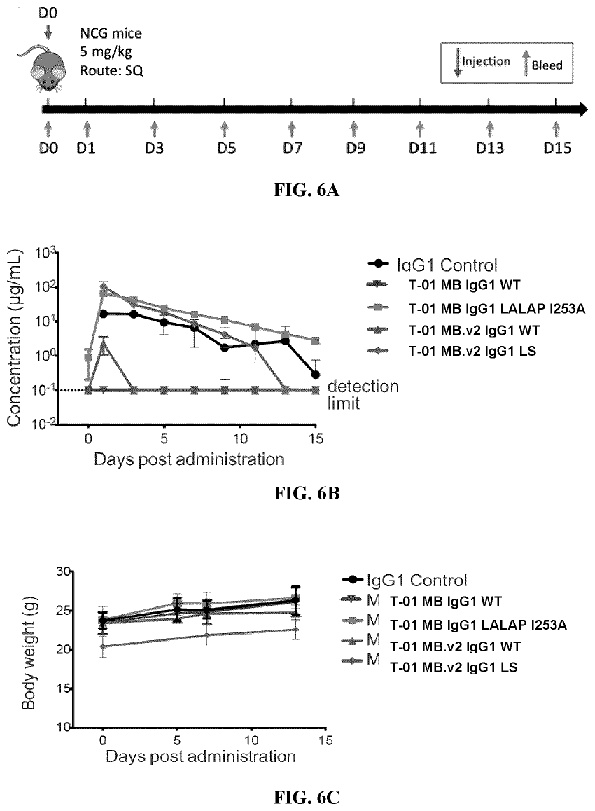

FIGS. 5B-5E and FIG. 6B show plots of the plasma concentration over time for tested Multabodies. LALAP+I235A, LALAP+K288A, and K288A+P329G mutation combinations were able to restore the serum level of T-01 MB to similar to the IgG1 control; LS mutation combination was able to restore the serum level of T-01 MB.v2 to similar to the IgG1 control. Accordingly, T-01 MB IgG1 μLALAP I235A, T-01 MB IgG1 μLALAP K288A, T-01 MB IgG1 K288A P329G, and T-01 MB.v2 IgG1 μLS display antibody-like pharmacokinetics or favorable pharmacokinetic profiles.

### Example 5. Assessment of Multabody-Induced Antibody-Dependent Cellular Phagocytosis

The potential of selected Multabodies to induce antibody-dependent cellular phagocytosis (ADCP) was assessed using THP-1 cell line.

Red fluorescent FluoSpheres NeutrAvidin microspheres were coated with biotinylated 93TH057 gp120 (antigen of N49P7) and incubated with the T-01 MB IgG1 LALAP I253A or T-01 MB.v2 IgG1 μLS or an IgG1 cocktail of PGDM1400, N49P7, and 10E8v4 IgG1 antibodies at various concentrations for 2 h at 37° C., followed by the addition of 200 μL THP-1 cells at 5×10⁴ cells/well. After 16 h, cells were pelleted and washed with PBS. The viability of cells was determined using Live/Dead Fixable Violet Stain. Cells washed with PBS were fixed with 2% paraformaldehyde for 20 min at room temperature, pelleted, and washed once with FACS buffer (PBS+10% FBS, 0.5 mM EDTA). Cells were subsequently analyzed using a BD LSR II Flow Cytometer, and data analyzed using FlowJo. IgG1 and Multabody controls with no affinity for 93TH057 gp120 were tested in parallel; FcR binding inhibitor antibody (Invitrogen, 14-9161-73) was used to block Fc-mediated internalization.

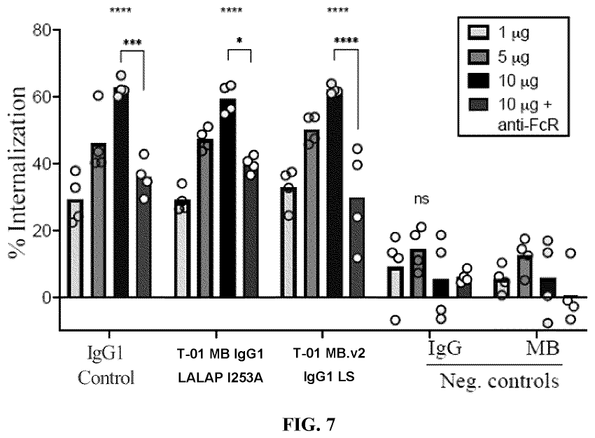

The results are depicted in FIG. 7. The phagocytosis was quantified and represented as the percentage increase in the internalization of 93TH057-coated microspheres compared to non-coated microspheres. T-01 MB.v2 IgG1 μLALAP I253A induces a dose-dependent ADCP, comparable to that of the IgG1 control, despite of the low binding to FcγRI (see Example 2).

### Example 6. Assessment of Multabody-Mediated Neutralization of HIV-1

The ability of selected Multabodies to neutralize HIV-1 was assessed using the TZM-bl assay, which measures HIV-1 neutralization as a function of reductions in HIV-1 Tat-regulated firefly luciferase (Luc) reporter gene expression after a single round of infection with Env-pseudotyped viruses.

Briefly, HIV-1 pseudotyped viruses were generated by co-transfection of 293T cells with the HIV-1 subtype B backbone NL4-3.Luc.R⁻E plasmid, the plasmid encoding the full-length Env clone. Test Multabodies, antibodies of the Fabs included in Multabodies, IgG1 control-1 (a cocktail of PGDM1400, N49P7, and 10E8v4 IgG1 antibodies), IgG1 control-2 (a cocktail of PGDM1400, iMab, and 10E8v4 IgG1 antibodies), or N6/PGDM1400x10 E8v4 trispecific antibody (directed to the CD4bs, V1V2 apex, and MPER binding sites) were incubated with a 10-15% tissue culture infectious dose of pseudovirus for 1 h at 37° C. prior to a 44-72 h incubation with the cells transfected with the pseudotyped viruses. Virus neutralization was monitored by adding Britelite plus reagent (PerkinElmer) to the cells and measuring luminescence in relative light units (RLUs) using a Synergy Neo2 Multi-Mode Assay Microplate Reader. Test articles were assayed against a single pseudovirus or a panel of 14 or 25 pseudoviruses (14- or 25-PsV panel). The 25-PsV panel included the strains in the 14-PsV panel, with the addition of 11 HIV-1 strains highly resistant to PDGM1400 in the 14-PsV panel. Thus, the 25-PsV panel contains 56% of PsV variants resistant (cutoff IC₅₀ set at 10 μg/mL) to PDGM1400 IgG neutralization.

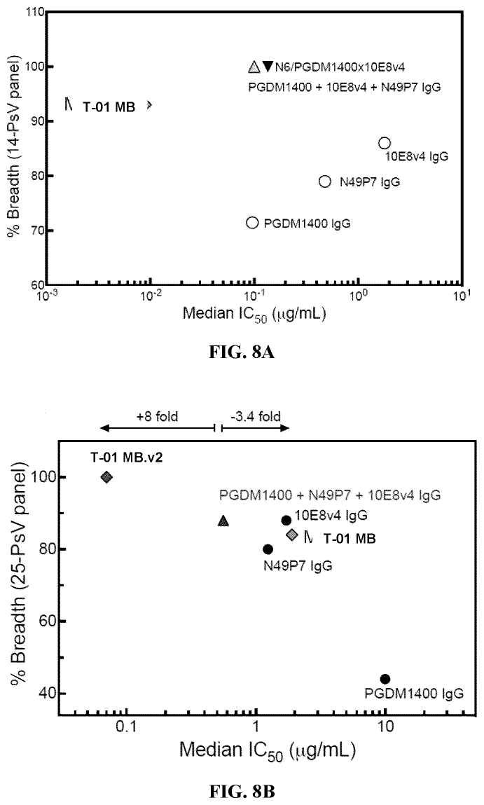

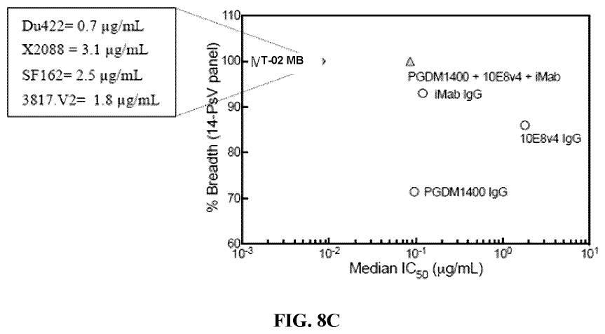

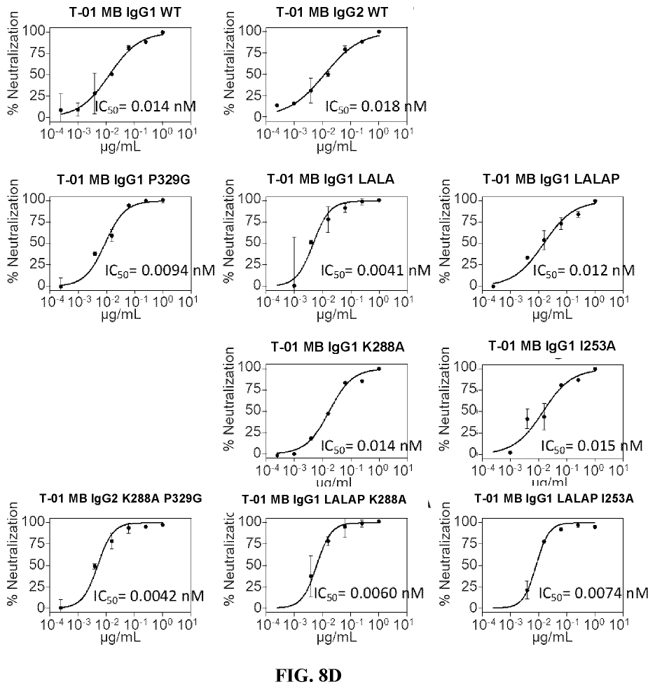

Exemplary results are shown in FIGS. 8A, 8B, 8C, and 8D, and the values determined for IC₅₀ and breadth of neutralization are summarized in Tables 5 and 6. The Multabodies display a decrease of approximately one and two orders of magnitude in the median IC₅₀ values compared to the IC₅₀ values of the IgG cocktails and the tri-specific antibody, respectively. T-01 MB.v2, in which the neutralization profile of antibodies N49P7 and 10E8v4 were more dominant than in other multabodies, achieved 100% neutralization in the 25-PsV panel.

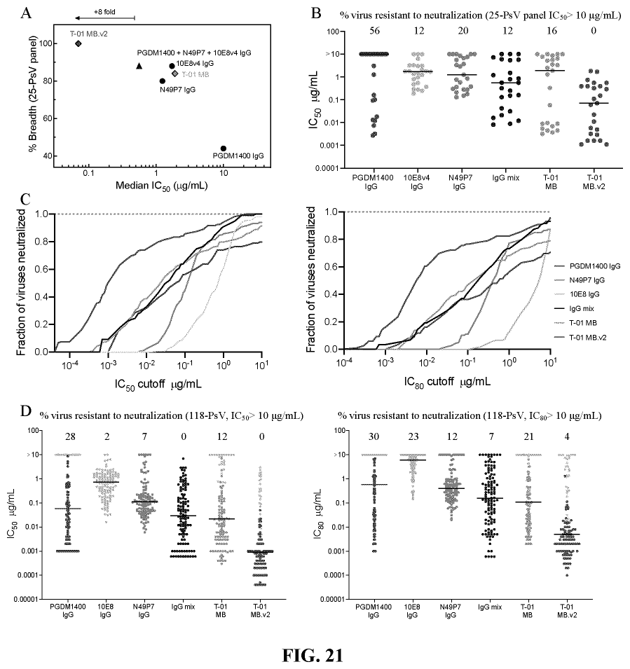

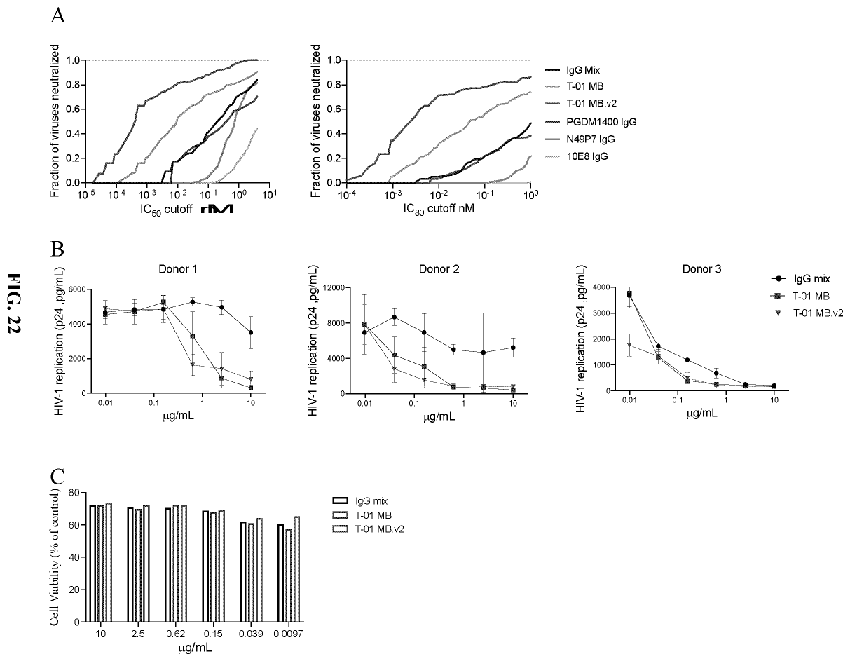

When tested against an extended multiclade panel of 118 PsV, T-01 MB.v2 matched the pan-neutralization breadth of the corresponding IgG cocktail (100% virus coverage, cutoff IC₅₀ set at 10 μg/mL), yet displayed a remarkable neutralization potency (FIG. 21C-D, FIG. 22A and Table 9). Specifically, the IgG cocktail and T-01 MB were only able to neutralize 9% and 8% of the PsV with an IC₅₀ value of 0.001 μg/mL, respectively, while in the case of T-01 MB.v2, 50% of the PsVs were still neutralized with an IC₅₀ value of only 0.001 μg/mL (FIG. 21C). Remarkably, Multabodies achieved a median IC₅₀ value of only 0.0009 μg/mL (0.4 pM) and hence achieved pan-neutralization 32- and 490-fold more potently in mass and molarity, respectively, compared to the IgG cocktail (FIG. 21D). In addition, the IC₈₀ of T-01 MB.v2 confirmed its superior neutralization propensity over both the individual IgGs and the IgG cocktail, neutralizing 96% of all viral strains tested with a median IC₈₀ value of 0.005 μg/mL (2.2 pM) (FIG. 21C-D, FIG. 22A and Table 9). Importantly, Multabodies also blocked infection of primary peripheral blood mononuclear cells (PBMCs) with the replication-competent CXCR4-tropic HIV-1 IIIB strain (FIG. 22B), showing enhanced potency over the matched IgG mix, and without any impact on cell viability (FIG. 22C).

TABLE 5
Neutralization of HIV by Multabodies

| | 14-PsV Panel | | 25-PsV Panel | |
|---|---|---|---|---|
| | Median IC₅₀ | % Virus | Median IC₅₀ | % Virus |
| Multabody | [μg/mL] | Neutralized | [μg/mL] | Neutralized |
| PGDM1400 IgG1 | 0.096 | 71 | 10 | 44 |
| 10E8.v4 IgG1 | 1.8 | 86 | 1.72 | 88 |
| N49P7 IgG1 | 0.48 | 79 | 1.24 | 80 |
| iMab IgG1 | 0.12 | 93 | — | — |
| IgG1 Control-1 | 0.086 | 100 | 0.56 | 88 |
| IgG1 Control-2 | 0.10 | 100 | — | — |
| N6/PGDM1400x10E8v4 | 0.13 | 100 | — | — |
| T-01 MB (IgG1) | 0.009 | 93 | 1.9 | 84 |
| T-01 MB.v2 (IgG1) | 0.0049 | 100 | 0.07 | 100 |
| T-02 MB (IgG1) | 0.008 | 100 | — | — |

TABLE 6
Neutralization of HIV by Multabodies

| Multabody | IC₅₀ |
|---|---|
| T-01 MB IgG1 | 0.014 nM |
| T-01 MB IgG2 | 0.018 nM |
| T-01 MB IgG1 P329G | 0.0094 nM |
| T-01 MB IgGI LALA | 0.0041 nM |
| T-01 MB IgGI LALAP | 0.012 nM |
| T-01 MB IgG1 K288A | 0.014 nM |
| T-01 MB IgG1 I253A | 0.015 nM |
| T-01 MB IgG1 K288A P329G | 0.0042 nM |
| T-01 MB IgGI LALAP K288A | 0.0060 nM |
| T-01 MB IgGI LALAP I253A | 0.0074 nM |

### Example 7. Assessment of Inhibition of HIV-1 Infection by Multabody

The ability of selected Multabodies to inhibit HIV-1 infection was assessed using human peripheral blood mononuclear cells (PBMCs).

Briefly, PBMCs were obtained from three healthy blood donors and activated with phytohemagglutinin (PHA) in the presence of recombinant human IL-2 in complete RPMI medium supplemented with 10% fetal bovine serum (FBS) for 72 h prior to HIV-1 infection. The laboratory CXCR4-tropic HIV-1 isolate IIIB was incubated with testing Multabodies or an IgG1 control for 1 h at room temperature and then added to the activated PBMCs in triplicates. The IgG1 control is a cocktail of PGDM1400, N49P7, and 10E8v4 antibodies, all with an wild-type IgG1 backbone. Infected cells were cultured in the presence or absence of the testing Multabodies or antibody controls at doses ranging from 0.01-10 ug/mL. The levels of HIV-1 replication were assessed at day 7 post-infection by measuring the extracellular release of p24 Gag protein in cell-free culture supernatants using a high-sensitivity AlphaLISA p24 detection kit on a BioTEK Synergy Plate Reader, according to the manufacturer's protocols. Cell viability was also assessed on day 7 of infection by fixing the cells in 2% PFA, and the absolute number of cells was counted by flow cytometry using a BD LSRFortessa (Becton Dickinson).

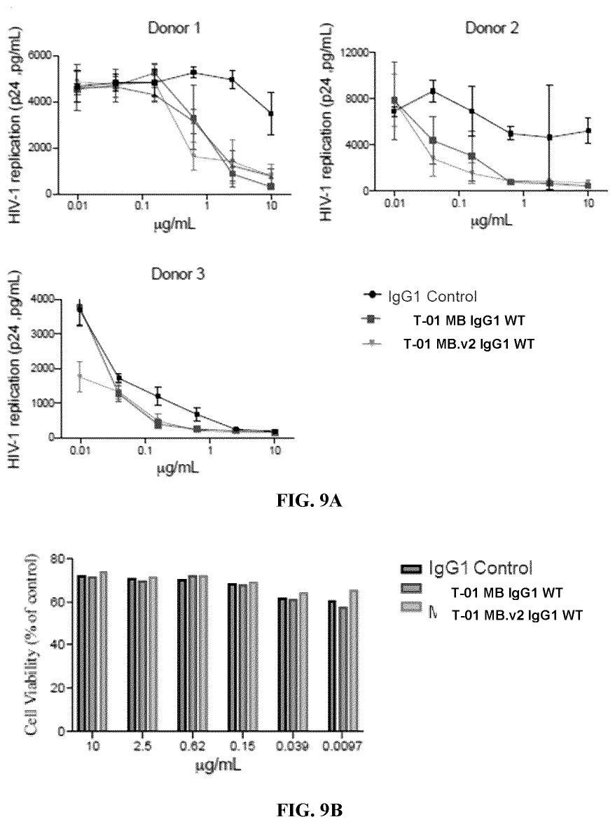

Exemplary results are shown in FIGS. 9A and 9B. The Multabodies (T-01 MB and T-01 MB.v2) were able to inhibit the infection of primary PBMCs by the replication-competent CXCR4-tropic HIV-1 isolate IIIB, with enhanced potency compared to the IgG1 control and without any impact on cell viability.

### Example 8. Characterization of Thermostability of Multabody

The melting temperature (Tₘ) and aggregation temperature (T_{agg}) of the Multabodies and reference molecules (parental antibodies PGDM1400, N49P7, 10E8v4, and iMab; PGDM1400x10 E8v4 trispecific antibody) were determined using a UNit system. Samples were concentrated to 1.0 mg/mL and subjected to a thermal ramp from 25 to 95° C. with 1° C. increments. Tₘ was obtained by measuring the barycentric mean fluorescence; T_{agg} was determined as the temperature at which 50% increase in the static light scattering at a 266 nm wavelength relative to baseline was observed. The average and the standard error of 3 independent measurements were calculated using the UNit analysis software. Table 7 summarizes the Tₘ and T_{agg}.

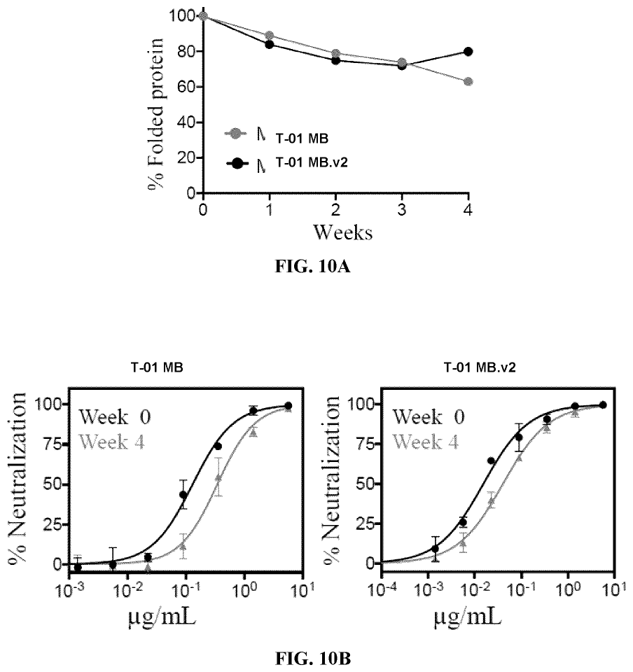

Stability of Multabodies was further analyzed under accelerated conditions. Samples were concentrated to 10 mg/mL and incubated at 40° C. for four weeks. Each week, the percentage of properly folded protein was calculated based on the soluble content from SEC. Multabodies were highly stable under these conditions, with over 70% of the sample remaining soluble for 30 days. (See. FIG. 10A).

Additionally, a sample before (week 0) and after (week 4) the incubation period was assessed in a PsV neutralization assay to compare the biological function of the molecules. Stability was further confirmed by only a modest loss in neutralization potency at week 4 in comparison to their potency at week 0. (See FIG. 10B).

Tested Multabodies have similar thermostability compared to the reference molecules and are stable for at least four weeks when stored at 40° C. with minimal loss in neutralization potency.

TABLE 7
Melting temperature (Tₘ) and aggregation
temperature (T_{agg}) of Multabodies

| Multabody | Tₘ [° C.] | T_{agg} [° C.] |
|---|---|---|
| PGDM1400 IgG1 | 53 | 67 |
| 10E8.v4 IgG1 | 69 | 72 |
| N49P7 IgG1 | 65 | 81 |
| iMab IgG1 | 68 | 76 |
| T-01 MB IgG1 WT | 63 | 68 |
| T-02 MB IgG1 WT | 66 | 69 |
| T-01 MB.v2 IgG1 WT | 65 | 68 |

### Example 9. Engineering Pan-HIV-1 Neutralization Potency Through Multi-Specific Antibody Avidity

### Abstract

Deep-mining of B-cell repertoires of HIV-1 infected individuals has resulted in the isolation of dozens of HIV-1 broadly neutralizing antibodies (bNAbs). Yet, it remains uncertain whether any such bNAbs alone are sufficiently broad and potent to deploy therapeutically. Here, we engineered HIV-1 bNAbs for their combination on a single multi-specific and avid molecule via direct genetic fusion of their Fab fragments to the human apoferritin light chain. The resulting molecule demonstrated a remarkable median IC₅₀ value of 0.0009 μg/mL and 100% neutralization coverage of a broad HIV-1 pseudovirus panel (118-isolates) at a 4 μg/mL cut-off—a 32-fold enhancement in viral neutralization potency compared to a cocktail of the corresponding HIV-1 bNAbs. Importantly, Fc incorporation on the molecule and engineering to modulate Fc receptor binding resulted in IgG-like bioavailability in vivo. This robust plug-and-play antibody design is relevant against indications where multi-specificity and avidity are leveraged simultaneously to mediate optimal biological activity.

The high genetic diversity of HIV-1 continues to be a major barrier to the development of therapeutics for prevention and treatment. Here, we describe the design of an antibody platform that allows assembly of a highly avid, multi-specific molecule that targets simultaneously the most conserved epitopes on the HIV-1 Envelope glycoprotein. The combined multi-valency and multi-specificity translates into extraordinary neutralization potency and pan-neutralization of HIV-1 strains, surpassing that of the most potent anti-HIV broadly neutralizing antibody cocktails.

### INTRODUCTION

Despite decades of research, no effective vaccine or cure exists for the human immunodeficiency virus type I (HIV-1). However, the fact that a small proportion of HIV-1 infected individuals develop antibodies with exceptional neutralization potency across circulating HIV-1 isolates highlights the potential for antibody-mediated control of HIV-1. Since the first-generation of broadly neutralizing antibodies (bNAbs) 2F5 (1), 4E10 (2, 3), 2G12 (4) and b12 (5, 6) were isolated, the number of bNAbs has dramatically increased due to implementation of new technologies such as Env-specific single B cell sorting (7-9), antibody cloning and high-throughput neutralization assays (10-13), and, more recently, proteomic deconvolution (14). Several HIV-1 bNAbs have now been described that primarily target six conserved sites on the trimeric HIV Envelope glycoprotein (Env), including the V1/V2 loops at the trimer apex, V3 loop glycans, the CD4 binding site (CD4bs), the gp120-g41 interface, the Env silent face and the membrane-proximal external region (MPER) (7, 9, 11-20).

Interest in bNAbs as therapeutic agents in the fight against HIV-1 arise from the potent antiviral activity observed in challenge studies in macaques (21-25) and humanized mice (26-29), and from the reduced viremia achieved in infected humans following infusion of bNAbs (30-34). In addition, antibodies possess key advantages in comparison to oral antiretroviral therapy (ART): they have longer circulating half-lives, and can form immune complexes that enhance host immunity to the virus. These observations have led to the clinical evaluation of antibody-based therapies to confer protection against HIV-1 acquisition through passive administration of bNAbs (35), and efforts to control and/or clear HIV-1 in infected individuals (31-33).

The recent Antibody Mediated Prevention (AMP) trials explored the ability of bNAb VRC01 to confer passive immunity against HIV-1. In these studies, antibody breadth and potency inferred from TZM-bl neutralization assays were proposed as effective predictors of antibody efficacy in humans. Specifically, an IC₈₀ value below 1 μg/ml was established as the potency threshold that a biotherapeutic needs to achieve in order to confer protection against a specific HIV-1 strain (35). VRC01 only met that threshold against 30% of HIV-1 strains in the trials and hence, failed to confer broad protection, highlighting a critical need for more potent and broadly acting molecules. While such breadth of coverage could be achieved by administration of multiple bNAbs, despite recent IgG engineering efforts (36-40), potency may still limit the therapeutic efficacy of antibody cocktails.

Here, we overcome the immense sequence diversity of HIV-1 with extraordinary neutralization potency by engineering the human apoferritin subunit to drive multimerization of three different HIV-1 bNAbs on a single molecule. The resulting MULTi-specific, multi-Affinity antiBODY (Multabody) was able to achieve pan-neutralization (100% virus coverage) with a median IC₅₀ value of 0.0009 μg/mL (0.4 pM). The Multabody design described herein represents a robust and powerful plug-and-play platform to multimerize antibodies in order to enhance their neutralization of HIV-1 across the broadest range of isolates.

### Materials and Methods

Expression and purification of Fab-only apoferritin multimers. Genes encoding the light chain of human apoferritin and the scFab-human apoferritin fusions were synthesized and cloned by GeneArt (Life Technologies) into the pHLsec expression vector. 200 mL of HEK 293F cells (Thermo Fisher Scientific) were seeded at a density of 0.8×10⁶ cells/mL in Freestyle expression media and incubated with 125 rpm oscillation at 37° C., 8% CO₂, and 70% humidity in a Multitron Pro shaker (Infors HT). Within 24 h of seeding, cells were transiently transfected using 50 μg of filtered DNA preincubated for 10 min at room temperature (RT) with the transfection reagent FectoPRO (Polyplus Transfections) at a 1:1 ratio. Plasmids encoding scFab-human apoferritin and human apoferritin were mixed at a ratio of 1:4, 1:1, 4:1 and 1:0. After 6-7 days, cell suspensions were harvested by centrifugation at 5000×g for 15 min and the supernatants filtered through a 0.22 μm Steritop filter (EMD Millipore). The particles were purified by affinity chromatography to the Fab and eluted after a wash. Fractions containing protein were pooled, concentrated and loaded onto a Superose 6 10/300 GL size exclusion column (GE Heathcare) in 20 mM sodium phosphate, pH 8.0, 150 mM NaCl.

Design, expression and purification of Multabodies. Genes encoding scFab and scFc fragments linked to half ferritin were generated by deletion of residues 1 to 90 (C-Ferritin) and 91 to 175 (N-Ferritin) of the light chain of human apoferritin. Furthermore, protein L binding specificity for iMab-C-Ferritin was disrupted by site-directed mutagenesis of alanine 12 of the antibody light chain to a proline residue (69). Transient transfection of T-01 MB in HEK 293F cells was obtained by mixing 66 μg of plasmids PGDM1400 scFab-human apoferritin: scFc-N-Ferritin: N49P7 scFab-C-Ferritin: 10E8v4 scFab-C-Ferritin in a 4:2:1:1 ratio. In the case of the T-02 MB, plasmid N49P7 scFab-C-Ferritin was substituted by iMab scFab-C-Ferritin. In the case of the T-01 MB.v2, 63 μg of plasmids PGDM1400 scFab-human apoferritin: N49P7 scFab-N-Ferritin: 10E8v4 scFab-C-Ferritin-Fc in a 3:1:1 ratio were used. The DNA mixture was filtered and incubated at RT with 60 μl of FectoPRO before adding to the cell culture. Based on the necessary hetero-oligomerization to drive self-assembly, purification of Multabodies with the four components was achieved by a two-step affinity purification: protein A HP column (GE Healthcare) with 20 mM Tris pH 8.0, 3 M MgCl₂ and 10% glycerol elution buffer (Fc binding) and protein L (GE Healthcare) (PGDM1400 binding, as 10E8 and N49P7 do not bind to Protein L, and iMab-protein L binding was disrupted by the A12P mutation). A buffer exchange step was performed between both affinity chromatography steps using a PD-10 desalting column (GE Healthcare). Fractions containing the protein were concentrated and further purified by gel filtration on a Superose 6 10/300 GL column (GE Healthcare) in 20 mM sodium phosphate pH 8.0, 150 mM NaCl.

Negative-stain electron microscopy. 3 μL of Multabody at a concentration of approximately 0.02 mg/mL was added to a carbon-coated copper grid for 30 s and stained with 3 μl of 2% uranyl formate. Staining excess was immediately removed from the grid using Whatman No. 1 filter paper and an additional 3 μl of 2% uranyl formate was added for 20 s. Grids were imaged using a field-emission FEI Tecnai F20 electron microscope operating at 200 kV and equipped with an Orius charge-coupled device (CCD) camera (Gatan Inc).

Biolayer interferometry. Binding kinetics measurements were conducted using an Octet RED96 BLI system (Pall ForteBio) in PBS pH 7.4, 0.01% BSA and 0.002% Tween. A unique His-tagged ligand for each of the Multabody components and Fc receptors was selected and loaded onto Ni-NTA biosensors to reach a signal response of 0.8 nm. Association rates were measured by transferring the loaded biosensors to wells containing serial dilutions of the Multabodies (10-5-2.5-1.25-0.65-0.32 nM) or IgGs (500-250-125-62.5-31.2-15.6 nM). Dissociation rates were measured by dipping the biosensors into buffer-containing wells. The duration of each of these two steps was 180 s. To achieve selective binding to PGDM1400, a D368R mutation in the CD4bs of the BG5050 SOSIP.664 trimer was introduced and, consequently, binding of N49P7 to this antigen was disrupted. Similarly, the gp120 subunit 93TH057, soluble CD4 and hFcRn in complex with P2-microglobulin were produced as the ligands for N49P7, iMab and Fc, respectively. Binding to 10E8 was tested using a His-tagged MPER peptide

(SEQ ID NO: 47)
(HHHHHHNEQELLELDKWASLWNWFNITNWLWYIKKKK,

purchased from GenScript). Recombinantly expressed hFcγRI and hFcγRIIa were used to measure binding affinities of the IgGs and Multabodies with effector function silencing mutations. Ni-NTA purification followed by size exclusion chromatography in 20 mM phosphate, pH 8.0, 150 mM NaCl buffer was used for purification of BG5050 SOSIP.664 D368R, CD4, 93TH057, hFcRn, hFcγRI and hFcγRIIa.

Size-exclusion chromatography in-line with multi-angle light scattering (SEC-MALS). A MiniDAWN TREOS and an Optilab T-rEX refractometer (Wyatt) were used in-line with an Agilent Technologies 1260 infinity II HPLC. 50 μg of 24-mer PGDM1400 scFab-ferritin fusion, T-01 MB and T-02 MB were loaded onto a Superose 6 10/300 (GE Healthcare) column in 20 mM sodium phosphate, pH 8.0, 150 mM NaCl. Data collection and analysis were performed using the ASTRA software (Wyatt).

Stability measurements. The melting temperature (Tₘ) and aggregation temperature (T_{agg}) of Multabodies, parental IgGs, the 12-mer homo-oligomeric Fabs and Fc and the N6/PGDM1400x10 E8v4 tri-specific antibody were determined using a UNit system (Unchained Labs). Tₘ was obtained by measuring the barycentric mean (BCM) fluorescence, while T_{agg} was determined as the temperature at which 50% increase in the static light scattering at a 266 nm wavelength relative to baseline was observed. Samples were concentrated to 1.0 mg/mL and subjected to a thermal ramp from 25 to 95° C. with 1° C. increments. The average and the standard error of three independent measurements were calculated using the UNit analysis software.

Stability was further analyzed under accelerated stress conditions. Multabodies diluted in 20 mM sodium phosphate pH 8.0, 150 mM NaCl, were concentrated to 10 mg/mL and incubated at 40° C. for four weeks. Each week, the percentage of properly folded protein was calculated based on the soluble content from SEC. A sample before (week 0) and after (week 4) the incubation period was assessed in a PsV neutralization assay to compare the functional activity of the molecules.

Virus production and TZM-bl neutralization assays. A panel of 14 HIV-1 pseudotyped viruses was generated by co-transfection of 293T cells with the HIV-1 subtype B backbone NL4-3.Luc.R⁻E plasmid (AIDS Research and Reference Reagent Program (ARRRP)) and the plasmid encoding the full-length Env clone, as previously described (45). HIV isolates X2088.c09, ZM106.9 and 3817.v2.c59 were kindly provided by the Collaboration for AIDS Vaccine Discovery (CAVD), and pCNE8, 1632_S2_B10, THR04156.18, 278-50, ZM197M.PB7, SF162, t257-31, Du422.1 and BG505 from NIH ARRRP. Mutation T332N in the BG505 Env expression vector was introduced by site-directed mutagenesis using the KOD-Plus mutagenesis kit (Toyobo, Osaka, Japan). The extended 25 HIV-1 pseudotyped panel was generated by adding HIV isolates p1054.TC4.1499, 6535, ZM214M.PL15, AC10.29, p16845, P6244_13.B5.4576, pM246F_C1G, TRJ04551, QH0692 and pCAAN5342 obtained from NIH ARRRP. Neutralization was determined in a single-cycle neutralization assay using the standard TZM-bl neutralization assay (45). Briefly, IgGs and Multabodies were incubated with a 10-15% tissue culture infectious dose of pseudovirus for 1 h at 37° C. prior to a 44-72 h incubation with TZM-bl cells. Virus neutralization was monitored by adding Britelite plus reagent (PerkinElmer) to the cells and measuring luminescence in relative light units (RLUs) using a Synergy Neo2 Multi-Mode Assay Microplate Reader (Biotek Instruments). HIV-1 Env pseudoviruses in the extended multiclade panel of 118 PsVs were generated by transfection in 293T cells of Env expression plasmids with full-length, Env-defective HIV genome SG3dEnv. HIV-1 pseudoviruses were incubated with Multabodies (primary concentration of 10 μg/ml and titrated 6-fold seven times) for 1 h at 37° C. before TZM-bl cells were added. Luciferase expression was quantified 48 h after infection upon cell lysis and the addition of luciferin substrate (Promega). For the neutralization assays done with parental IgGs, historical data at the Center for Virology and Vaccine Research, Harvard Medical School was used (primary concentration of 50 μg/ml and titrated 5-fold seven times). A cutoff limit of 10 g/mL was used to determine antibody breadth.

Antibody-dependent phagocytosis. 5 μL of red fluorescent Neutravidin microspheres (Invitrogen, F8775) were washed twice with PBS+0.1% BSA and incubated with 10 μg of biotinylated 93TH057 antigen. Biotinylation was performed using the EZ-link Sulfo-NHS biotinylation kit (Thermo Scientific, 2143) following the manufacturer instructions. The final volume was brought to 200 μL with PBS/0.1% BSA and incubated overnight with rotation at 4° C. Beads were washed twice before use to remove unbound protein, and resuspended in 200 μL per 5 μL of unlabeled bead volume.

Immune complexes were formed by incubating 93TH057-coated fluorescent beads (10 μL per sample) with 10 μL of 1, 5 and 10 μg of Multabody or antibody preparations for 2 h at 37° C. THP-1 cells (ATCC TIB-202) were maintained at fewer than 5×10⁵ cells/mL in RMPI+10% FBS (Wisent); and added to immune complexes at a concentration of 5×10⁴ cells/well (in 200 μL) before incubation for 16 h at 37° C., 5% CO₂. After incubation, cells were pelleted and washed with PBS before staining with Live Dead Fixable Violet stain (Invitrogen, L34995) according to the manufacturer's protocol. Cells were washed with PBS and fixed with 2% paraformaldehyde for 20 min at RT. Fixed cells were pelleted and washed once with FACS buffer (PBS+10% FBS, 0.5 mM EDTA) and analyzed on a LSRII Flow Cytometer (BD Biosciences). Data was analyzed in FlowJo (BD Biosciences, Ashland, OR), and phagocytosis was quantified as a percentage increase in phagocytosis compared to 93TH057-coated beads in the absence of antibody. Anti-human FcR binding inhibitor antibody (Invitrogen, 14-9161-73) was added to the indicated samples at the recommended concentration as an additional control.

PBMC infection. Peripheral blood mononuclear cells (PBMCs) were obtained from three healthy blood donors, with all donors providing written informed consent. The study was approved by the University of Toronto's Research Ethics Board (protocol #00037384). Blood was collected in heparinized vacutainers (BD Biosciences) and PBMCs were subsequently isolated using density centrifugation with Lymphoprep (StemCell Technologies, Cat #07861). PBMCs were activated with phytohemagglutinin (PHA; Gibco) in the presence of recombinant human IL-2 (50 U/mL) in complete RPMI medium (Wisent), containing 10% fetal bovine serum (FBS, Wisent), streptomycin at 100 μg/mL and penicillin at 100 U/mL for 72 h prior to HIV-1 infection. After three days of activation, HIV-1 infection of cells was performed by addition of the CXCR4-tropic laboratory isolate IIIB (150 pg of p24 Gag antigen per well) to triplicate cultures of activated PBMCs in round-bottom 96-well plates seeded with 2×10⁵ cells per well in RPMI+10% FBS+25 U/mL of IL-2. Before overlaying the cells with virus, Multabodies (T-01 MB and T-01 MB.v2) or IgG cocktail were pre-incubated with virus for 1 h at RT. Infected cells were cultured in the presence/absence of Multabody or antibody controls at doses ranging from 0.01-10 ug/mL, as indicated. The levels of HIV-1 replication were assessed by measuring the extracellular release of p24 Gag protein in cell-free culture supernatants tested at day 7 post-infection using a high-sensitivity AlphaLISA p24 detection kit (PerkinElmer, Waltham, MA) on a BioTEK Synergy Plate Reader, according to the manufacturer's protocols.

Cell viability and flow cytometry. On day 7 of infection, cells were fixed in 2% PFA and harvested for viability testing via absolute counting by flow cytometry performed using a BD LSRFortessa (Becton Dickinson). Cell viability was determined by comparison of the total live-gated cell counts in Multabody- or antibody-treated wells to the number of cells recovered from untreated control wells. Cell viability data was analyzed using FACSDiva.

Pharmacokinetic studies. In vivo studies were performed using three 6-week-old female NOD/Shi-scid/IL-2Rγnull (NCG strain code 572, Charles River Laboratories) immunodeficient mice per group. Mice were hosted by groups of 4/6 individuals. Each mouse was uniquely identified. Animals were housed in a ventilated cage (type II (16×19×35 cm, floor area=500 cm²)) under the following controlled conditions: 22° C., 55% humidity and 12:12-hour light dark cycle 7 am: 7 pm. This study was reviewed and approved by the local ethic committee (CELEAG). T-01 MB composed of the scFab of antibodies PGDM1400, N49P7 and 10E8v4 and scFc fragments of IgG1 Fc containing i) no mutations and ii) the effector function silencing mutations L234A, L235A and P329G (LALAP) and the I253A mutation were used in the study. In addition, T-01 MB.v2 composed of the same antibody specificities with i) no Fc mutations and ii) with the half-life extension mutations (M428L/N434S) in the IgG1 Fc was included. Mice received a single subcutaneous injection of 5 mg/kg of Multabodies or the control samples (an IgG mixture matching the Fab specificity of the Multabody) in 200 μL of PBS (pH 7.5). Blood samples were collected at multiple time points and serum samples were assessed for levels of circulating antibodies by ELISA. Briefly, 96-well Pierce Nickel Coated Plates (Thermo Fisher) were coated with 50 μL at 0.5 μg/ml of each of the His₆ₓ-tagged antigens recognized by the MB: BG5050 D368R SOSIP.664 trimer, gp120 subunit 93TH057 and MPER peptide, to determine circulating sample concentrations using reagent-specific standard curves for IgGs and Multabodies. HRP-Protein A (Invitrogen) was used as a secondary molecule and the chemiluminescence signal was quantified using the Epoch 2 Microplate spectrophotometer with the software Biotek Gen5 3.03.

### Results

Potency of HIV-1 bNAbs can be Enhanced with Avidity

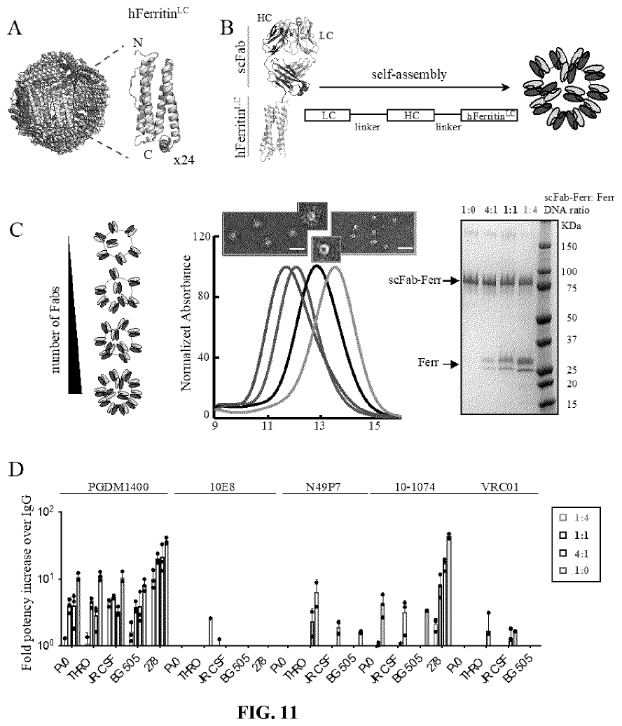

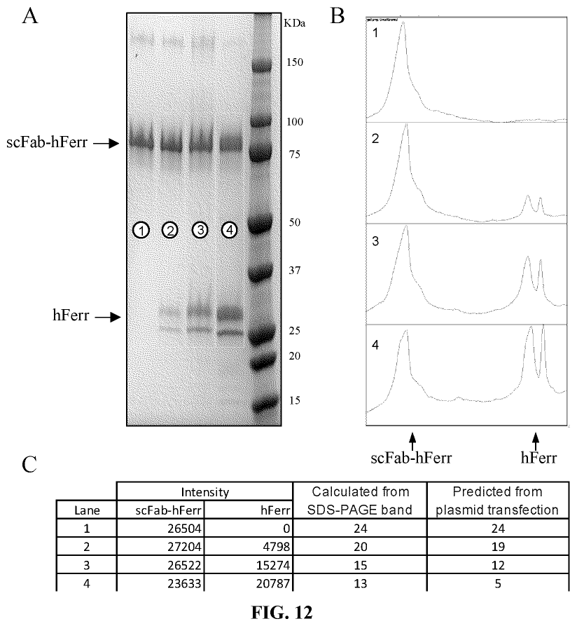

Apoferritin is a spherical nanocage of approximately 6 nm hydrodynamic radius formed by the self-oligomerization of 24 identical subunits (FIG. 11A). To investigate the impact of multi-valency on neutralization potency, we used the self-assembly properties of the light chain of human apoferritin to multimerize fragments of antigen binding (Fabs) derived from the most potent and broad HIV-1 bNAbs, which target different HIV-1 Env epitopes. Apoferritin subunits were genetically fused to single-chain Fabs (scFabs). scFabs were generated using flexible linkers between the light and heavy chains to ensure correct Fab heterodimerization. Apoferritin self-assembly drove multimerization of the scFab and displayed the antibody fragments at the nanocage periphery (FIG. 11B). Different densities of multimerized Fabs were achieved by co-transfection of scFab-human apoferritin-encoding plasmids together with different ratios of non-genetically modified human apoferritin (FIG. 11C, FIG. 12). The ability of the scFab-apoferritin fusions to block HIV-1 infection were compared to the corresponding IgGs using a small HIV-1 pseudovirus (PsV) panel (FIG. 11D). Strikingly, PGDM1400, one of the most potent anti-HIV bNAb described to date, showed 10- to 40-fold higher neutralization potency when multimerized via the light chain of apoferritin compared to its conventional IgG format. bNAb 10-1074 also showed a considerable improvement in neutralization potency (4- to 40-fold), whereas bNAbs 10E8, N49P7, and VRC01 showed no effect or more modest enhancements.

### Multabodies Potently and Broadly Neutralize HIV-1

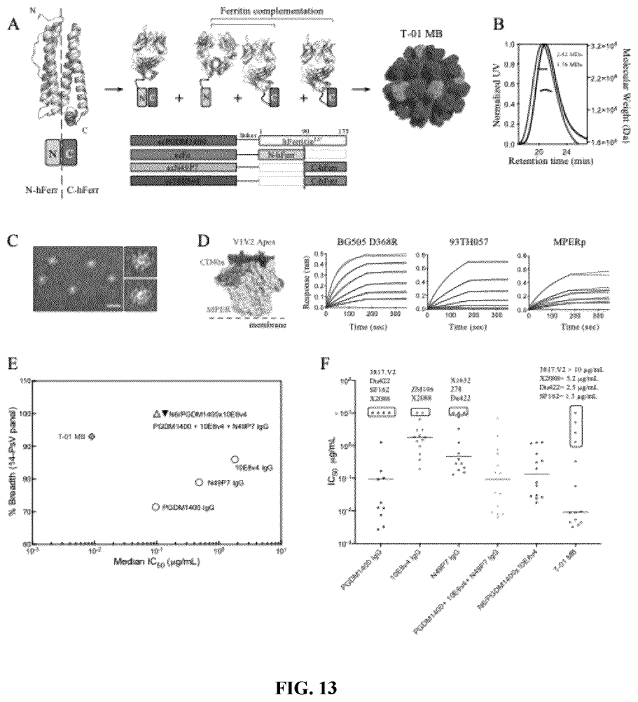

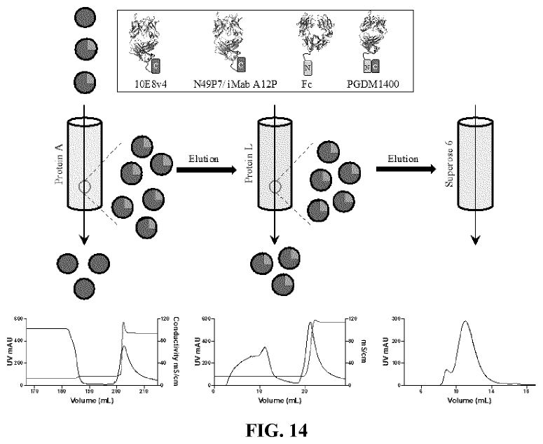

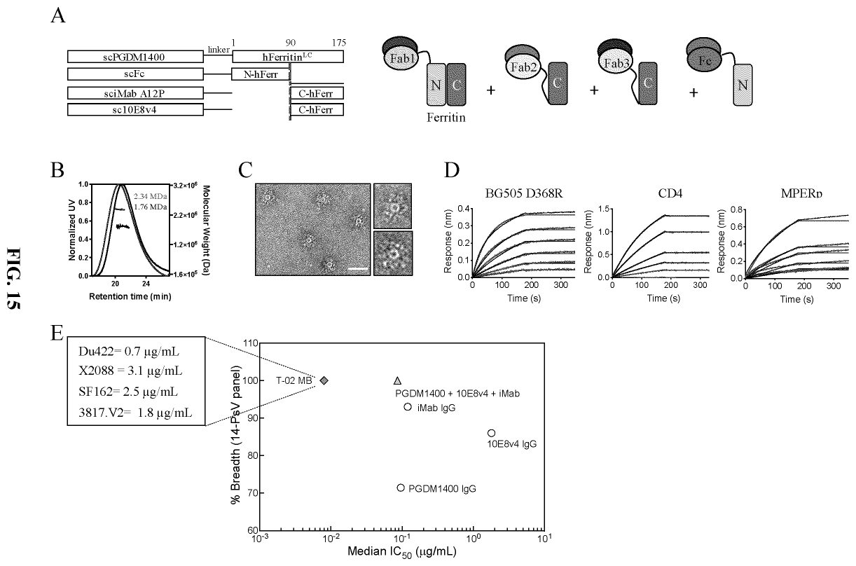

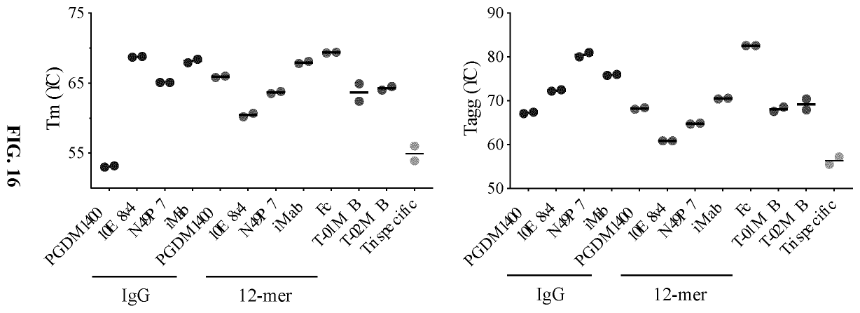

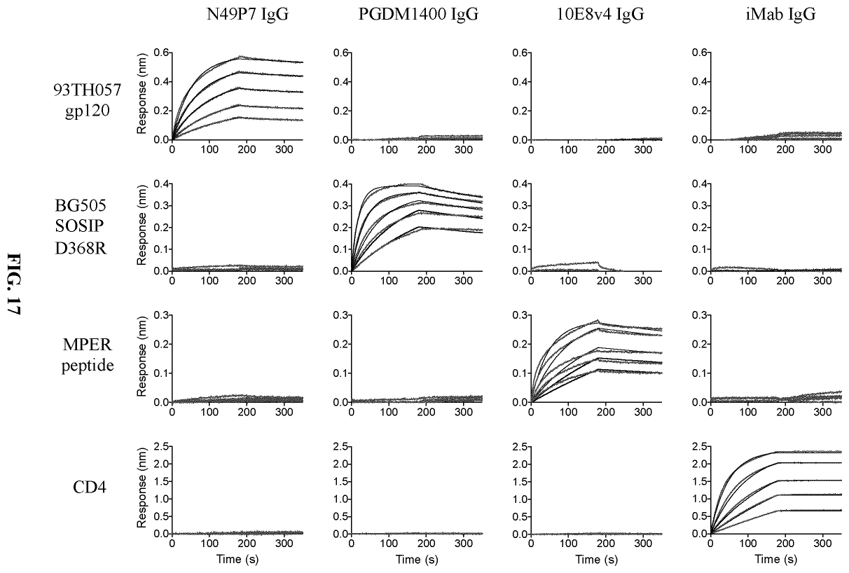

In view of these results, we sought to increase the coverage of PGDM1400 using our previously described Multabody platform based on an apoferritin split design (41). The strategy consists on the separation of the four-helix apoferritin subunit into two halves (N-ferritin and C-ferritin) and their N-terminal fusion to scFabs of different specificities (FIG. 13A). This approach allows inclusion of a higher number of Fabs on the surface of the nanocage resulting in a final molecule with higher avidity. In addition, the design allows the efficient combination of three different antibody specificities as well as a fragment crystallizable (Fc) to endow the molecule with IgG-like properties, such as ease of purification leveraging Protein A affinity (FIG. 14). Specifically, we combined scFab PGDM1400 with scFabs of the near-pan neutralizing antibodies 10E8v4 (a modified 10E8 with improved solubility (42)) and N49P7, and the single-chain construct of the Fc (scFc) of human IgG1 isotype (FIG. 13A). To explore whether a Multabody could also be designed that cross-targets the HIV-1 Env and its primary receptor, CD4, we replaced N49P7 with Ibalizumab (iMab), a CD4-directed post-attachment inhibitor that has been shown to effectively inhibit HIV-1 entry (43, 44) (FIG. 15A). The resulting tri-specific Multabodies, termed T-01 MB and T-02 MB, respectively, formed highly-decorated and homogeneous particles of around 2.4 MDa (FIG. 13B-C, FIG. 15B-C) with similar thermostability as the corresponding IgGs (FIG. 16). Epitope engagement by the tri-specific Multabodies was assessed in binding kinetics experiments using epitope-specific molecules: BG505 SOSIP D368R (PGDM1400), 93TH057 gp120/CD4 (N49P7/iMab), and a MPER peptide (10E8v4) (FIG. 17). Binding to the three epitope-specific antigens with high apparent binding affinities and no detectable dissociation confirms the presence of the three antibody specificities in the Multabodies (FIG. 13d, FIG. 15d).

Neutralization potency and breadth of the Multabodies was first assessed against a panel of 14 PsVs in a standardized in vitro TZM-bl neutralization assay (45). The 14-PsV panel was designed to include low-sensitivity PsVs with at least one PsV resistant to each bNAb being evaluated (cutoff IC₅₀ set at 10 μg/mL). The IC₅₀ value and breadth of the Multabodies were compared to (i) each individual IgG, (ii) an IgG cocktail that contains the same relative amount of each IgG present in the Multabody and (iii) the N6/PGDM1400×10E8v4 tri-specific antibody (46). T-01 MB and T-02 MB displayed 93% and 100% breadth (cutoff IC₅₀ set at 10 μg/mL) against this panel with a median IC₅₀ value of 0.009 μg/mL (3.9 pM) and 0.008 μg/mL (3.5 pM), respectively (FIG. 13E, FIG. 15E and Table 8). As such, there was a decrease of approximately one and two orders of magnitude in the median IC₅₀ values when calculated in g/mL and nM for the Multabodies compared to the IC₅₀ values of the IgG cocktails and the tri-specific antibody, respectively. Inspection of individual IC₅₀ values revealed that PsVs that are resistant to PGDM1400 IgG neutralization were also less sensitive to the Multabodies (FIG. 13F, FIG. 15E and Table 8). These data suggested that the neutralization property of the Multabodies is heavily dependent on one out of the three antibody specificities within the particle, in this case PGDM1400.

TABLE 8
IC₅₀ of individual and IgG mixtures, PGDM1400/N6x10E8v4 tri-specific and HIV-1 Multabodies against
a 14-PsV panel and 25-PsV panel (additional 11 HIV-1 strains highly resistant to PGDM1400).

| | Neutralization IC₅₀ μg/mL | | | | | | Neutralization IC₅₀ μg/mL | | | |
|---|---|---|---|---|---|---|---|---|---|---|
| | PGDM1400 | 10E8v4 | N49P7 | IgG | T-01 | T-01 | | iMab | IgG | |
| Virus ID | IgG | IgG | IgG | mix (T-01) | MB | MB.v2 | Tri-specific | IgG | mix (T-02) | T-02 MB |
| CRF02_AG | 0.012 | 1.5 | 1.2 | 0.021 | 0.0037 | 0.0016 | 0.028 | 0.095 | 0.0078 | 0.0015 |
| (Clone 257) | | | | | | | | | | |
| CRF02_AG | 0.16 | 1.8 | >10 | 0.16 | 0.0090 | 0.0016 | 1.2 | 0.67 | 0.14 | 0.0081 |
| (Clone 278) | | | | | | | | | | |
| X1632 | 0.018 | 2.0 | >10 | 0.042 | 0.0045 | 0.0029 | 0.030 | >10 | 0.036 | 0.0080 |
| ZM106.9 | 0.013 | >10 | 0.18 | 0.016 | 0.0089 | 0.0011 | 0.017 | 0.20 | 0.014 | 0.0031 |
| JRCSF | 0.0074 | 1.8 | 0.13 | 0.008 | 0.0054 | 0.0057 | 0.026 | 0.064 | 0.014 | 0.0040 |
| pTHRO4156 | 0.092 | 0.37 | 7.5 | 0.13 | 0.058 | 0.011 | 0.31 | 0.14 | 0.17 | 0.033 |
| 3817.V2 | >10 | 3.10 | 3.3 | 7.2 | >10 | 0.070 | 1.2 | 0.18 | 0.31 | 1.8 |
| PVO, clone 4 | 1.26 | 6.30 | 0.28 | 0.85 | 0.33 | 0.022 | 1.3 | 0.11 | 0.40 | 0.12 |
| X2088 | >10 | >10 | 0.38 | 1.5 | 5.2 | 0.11 | 0.56 | 0.13 | 0.66 | 3.1 |
| Du422 | >10 | 0.55 | >10 | 0.25 | 2.5 | 0.29 | 0.19 | 0.079 | 0.31 | 0.70 |
| ZM197M.PB7 | 0.10 | 0.19 | 0.58 | 0.031 | 0.0096 | 0.0049 | 0.073 | 0.042 | 0.062 | 0.0044 |
| pCNE8 | 0.0027 | 0.96 | 0.28 | 0.012 | 0.0032 | 0.0011 | 0.024 | 0.062 | 0.015 | 0.0013 |
| BG505 T332N | 0.0032 | 1.36 | 0.21 | 0.0088 | 0.0045 | 0.0016 | 0.059 | 0.12 | 0.013 | 0.0026 |
| SF162 | >10 | 3.03 | 0.15 | 0.56 | 1.3 | 0.39 | 0.33 | 7.5 | 1.4 | 2.5 |
| Median | 0.096 | 1.8 | 0.48 | 0.086 | 0.009 | 0.0049 | 0.13 | 0.12 | 0.10 | 0.008 |
| (14-PsV panel) | | | | | | | | | | |
| % Virus | 71 | 86 | 79 | 100 | 93 | 100 | 100 | 93 | 100 | 100 |
| Neutralized | | | | | | | | | | |
| p1054.TC4.1499 | >10 | 0.48 | 2.9 | 5.7 | 6.4 | 0.391 | μg/mL | | | |
| 6535, clone 3 | >10 | 0.25 | 0.85 | 5.4 | 1.9 | 0.06 | | | | |
| ZM214M.PL15 | >10 | 3.26 | 3.7 | >10 | 3.3 | 0.552 | | | | |
| AC10.29 | 3.05 | 0.8 | >10 | 0.15 | 0.0069 | 0.0032 | | | | |
| p16845 | >10 | 0.29 | 6.65 | 0.32 | 3.5 | 0.15 | | | | |
| p6244_{—} | >10 | 0.28 | 0.76 | 0.79 | 5.7 | 0.55 | | | | |
| 13.B5.4576 | | | | | | | | | | |
| pZM246F_C1G | >10 | 1.72 | 0.3 | 0.56 | 8.7 | 0.39 | | | | |
| TRJO4551 | >10 | 1.77 | 0.36 | 1.2 | 7.7 | 0.32 | | | | |
| QH0692 | >10 | 1.25 | 5.6 | 6 | >10 | 1.68 | | | | |
| NL4.3 | >10 | >10 | >10 | >10 | >10 | 1.8 | | | | |
| pCAAN5342.A2 | >10 | 3.31 | 8.5 | >10 | >10 | 0.74 | | | | |
| Median | 10 | 1.72 | 1.24 | 0.56 | 1.9 | 0.07 | | | | |
| (25-PsV panel) | | | | | | | | | | |
| % Virus | 44 | 88 | 80 | 88 | 84 | 100 | | | | |
| Neutralized | | | | | | | | | | |

### Engineering the Apoferritin Scaffold

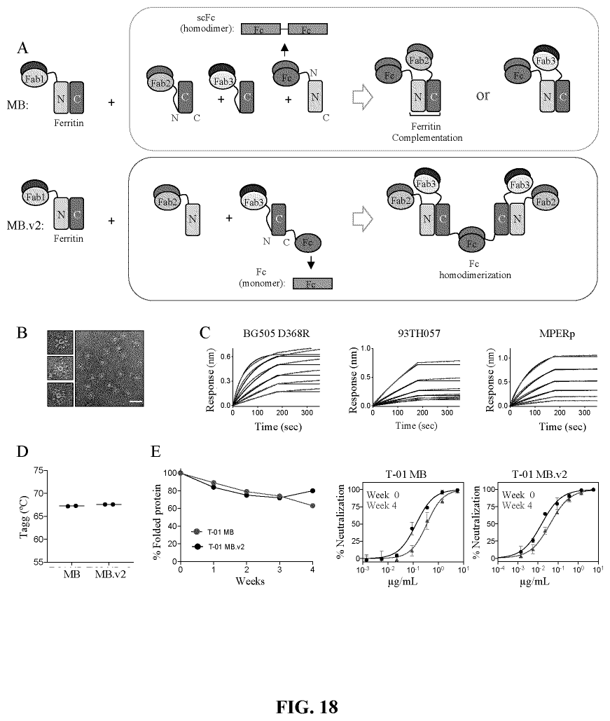

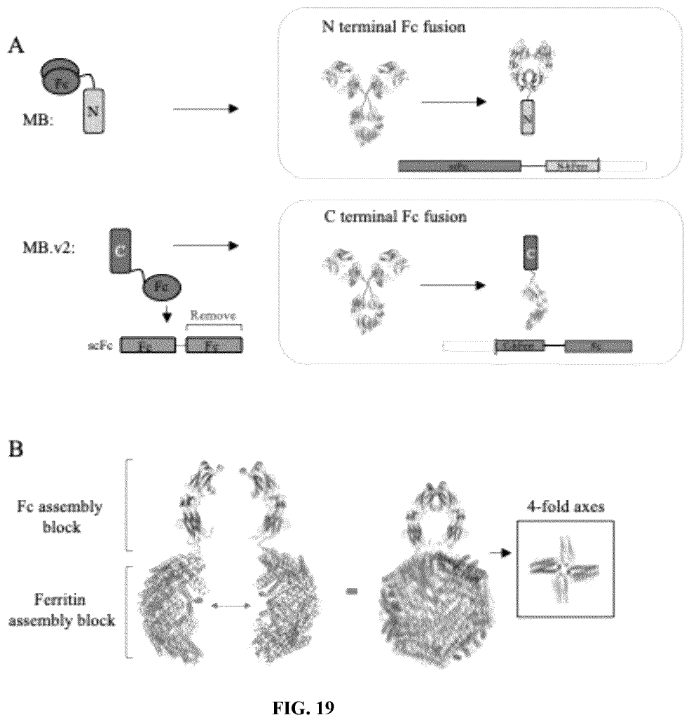

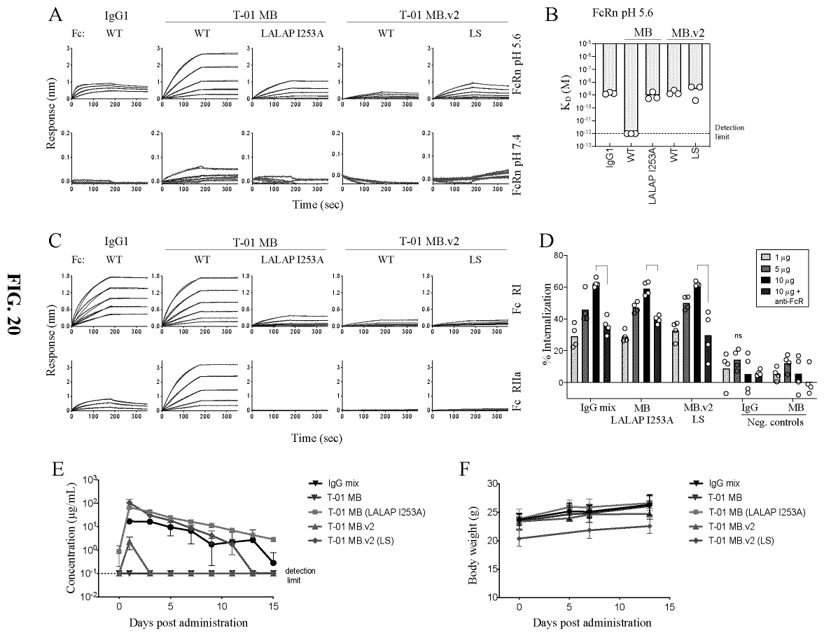

To further improve the neutralization properties of the Multabody, we introduced some modifications to its design and made a second-generation version (MB.v2). In the original MB, the scFc is located at the N terminus of the N-ferritin half, and only one Fab, either Fab2 or Fab3, is incorporated in the Multabody per each functional Fc homodimer (FIG. 18A, top). In comparison, the optimized MB.v2 contains a higher number of Fabs per Fc homodimer. To attain this, a monomeric Fc fragment (i.e. one Fc chain) and a scFab are positioned at the C terminus and the N terminus of the C-ferritin half, respectively (FIG. 18A bottom, FIG. 19A). As a result, dimerization of a functional Fc homodimer drives assembly of the MB.v2 particle together with split ferritin complementation and ferritin subunit oligomerization (FIG. 18A, FIG. 19B). Importantly, homodimerization to form one functional Fc ensures assembly of four Fabs different from PGDM1400 (i.e. two Fab2 and two Fab3), thus favoring a more balanced avidity for each of the three Fabs in the fully-assembled MB.v2.

The optimized Multabody design was tested in the T-01 background (PGDM1400, N49P7, 10E8v4) that targets three epitopes on HIV-1 Env. The resulting Multabody (T-01 MB.v2) assembled into well-formed spherical particles with no significant differences in morphology compared to the previously characterized T-01 MB (FIG. 18B). Antigen binding to BG505 SOSIP D368R, 93TH057 gp120, and MPER peptide confirmed correct folding of the three Fab specificities in T-01 MB.v2 (FIG. 18C). In addition, the new Multabody version preserves the same high thermal stability reported for T-01 MB, with a T_{agg} value of 67° C. (FIG. 18D). Multabodies were concentrated to 10 mg/mL and subjected to an accelerated stability test by incubating them at 40° C. for four weeks. Assessment of the amount of soluble protein over time revealed that the Multabodies were highly stable under these conditions, with over 70% of the sample remaining soluble for 30 days. Stability was further confirmed by only a modest loss in neutralization potency observed for the Multabodies at week 4 in comparison to their potency at week 0 (FIG. 18E).

### Pharmacokinetics of Multabodies is Similar to Corresponding IgGs

The antibody Fc domain has the capacity to interact with a variety of receptors, including Fc gamma receptors (FcγR's) and the neonatal Fc receptor (FcRn) conferring effector functions and in vivo half-life, respectively. However, Fc avidity can negatively impact the circulation time of molecules with multiple Fc fragments (41, 47). Indeed, T-01 MB showed strong binding to Fc receptors including to human FcRn at physiological pH (FIG. 20A-B), and high and low affinity FcγR's (FIG. 20C). Hence, we introduced the unique combination of LALAP (L234A, L235A and P329G) and I253A mutations in the Fc of T-01 MB to decrease binding to FcγR and FcRn, respectively, and achieve comparable binding observed for an IgG1 molecule (FIG. 20A-C). T-01 MB.v2 showed a more similar binding profile to IgG1, with comparable binding to human FcRn at acidic pH and no binding at physiological pH, even in the case of the half-life extension mutations LS (M428L/N434S) (FIG. 20A-B). Binding of T-01 MB.v2 to FcγR's yielded a low binding profile similar to that obtained with the LALAP FcR-silencing mutations in T-01 MB (FIG. 20C). The different binding patterns observed for the two MB versions are likely due to the different arrangement of the Fc fragments within the molecules (FIG. 18A, FIG. 19A). Despite low FcγRI binding, phagocytosis experiments using antigen-coated beads showed that both Multabody formats induced Fc-dependent internalization in THP-1 cells at levels similar to those achieved with the corresponding IgG mixture (FIG. 20D).

Next, we examined the in vivo bioavailability of both Multabody formats with and without engineered-Fc. A single dose of 5 mg/kg was administered subcutaneously in NOD/Shi-scid/IL-2Rγnull (NCG) immunodeficient mice and the amount of each molecule in the sera was measured every two days for 15 consecutive days. As expected from the in vitro characterization, only Fc-engineered Multabodies that had an IgG-like binding profile showed days of in vivo exposure with a similar rate of decay as the parental IgG cocktail (FIG. 20E). Multabody administration was well tolerated with no decrease in body weight (FIG. 20F) or visible adverse effects.

### Extraordinary Potency and Pan-Neutralization Breadth Achieved by MB.v2

We assessed the neutralization profile of T-01 MB.v2 against a PsV panel generated through addition of 11 HIV-1 strains highly resistant to PGDM1400 to our previous panel. The resulting 25-PsV panel contains 56% of PsV variants resistant (cutoff IC₅₀ set at 10 μg/mL) to PGDM1400 IgG neutralization (FIG. 21A-B). As expected, breadth and potency of the T-01 MB was greatly affected in the presence of PGDM1400 resistant PsVs (FIG. 21A-B, Table 8). However, as engineered, the neutralization profile of antibodies N49P7 and 10E8v4 were more dominant in T-01 MB.v2 allowing this optimized Multabody to achieve pan-neutralization while preserving the enhanced neutralization potency previously observed for this type of molecule (FIG. 21A-B, Table 8). When tested against an extended multiclade panel of 118 PsV, T-01 MB.v2 matched the pan-neutralization breadth of the corresponding IgG cocktail (100% virus coverage, cutoff IC₅₀ set at 10 μg/mL), yet displayed a remarkable neutralization potency (FIG. 21C-D, FIG. 22A and Table 9). Specifically, the IgG cocktail and T-01 MB were only able to neutralize 9% and 8% of the PsV with an IC₅₀ value of 0.001 μg/mL, respectively, while in the case of T-01 MB.v2, 50% of the PsVs were still neutralized with an IC₅₀ value of only 0.001 μg/mL (FIG. 21C). Remarkably, Multabodies achieved a median IC₅₀ value of only 0.0009 μg/mL (0.4 pM) and hence achieved pan-neutralization 32- and 490-fold more potently in mass and molarity, respectively, compared to the IgG cocktail (FIG. 21D). In addition, the IC₈₀ of T-01 MB.v2 confirmed its superior neutralization propensity over both the individual IgGs and the IgG cocktail, neutralizing 96% of all viral strains tested with a median IC₈₀ value of 0.005 μg/mL (2.2 pM) (FIG. 21C-D, FIG. 22A and Table 9). Importantly, Multabodies also blocked infection of primary peripheral blood mononuclear cells (PBMCs) with the replication-competent CXCR4-tropic HIV-1 IIIB strain (FIG. 22B), showing enhanced potency over the matched IgG mix, and without any impact on cell viability (FIG. 22C)

TABLE 9
Potency of parental and IgG mixtures and T-01 Multabody versions against a 118-PsV panel.

| | | PGDM1400 IgG | | 10E8 IgG | | N49P7 IgG | | IgG mix |
|---|---|---|---|---|---|---|---|---|
| Virus ID | Clade | IC₅₀ | IC₈₀ | IC₅₀ | IC₈₀ | IC₅₀ | IC₈₀ | IC₅₀ |
| 6535.3 | B | >10 | >10 | 0.081 | 1.32 | 0.186 | 0.598 | 0.526 |
| QH0692.42 | B | >10 | >10 | 0.442 | 4.046 | 0.663 | 2.411 | 1.296 |
| SC422661.8 | B | 0.436 | 6.530 | 0.445 | 2.715 | 0.114 | 0.320 | 0.160 |
| PVO.4 | B | 0.484 | 6.487 | 0.865 | 7.197 | 0.107 | 0.365 | 0.653 |
| TRO.11 | B | 0.283 | 1.376 | 0.034 | 0.250 | 0.044 | 0.160 | 0.092 |
| AC10.0.29 | B | 0.105 | 0.869 | 0.132 | 1.534 | 5.034 | >10 | 0.054 |
| RHPA4259.7 | B | 0.447 | 2.194 | 0.728 | 5.275 | 0.083 | 0.246 | 0.156 |
| THRO4156.18 | B | 0.072 | 0.348 | 0.156 | 0.884 | 2.341 | >10 | 0.032 |
| REJO4541.67 | B | 0.028 | 0.992 | 0.203 | 1.686 | 0.015 | 0.049 | 0.013 |
| TRJO4551.58 | B | >10 | >10 | 0.475 | 4.783 | 0.104 | 0.351 | 0.768 |
| WITO4160.33 | B | <0.001 | 0.008 | 0.094 | 1.226 | 0.084 | 0.248 | 0.002 |
| CAAN5342.A2 | B | >10 | >10 | 1.284 | 9.281 | 0.409 | 1.211 | 2.861 |
| WEAU_d15_{—} | B (T/F) | 0.103 | 1.366 | 6.039 | >10 | 0.045 | 0.117 | 0.050 |
| 410_787 | | | | | | | | |
| 1006_11_{—} | B (T/F) | 4.318 | >10 | 0.172 | 2.999 | 0.044 | 0.158 | 0.311 |
| C3_1601 | | | | | | | | |
| 1054_07_{—} | B (T/F) | >10 | >10 | 0.046 | 0.305 | 0.494 | 2.308 | 0.767 |
| TC4_1499 | | | | | | | | |
| 1056_10_{—} | B (T/F) | 3.258 | >10 | 0.134 | 1.028 | 0.141 | 0.468 | 0.191 |
| TA11_1826 | | | | | | | | |
| 1012_11_{—} | B (T/F) | 0.049 | 0.230 | 0.244 | 4.211 | 0.046 | 0.125 | 0.045 |
| TC21_3257 | | | | | | | | |
| 6240_08_{—} | B (T/F) | >10 | >10 | 0.756 | 5.591 | 0.457 | 1.728 | 1.673 |
| TA5_4622 | | | | | | | | |
| 6244_13_{—} | B (T/F) | >10 | >10 | 0.086 | 0.809 | 0.111 | 0.386 | 0.522 |
| B5_4576 | | | | | | | | |
| 62357_14_{—} | B (T/F) | >10 | >10 | 0.119 | 1.060 | 0.025 | 0.102 | 0.603 |
| D3_4589 | | | | | | | | |
| SC05_8C11_{—} | B (T/F) | 1.264 | 3.587 | 0.665 | 2.736 | 0.164 | 0.403 | 0.423 |
| 2344 | | | | | | | | |
| Du156.12 | C | 0.002 | 0.008 | 0.034 | 0.144 | 0.032 | 0.086 | <0.0006 |
| Du172.17 | C | 5.179 | >10 | 0.109 | 0.837 | 0.102 | 0.668 | 0.110 |
| Du422.1 | C | 0.694 | >10 | 0.351 | 1.883 | 4.496 | >10 | 0.375 |
| ZM197M.PB7 | C | 0.063 | 0.285 | 0.148 | 0.802 | 0.109 | 0.347 | 0.016 |
| ZM214M.PL15 | C | >10 | >10 | 0.896 | 5.021 | 0.213 | 0.953 | 0.941 |
| ZM233M.PB6 | C | <0.001 | 0.001 | 0.442 | 2.393 | 0.226 | 0.774 | <0.0006 |
| ZM249M.PL1 | C | 0.016 | 0.701 | 1.511 | 6.294 | 0.039 | 0.134 | 0.005 |
| ZM53M.PB12 | C | 0.002 | 0.011 | 3.248 | >10 | 0.251 | 0.800 | 0.001 |
| ZM109F.PB4 | C | 0.057 | 0.382 | 0.295 | 1.888 | 0.034 | 0.153 | 0.013 |
| ZM135M.PL | C | >10 | >10 | 0.171 | 1.186 | 0.200 | 0.960 | 0.227 |
| 10a | | | | | | | | |
| CAP45.2.00.G3 | C | <0.001 | 0.002 | 0.120 | 1.927 | 0.060 | 0.290 | 0.002 |
| CAP210.2.00.E8 | C | 0.034 | 0.514 | 0.798 | 3.892 | 1.817 | 6.485 | 0.057 |
| HIV-001428-2.42 | C | <0.001 | 0.027 | 0.809 | 7.298 | 0.006 | 0.020 | 0.006 |
| HIV-0013095-2.11 | C | 0.003 | 0.012 | 0.031 | 0.213 | 0.073 | 0.253 | 0.001 |
| HIV-16055-2.3 | C | <0.001 | 0.003 | 0.722 | 6.495 | 0.057 | 0.280 | 0.001 |
| HIV-16845-2.22 | C | >10 | >10 | 0.068 | 0.495 | 0.950 | 3.935 | 0.049 |
| Ce1086_B2 | C (T/F) | >10 | >10 | 1.162 | 6.934 | 0.089 | 0.568 | 0.442 |
| Ce0393_C3 | C (T/F) | 0.015 | 0.044 | 1.179 | 7.423 | 0.195 | 0.802 | 0.096 |
| Ce1176_A3 | C (T/F) | 0.587 | >10 | 0.867 | 3.923 | 0.204 | 0.589 | 0.102 |
| Ce2010_F5 | C (T/F) | >10 | >10 | 1.844 | 8.612 | 0.130 | 0.547 | 1.397 |
| Ce0682_E4 | C (T/F) | 0.010 | 0.043 | 1.215 | 7.709 | 0.041 | 0.131 | 0.012 |
| Ce1172_H1 | C (T/F) | 0.021 | 0.060 | 0.722 | 4.818 | 8.499 | >10 | 0.005 |
| Ce2060_G9 | C (T/F) | 0.001 | 0.007 | 1.019 | 5.030 | 0.661 | 2.547 | 0.002 |
| Ce703010054_2A2 | C (T/F) | 0.015 | 0.260 | 2.299 | 8.579 | 0.132 | 0.466 | 0.009 |
| BF1266.431a | C (T/F) | 0.003 | 0.011 | 1.070 | 8.418 | 0.057 | 0.199 | 0.005 |
| 246F C1G | C (T/F) | >10 | >10 | 2.217 | 9.937 | 0.094 | 0.255 | 0.815 |
| 249M B10 | C (T/F) | 0.039 | 0.786 | 1.271 | 9.050 | 0.081 | 0.377 | 0.008 |
| ZM247v1(Rev-) | C (T/F) | 0.019 | 0.051 | 0.506 | 6.745 | 0.062 | 0.214 | 0.015 |
| 7030102001E5(Rev-) | C (T/F) | >10 | >10 | 1.635 | >10 | 0.184 | 0.832 | 2.318 |
| 1394C9G1(Rev-) | C(T/F) | <0.001 | 0.004 | 0.963 | 5.327 | 3.841 | >10 | 0.001 |
| Ce704809221_1B3 | C (T/F) | 0.280 | 1.099 | 0.058 | 0.319 | 0.141 | 0.932 | 0.071 |
| CNE19 | BC | 0.001 | 0.007 | 0.666 | 6.485 | 0.009 | 0.041 | 0.001 |
| CNE20 | BC | 0.004 | 0.014 | 1.246 | 6.577 | 0.028 | 0.100 | 0.005 |
| CNE21 | BC | <0.001 | 0.002 | 0.410 | 3.181 | 0.756 | 6.020 | 0.001 |
| CNE17 | BC | 0.007 | 0.021 | 1.114 | 5.255 | 0.126 | 0.460 | 0.007 |
| CNE30 | BC | >10 | >10 | 1.560 | 9.842 | 0.180 | 0.609 | 1.135 |
| CNE52 | BC | 1.147 | 9.970 | 0.304 | 3.056 | 0.008 | 0.023 | 0.132 |
| CNE53 | BC | 0.077 | 0.246 | 0.326 | 1.916 | 0.035 | 0.124 | 0.027 |
| CNE58 | BC | 0.004 | 0.014 | 1.289 | 8.268 | 0.029 | 0.067 | <0.0006 |
| MS208.A1 | A | <0.001 | 0.139 | 0.609 | 3.717 | 0.120 | 0.479 | 0.004 |
| Q23.17 | A | <0.001 | 0.003 | 1.637 | 7.040 | 0.052 | 0.210 | <0.0006 |
| Q461.e2 | A | 0.089 | 0.572 | 0.786 | 3.638 | 0.261 | 0.922 | 0.041 |
| Q769.d22 | A | 0.002 | 0.018 | 1.667 | 10.000 | 0.025 | 0.111 | 0.001 |
| Q259.d2.17 | A | 0.166 | >10 | 3.956 | >10 | 0.332 | 2.474 | 0.890 |
| Q842.d12 | A | <0.001 | 0.002 | 1.824 | 7.270 | 0.021 | 0.060 | <0.0006 |
| 0260.v5.c36 | A | 0.033 | 0.175 | 6.452 | >10 | 0.354 | 0.884 | 0.011 |
| 3415.v1.cl | A | 0.036 | 0.327 | 2.481 | 12.306 | 0.160 | 0.550 | 0.068 |
| 3365.v2.c20 | A | 0.004 | 0.031 | 1.496 | 4.796 | 0.070 | 0.186 | 0.026 |
| 191955_A11 | A (T/F) | 0.001 | 0.021 | 0.680 | 3.532 | 1.811 | 8.870 | 0.001 |
| 191084 B7-19 | A (T/F) | <0.001 | 0.007 | 1.653 | 8.138 | 0.047 | 0.142 | <0.0006 |
| 9004SS_A3_4 | A (T/F) | <0.001 | 0.002 | 1.160 | 6.761 | 0.125 | 0.390 | <0.0006 |
| T257-31 | CRF02 AG | 0.002 | 0.031 | 0.880 | 5.779 | 0.158 | 0.619 | <0.0006 |
| 928-28 | CRF02_AG | 0.220 | 1.246 | 0.384 | 1.962 | 0.167 | 0.579 | 0.017 |
| 263-8 | CRF02_AG | 0.020 | 0.103 | 0.196 | 1.728 | 0.056 | 0.201 | 0.002 |
| T250-4 | CRF02_AG | <0.001 | <0.001 | 1.346 | 8.130 | 2.070 | >10 | <0.0006 |
| T251-18 | CRF02_AG | 8.615 | >10 | 0.310 | 1.993 | 0.178 | 0.620 | 0.376 |
| T278-50 | CRF02_AG | 0.574 | 4.942 | 0.949 | 7.460 | >10 | >10 | 0.049 |
| T255-34 | CRF02_AG | 0.654 | >10 | 0.778 | 4.207 | 0.053 | 0.262 | 0.078 |
| 211-9 | CRF02_AG | 0.088 | 0.393 | 1.091 | 6.508 | 0.220 | 0.767 | 0.024 |
| 235-47 | CRF02_AG | 0.005 | 0.032 | 0.384 | 2.072 | 0.045 | 0.300 | <0.0006 |
| 620345.c01 | CRF01_AE | 0.009 | >10 | 0.455 | 6.002 | >10 | >10 | 0.594 |
| CNE8 | CRF01_AE | <0.001 | 0.010 | 0.100 | 0.927 | 0.037 | 0.125 | 0.001 |
| C1080.c03 | CRF01_AE | <0.001 | <0.001 | 0.061 | 0.731 | 0.114 | 0.765 | <0.0006 |
| R2184.c04 | CRF01_AE | 0.003 | 0.012 | 0.272 | 2.455 | 0.011 | 0.031 | 0.007 |
| R1166.c01 | CRF01_AE | 1.431 | 6.921 | 0.274 | 2.614 | 0.092 | 0.313 | 0.144 |
| R3265.c06 | CRF01_AE | 0.013 | 0.041 | 2.534 | >10 | 0.048 | 0.282 | 0.025 |
| C2101.c01 | CRF01_AE | 0.006 | 0.021 | 1.921 | 8.260 | 0.060 | 0.340 | 0.011 |
| C3347.c11 | CRF01_AE | 0.011 | 0.051 | 0.016 | 0.167 | 0.024 | 0.067 | 0.012 |
| C4118.c09 | CRF01_AE | 0.002 | 0.005 | 1.622 | 9.064 | 0.043 | 0.147 | 0.003 |
| CNE5 | CRF01_AE | <0.001 | <0.001 | 1.408 | 9.637 | 0.157 | 0.513 | 0.001 |
| BJOX009000.02.4 | CRF01_AE | 0.443 | 1.287 | 1.570 | 9.954 | 0.113 | 0.506 | 0.108 |
| BJOX015000.11.5 | CRF01_AE (T/F) | 0.520 | >10 | 0.051 | 1.909 | 0.036 | 0.157 | 0.345 |
| BJOX010000.06.2 | CRF01_AE (T/F) | 0.605 | 5.870 | 0.341 | 5.293 | 0.085 | 0.592 | 0.074 |
| BJOX025000.01.1 | CRF01_AE (T/F) | 0.087 | 0.573 | 0.107 | 6.325 | 0.038 | 0.236 | 0.053 |
| BJOX028000.10.3 | CRF01_AE (T/F) | 0.072 | 0.963 | 0.665 | 7.361 | 0.020 | 0.123 | 0.021 |
| X1193_cl | G | 0.219 | 0.781 | 0.497 | 4.195 | 0.016 | 0.060 | 0.113 |
| P0402_c2_11 | G | 0.010 | 0.033 | 0.172 | 3.288 | 0.014 | 0.061 | 0.013 |
| X1254_c3 | G | >10 | >10 | 2.338 | 8.836 | 2.969 | >10 | 6.824 |
| X2088_c9 | G | >10 | >10 | >10 | >10 | 0.116 | 0.338 | 2.221 |
| X2131_C1_B5 | G | 0.128 | 0.568 | 0.189 | 0.974 | 0.081 | 0.273 | 0.045 |
| P1981_C5_3 | G | 0.104 | 4.601 | 0.057 | 0.285 | 0.113 | 0.274 | 0.030 |
| X1632_S2_B10 | G | 0.055 | 1.445 | 1.976 | 9.684 | >10 | >10 | 0.028 |
| 3016.v5.c45 | D | >10 | >10 | 0.476 | 4.393 | 0.221 | 1.037 | 0.581 |
| A07412M1.vrc12 | D | 0.032 | 0.127 | 0.576 | 6.054 | 0.108 | 0.645 | 0.012 |
| 231965.c01 | D | 0.025 | 1.280 | 5.648 | >10 | 0.054 | 0.193 | 0.036 |
| 231966.c02 | D | 0.001 | 0.004 | 0.191 | 3.130 | 0.102 | 0.430 | 0.002 |
| 3817.v2.c59 | CD | >10 | >10 | 1.181 | 5.977 | 0.716 | 3.726 | 2.878 |
| 6480.v4.c25 | CD | >10 | >10 | 1.572 | >10 | 0.031 | 0.113 | 0.151 |
| 6952.v1.c20 | CD | 0.501 | >10 | 0.343 | 1.907 | >10 | >10 | 0.075 |
| 6811.v7.c18 | CD | >10 | >10 | 2.539 | >10 | 0.229 | 0.992 | 2.862 |
| 89-F1_2_25 | CD | 0.001 | 0.005 | 1.112 | 10.000 | >10 | >10 | 0.003 |
| 3301.v1.c24 | AC | 0.048 | 0.245 | 1.341 | 7.095 | 0.025 | 0.069 | 0.023 |
| 6041.v3.c23 | AC | 0.013 | 0.289 | 2.333 | 9.766 | 0.047 | 0.222 | 0.022 |
| 6540.v4.cl | AC | 0.004 | 0.028 | 1.899 | 10.000 | >10 | >10 | 0.008 |
| 6545.v4.c1 | AC | 0.003 | 0.039 | 1.778 | 8.229 | 2.001 | >10 | 0.011 |
| 0815.v3.c3 | ACD | >10 | >10 | 0.332 | 2.868 | 0.016 | 0.053 | 0.116 |
| 3103.v3.c10 | ACD | 0.671 | 1.998 | >10 | >10 | 0.158 | 0.509 | 0.220 |
| MuL V | Neg. | >10 | >10 | >10 | >10 | >10 | >10 | >10 |
| | Control | | | | | | | |
| Median | | 0.056 | 0.570 | 0.725 | 5.265 | 0.110 | 0.396 | 0.029 |
| % Virus Neutralized | | 81 | 71 | 98 | 91 | 95 | 88 | 100 |

| | | IgG mix | T-01 MB | | T-01 MB.v2 | |
|---|---|---|---|---|---|---|
| Virus ID | Clade | IC₈₀ | IC₅₀ | IC₈₀ | IC₅₀ | IC₈₀ |
| 6535.3 | B | 3.614 | 0.715 | >10 | 0.009 | 0.434 |
| QH0692.42 | B | 6.343 | >10 | >10 | 1.520 | 9.309 |
| SC422661.8 | B | 0.604 | 0.176 | 0.889 | 0.017 | 0.081 |
| PVO.4 | B | 2.329 | 0.157 | 1.151 | 0.017 | 0.112 |
| TRO.11 | B | 0.341 | 0.111 | 0.826 | 0.006 | 0.067 |
| AC10.0.29 | B | 0.273 | 0.019 | 0.108 | 0.001 | 0.010 |
| RHPA4259.7 | B | 0.731 | 0.053 | 0.372 | 0.010 | 0.067 |
| THRO4156.18 | B | 0.121 | 0.023 | 0.104 | 0.003 | 0.016 |
| REJO4541.67 | B | 0.130 | 0.001 | 0.006 | <0.00004 | 0.0005 |
| TRJO4551.58 | B | 3.517 | 8.675 | >10 | 0.298 | 2.489 |
| WITO4160.33 | B | 0.014 | 0.002 | 0.013 | 0.0001 | 0.001 |
| CAAN5342.A2 | B | >10 | >10 | >10 | 1.254 | 7.486 |
| WEAU_d15_{—} | B (T/F) | 0.811 | 0.001 | 0.007 | 0.0003 | 0.002 |
| 410_787 | | | | | | |
| 1006_11_{—} | B (T/F) | 1.498 | 0.013 | 0.059 | 0.001 | 0.006 |
| C3_1601 | | | | | | |
| 1054_07_{—} | B (T/F) | 3.645 | >10 | >10 | 1.843 | >10 |
| TC4_1499 | | | | | | |
| 1056_10_{—} | B (T/F) | 1.065 | 0.478 | 1.917 | 0.022 | 0.187 |
| TA11_1826 | | | | | | |
| 1012_11_{—} | B (T/F) | 0.184 | 0.023 | 0.156 | 0.001 | 0.015 |
| TC21_3257 | | | | | | |
| 6240_08_{—} | B (T/F) | 8.310 | 0.704 | 3.199 | 0.035 | 0.261 |
| TA5_4622 | | | | | | |
| 6244_13_{—} | B (T/F) | 2.654 | >10 | >10 | 0.605 | 4.641 |
| B5_4576 | | | | | | |
| 62357_14_{—} | B (T/F) | 2.220 | 5.734 | >10 | 0.005 | 0.032 |
| D3_4589 | | | | | | |
| SC05_8C11_{—} | B (T/F) | 1.544 | 2.278 | >10 | 0.011 | 0.054 |
| 2344 | | | | | | |
| Du156.12 | C | <0.0006 | 0.051 | 0.215 | 0.0004 | 0.001 |
| Du172.17 | C | 0.752 | 1.298 | 8.551 | 0.005 | 0.022 |
| Du422.1 | C | 3.455 | 6.972 | >10 | 0.021 | 1.216 |
| ZM197M.PB7 | C | 0.065 | 0.183 | 0.895 | 0.001 | 0.003 |
| ZM214M.PL15 | C | >10 | >10 | >10 | 2.352 | >10 |
| ZM233M.PB6 | C | 0.001 | 0.019 | 0.081 | 0.0005 | 0.001 |
| ZM249M.PL1 | C | 0.056 | 0.122 | 0.824 | 0.0006 | 0.002 |
| ZM53M.PB12 | C | 0.007 | 0.0004 | 0.002 | <0.00004 | 0.0003 |
| ZM109F.PB4 | C | 0.084 | 0.006 | 0.022 | 0.001 | 0.005 |
| ZM135M.PL | C | 1.080 | 2.467 | >10 | 0.091 | 1.290 |
| 10a | | | | | | |
| CAP45.2.00.G3 | C | 0.005 | 0.0004 | 0.002 | 0.0001 | 0.0007 |
| CAP210.2.00.E8 | C | 0.555 | 0.410 | 6.302 | 0.0008 | 0.005 |
| HIV-001428-2.42 | C | 0.050 | 0.001 | 0.003 | 0.0003 | 0.001 |
| HIV-0013095-2.11 | C | 0.005 | 0.001 | 0.004 | 0.0002 | 0.0008 |
| HIV-16055-2.3 | C | 0.004 | 0.0005 | 0.004 | 0.00004 | 0.001 |
| HIV-16845-2.22 | C | 0.272 | 9.848 | >10 | 0.385 | 3.286 |
| Ce1086_B2 | C (T/F) | 2.899 | 3.797 | >10 | 0.014 | 0.328 |
| Ce0393_C3 | C (T/F) | 0.614 | 0.118 | 2.008 | 0.0006 | 0.003 |
| Ce1176_A3 | C (T/F) | 0.574 | 0.065 | 0.442 | 0.0008 | 0.003 |
| Ce2010_F5 | C (T/F) | 5.243 | >10 | >10 | 0.957 | 9.262 |
| Ce0682_E4 | C (T/F) | 0.053 | 0.256 | 1.583 | 0.0008 | 0.004 |
| Ce1172_H1 | C (T/F) | 0.016 | 0.095 | 0.445 | 0.0003 | 0.002 |
| Ce2060_G9 | C (T/F) | 0.005 | 0.001 | 0.004 | <0.00004 | 0.0002 |
| Ce703010054_2A2 | C (T/F) | 0.060 | 0.003 | 0.011 | 0.0006 | 0.002 |
| BF1266.431a | C (T/F) | 0.013 | 0.0009 | 0.003 | 0.00005 | 0.0005 |
| 246F C1G | C (T/F) | 2.791 | 8.146 | >10 | 0.207 | 1.437 |
| 249M B10 | C (T/F) | 0.026 | 0.311 | 1.096 | 0.0002 | 0.0020 |
| ZM247v1(Rev-) | C (T/F) | 0.043 | 0.004 | 0.017 | 0.0006 | 0.004 |
| 7030102001E5(Rev-) | C (T/F) | >10 | >10 | >10 | 0.871 | >10 |
| 1394C9G1(Rev-) | C(T/F) | 0.008 | 0.042 | 0.448 | 0.0004 | 0.002 |
| Ce704809221_1B3 | C (T/F) | 0.242 | 0.014 | 0.104 | 0.002 | 0.010 |
| CNE19 | BC | 0.005 | 0.001 | 0.004 | 0.0002 | 0.001 |
| CNE20 | BC | 0.014 | 0.072 | 0.321 | 0.0004 | 0.002 |
| CNE21 | BC | 0.004 | 0.0004 | 0.003 | 0.0001 | 0.0007 |
| CNE17 | BC | 0.021 | 0.206 | 1.029 | 0.001 | 0.007 |
| CNE30 | BC | 4.984 | >10 | >10 | 1.315 | 6.682 |
| CNE52 | BC | 0.431 | 0.022 | 0.103 | 0.004 | 0.019 |
| CNE53 | BC | 0.097 | 0.008 | 0.028 | 0.001 | 0.005 |
| CNE58 | BC | 0.001 | 0.025 | 0.111 | 0.0002 | 0.001 |
| MS208.A1 | A | 0.116 | 0.507 | 3.255 | 0.001 | 0.007 |
| Q23.17 | A | 0.137 | 0.0003 | 0.002 | 0.0001 | 0.0005 |
| Q461.e2 | A | 0.217 | 0.011 | 0.052 | 0.0003 | 0.004 |
| Q769.d22 | A | 0.018 | 0.002 | 0.008 | 0.0001 | 0.001 |
| Q259.d2.17 | A | >10 | 0.006 | 0.310 | 0.00004 | 0.018 |
| Q842.d12 | A | 0.002 | 0.0006 | 0.002 | <0.00004 | 0.0002 |
| 0260.v5.c36 | A | 0.045 | 0.006 | 0.022 | 0.0006 | 0.003 |
| 3415.v1.cl | A | 0.352 | 0.006 | 0.028 | 0.0002 | 0.002 |
| 3365.v2.c20 | A | 0.131 | 0.004 | 0.021 | 0.0003 | 0.003 |
| 191955_A11 | A (T/F) | 0.006 | 0.151 | 1.213 | 0.0004 | 0.002 |
| 191084 B7-19 | A (T/F) | 0.004 | 0.0006 | 0.003 | <0.00004 | 0.0001 |
| 9004SS_A3_4 | A (T/F) | <0.0006 | 0.052 | 0.219 | 0.0001 | 0.0007 |
| T257-31 | CRF02 AG | 0.016 | 0.002 | 0.007 | 0.0001 | 0.0008 |
| 928-28 | CRF02_AG | 0.120 | 0.027 | 0.092 | 0.002 | 0.010 |
| 263-8 | CRF02_AG | 0.020 | 0.011 | 0.036 | 0.001 | 0.005 |
| T250-4 | CRF02_AG | <0.0006 | 0.0004 | 0.002 | 0.0001 | 0.0003 |
| T251-18 | CRF02_AG | 1.859 | 0.716 | 4.276 | 0.049 | 0.316 |
| T278-50 | CRF02_AG | 0.282 | 0.012 | 0.055 | 0.001 | 0.008 |
| T255-34 | CRF02_AG | 0.433 | 0.003 | 0.036 | 0.0003 | 0.004 |
| 211-9 | CRF02_AG | 0.097 | 0.016 | 0.074 | 0.002 | 0.006 |
| 235-47 | CRF02_AG | 0.005 | 0.004 | 0.014 | 0.0009 | 0.002 |
| 620345.c01 | CRF01_AE | >10 | 0.039 | >10 | 0.003 | 3.278 |
| CNE8 | CRF01_AE | 0.006 | 0.001 | 0.005 | 0.0002 | 0.001 |
| C1080.c03 | CRF01_AE | <0.0006 | 0.0007 | 0.002 | 0.0001 | 0.0003 |
| R2184.c04 | CRF01_AE | 0.020 | 0.004 | 0.012 | 0.0003 | 0.002 |
| R1166.c01 | CRF01_AE | 0.555 | 0.139 | 0.465 | 0.012 | 0.057 |
| R3265.c06 | CRF01_AE | 0.113 | 0.003 | 0.018 | 0.0008 | 0.004 |
| C2101.c01 | CRF01_AE | 0.036 | 0.005 | 0.031 | 0.0009 | 0.004 |
| C3347.c11 | CRF01_AE | 0.039 | 0.007 | 0.029 | 0.0006 | 0.003 |
| C4118.c09 | CRF01_AE | 0.007 | 0.002 | 0.006 | 0.0002 | 0.0007 |
| CNE5 | CRF01_AE | 0.003 | 0.001 | 0.004 | 0.0002 | 0.0007 |
| BJOX009000.02.4 | CRF01_AE | 0.432 | 0.039 | 0.236 | 0.005 | 0.023 |
| BJOX015000.11.5 | CRF01_AE (T/F) | 1.649 | 0.013 | 0.088 | 0.001 | 0.007 |
| BJOX010000.06.2 | CRF01_AE (T/F) | 1.262 | 0.022 | 0.153 | 0.001 | 0.007 |
| BJOX025000.01.1 | CRF01_AE (T/F) | 0.365 | 0.036 | 0.281 | 0.003 | 0.018 |
| BJOX028000.10.3 | CRF01_AE (T/F) | 0.151 | 0.014 | 0.174 | 0.0007 | 0.008 |
| X1193_cl | G | 0.490 | 0.045 | 0.218 | 0.002 | 0.015 |
| P0402_c2_11 | G | 0.035 | 0.006 | 0.025 | 0.0008 | 0.005 |
| X1254_c3 | G | >10 | 1.688 | >10 | 0.141 | 7.410 |
| X2088_c9 | G | 8.326 | 3.978 | >10 | 0.184 | 1.379 |
| X2131_C1_B5 | G | 0.164 | 0.021 | 0.098 | 0.002 | 0.014 |
| P1981_C5_3 | G | 0.137 | 0.010 | 0.044 | 0.001 | 0.004 |
| X1632_S2_B10 | G | 0.275 | 0.002 | 0.009 | <0.00004 | 0.0003 |
| 3016.v5.c45 | D | 5.079 | 4.892 | >10 | 0.099 | 2.159 |
| A07412M1.vrc12 | D | 0.040 | 0.003 | 0.017 | 0.0003 | 0.002 |
| 231965.c01 | D | 0.160 | 0.005 | 0.022 | 0.0009 | 0.006 |
| 231966.c02 | D | 0.005 | 0.003 | 0.009 | 0.0004 | 0.002 |
| 3817.v2.c59 | CD | >10 | 3.879 | >10 | 0.388 | >10 |
| 6480.v4.c25 | CD | 0.694 | 0.787 | 4.400 | 0.072 | 0.470 |
| 6952.v1.c20 | CD | 0.358 | 0.020 | 0.365 | 0.001 | 0.019 |
| 6811.v7.c18 | CD | >10 | >10 | >10 | 3.030 | >10 |
| 89-F1_2_25 | CD | 0.008 | 0.001 | 0.004 | 0.0001 | 0.0005 |
| 3301.v1.c24 | AC | 0.080 | 0.007 | 0.035 | 0.0008 | 0.008 |
| 6041.v3.c23 | AC | 0.148 | 0.004 | 0.014 | 0.0007 | 0.003 |
| 6540.v4.cl | AC | 0.081 | 0.004 | 0.014 | 0.0004 | 0.003 |
| 6545.v4.c1 | AC | 0.236 | 0.003 | 0.009 | 0.0002 | 0.0009 |
| 0815.v3.c3 | ACD | 0.520 | 5.031 | >10 | 0.311 | 2.426 |
| 3103.v3.c10 | ACD | 1.013 | 0.030 | 0.080 | 0.001 | 0.008 |
| MuL V | Neg. | >10 | >10 | >10 | >10 | >10 |
| | Control | | | | | |
| Median | | 0.155 | 0.022 | 0.106 | 0.0009 | 0.005 |
| % Virus Neutralized | | 93 | 92 | 80 | 100 | 96 |

### DISCUSSION

The recent AMP clinical trials have highlighted the anticipated importance of both potency and breadth of bNAbs to be viable therapeutics capable of protecting against HIV-1 infection. Leveraging the principle of antibody avidity used in our previously-described Multabody technology to improve the potency of antibodies against SARS-CoV-2 (41), here we have engineered a second-generation Multabody platform to deliver exceptional neutralization breadth and potency against the vast sequence diversity of HIV-1.

The most distinctive feature of the optimized Multabody design in comparison to the first generation Multabody format (41) is the relative number of Fabs that self-associate per functional Fc domain. In contrast to the 1:1 Fc: 10E8v4/N49P7 ratio imposed by the design of the previously described Multabody platform, in the design of T-01 MB.v2, two N49P7 Fabs and two 10E8v4 Fabs are incorporated into the MB per dimeric Fc. The higher number of these two Fabs in the optimized Multabody favors their avidity and, consequently, their greater contribution to the neutralization signature of the particle. This is in contrast to the T-01 MB, which primarily relies on the neutralization properties of PGDM1400. The more balanced contribution of each of the antibodies is reflected in the better functional properties of T-01 MB.v2, which displayed cross-clade neutralization coverage of 100% at a median IC₅₀ value of 0.0009 μg/mL. In addition, viral infection by 83% of the 118-pseudoviruses tested was blocked by T-01 MB.v2 with an IC₈₀ value below 1 μg/ml, which has been recently proposed as the potency threshold required to confer in vivo protection in humans (35). However, it remains unclear whether that predictor of protection should be considered in mass or molarity. Indeed, despite a similar hydrodynamic radius and geometrical size for the Multabody compared to an IgM (41), the Multabody is approximately 10× heavier in molar mass compared to an IgG. Therefore, if molarity is the relevant in vivo measure associated with protection, then T-01 MB.v2 exhibits an extraordinarily low median IC₅₀ value of 0.4 pM. Correspondingly, a potency of IC₈₀ below 6.7 nM (1 μg/ml molar equivalent for an IgG) was achieved in 96% of the 118-HIV-1 PsV strains. These remarkable neutralization properties surpass those obtained with previously described bispecific and tri-specific antibodies (46, 48-50). In these antibody formats, the limited avidity precludes combination of both high avidity and multi-specificity and, consequently, potency and breadth are restricted to that of the parental mAbs.

There is a growing trend in the field of biotherapeutics toward the development of molecules with high valency. Strategies range from the generation of dodeca-valency IgM-like molecules (51, 52) upon addition of the mu-tailpiece of IgM to the constant region of IgG, to the design of alternative antibody formats. Among them are fusions of Fabs in a linear head-to-tail manner (53), appended IgGs (54-56), or diabody combination in tandem (Tamdabs) (57) or fused to the CH3 of an IgG (di-diabody) (58). In addition, the use of multimerization scaffolds such as p53 (59), leucine zipper helixes (60), streptavidin (61), barnase-barstar modules (62), viral-like nanoparticles (63) and, more recently, de novo antibody cage-forming proteins (64), have been employed to overcome the limitation of IgG bivalency and improve the bioactivity of antibodies. Although attractive, these approaches face different challenges for their successful development as therapeutic agents. Multimeric antibody formats that rely on variable fragments (Fv) of antibodies are often associated with low stability and, consequently, a high propensity to aggregate (65). Furthermore, dissociation of non-covalent fusions dictated by the affinity constant of the complex can limit the in vivo long-term stability of the molecule. In sharp contrast, Multabodies build on full IgG components (Fab and Fc) that are fused to the thermostable, functionally-silent human apoferritin light chain scaffold, and thus are highly stable IgG-like molecules even under thermal stress. A mouse surrogate Multabody previously administered subcutaneously in immuno-competent C57BL/6 mice showed undetectable levels of anti-drug antibodies similarly to its parent IgG, providing proof-of-principle for the potentially low intrinsic immunogenicity of the Multabody platform (41). Future studies in higher organisms will help determine the immunogenicity of Multabodies encoded by human-derived sequences, which we propose might be dictated predominantly by the properties of the underlying antibody sequences.

Bioavailability of large biologics is an additional challenge associated with engineered approaches to increase avidity (63). Multabodies have been engineered to include Fc domains and hence enable FcRn-mediated recycling of the molecule. As a consequence of Fc avidity, binding of T-01 MB to FcRn at pH 6.0 was improved by orders of magnitude, however, the simultaneous affinity improvement at pH 7.4 limits the application of this strategy towards enhancing half-life, and several mutations were needed to decrease Fc binding to FcRn and FcγR in order to not surpass the binding affinity observed for IgGs. Such enhanced Fc receptor avidity was not observed in the case of T-01 MB.v2, where fusion of the Fc chains at the C-terminus of apoferritin leads to the formation of particles with inverted and more distantly located Fc domains and, consequently, reduced Fc avidity. Similar to a previous study with a mouse surrogate Multabody (41), Fc avidity modulation strategies successfully resulted in Multabody molecules with remarkably similar rate of decay over time as the parental IgG cocktail. In addition to the favorable pharmacokinetic profile, Multabodies in both formats that possess residual binding to FcγR induced Fc-mediated phagocytosis in vitro to levels similar to the parental IgG mixture, at least in a THP-1 system that co-expresses both FcγRI and FcγRIIa (66). Future experiments will be needed to fully characterize the capacity of the Multabodies to trigger immune effector functions and their in vivo implications.

From the limited number of antibody specificities we characterized in this study, we observed that antibodies targeting epitopes located at the apex of the HIV-1 Env trimer such as PGDM1400 seem to experience the greatest benefit to neutralization potency when formulated as Multabodies. This increase in potency was less apparent as the epitope was located closer to the viral membrane, as in the case of 10E8. The dependence on epitope location for potency enhancement could be further impacted by the low surface spike density (67), the arrangement of those sparse Env trimers on the HIV-1 surface (68) or by the accessibility of certain epitopes that may be more or less sterically occluded. In light of this, it will be interesting to explore how the potency of antibodies against viruses with higher surface densities and closely spaced spikes can be enhanced by the Multabody platform, and further determine the impact of epitope location on potency enhancement mediated by avidity.

Replacement of N49P7 with iMab—a CD4-directed post-attachment inhibitor—resulted in a functional Multabody with potent neutralization activity, demonstrating that cross-targeting of viral epitopes and cellular receptors can be attained with this type of particle. This data raises the interesting possibility that the Multabody technology could also be implemented in other fields that promote the binding of receptors across separate entities, such as cell-cell interactions in immunotherapy. Overall, our protein engineering study demonstrates the versatility of human apoferritin as a modular nanocage to build antibody avidity and multi-specificity jointly towards enhanced functionality.

### REFERENCES

1. A. J. Conley, et al., Neutralization of divergent human immunodeficiency virus type 1 variants and primary isolates by IAM-41-2F5, an anti-gp41 human monoclonal antibody. Proc. Natl. Acad. Sci. U.S.A 91, 3348-3352 (1994).
2. G. Stiegler, et al., A Potent Cross-Clade Neutralizing Human Monoclonal Antibody against a Novel Epitope on gp41 of Human Immunodeficiency Virus Type 1. AIDS Res. Hum. Retroviruses 17, 1757-65 (2001).
3. M. B. Zwick, et al., Broadly Neutralizing Antibodies Targeted to the Membrane-Proximal External Region of Human Immunodeficiency Virus Type 1 Glycoprotein gp41. J Virol. 75, 10892-905 (2001).
4. A. Buchacher, et al., Generation of Human Monoclonal Antibodies against HIV-1 Proteins; Electrofusion and Epstein-Barr Virus Transformation for Peripheral Blood Lymphocyte Immortalization. AIDS Res. Hum. Retroviruses 10, 359-69 (1994).
5. C. F. Barbas, et al., Recombinant human Fab fragments neutralize human type 1 immunodeficiency virus in vitro. Proc. Natl. Acad. Sci. U.S.A 89, 9339-9343 (1992).
6. D. R. Burton, et al., Efficient neutralization of primary isolates of HIV-1 by a recombinant human monoclonal antibody. Science 266, 1024-1027 (1994).
7. X. Wu, et al., Rational design of envelope identifies broadly neutralizing human monoclonal antibodies to HIV-1. Science 329, 856-861 (2010).
8. J. F. Scheid, et al., Broad diversity of neutralizing antibodies isolated from memory B cells in HIV-infected individuals. Nature 458, 636-640 (2009).
9. D. Sok, et al., Recombinant HIV envelope trimer selects for quaternary-dependent antibodies targeting the trimer apex. Proc. Natl. Acad. Sci. U.S.A 111, 17624-17629 (2014).
10. L. M. Walker, et al., Broad neutralization coverage of HIV by multiple highly potent antibodies. Nature 477, 466-470 (2011).
11. N. A. Doria-Rose, et al., Developmental pathway for potent V1V2-directed HIV-neutralizing antibodies. Nature 509, 55-62 (2014).
12. J. Huang, et al., Broad and potent neutralization of HIV-1 by a gp41-specific human antibody. Nature 491, 406-412 (2012).
13. L. M. Walker, et al., Broad and potent neutralizing antibodies from an African donor reveal anew HIV-1 vaccine target. Science 326, 285-289 (2009).
14. M. M. Sajadi, et al., Identification of Near-Pan-neutralizing Antibodies against HIV-1 by Deconvolution of Plasma Humoral Responses. Cell 173, 1783-1795.el4 (2018).
15. J. F. Scheid, et al., Sequence and Structural Convergence of Broad and Potent HIV Antibodies That Mimic CD4 Binding. Science 333, 1633-1637 (2011).
16. T. Schoofs, et al., Broad and Potent Neutralizing Antibodies Recognize the Silent Face of the HIV Envelope. Immunity 50, 1513-1529.e9 (2019).
17. J. Huang, et al., Identification of a CD4-Binding-Site Antibody to HIV that Evolved Near-Pan Neutralization Breadth. Immunity 45, 1108-1121 (2016).
18. R. Pejchal, et al., A potent and broad neutralizing antibody recognizes and penetrates the HIV glycan shield. Science 334, 1097-1103 (2011).
19. C. Blattner, et al., Structural delineation of a quaternary, cleavage-dependent epitope at the gp41-gp120 interface on intact HIV-1 env trimers. Immunity 40, 669-680 (2014).
20. H. Mouquet, et al., Complex-type N-glycan recognition by potent broadly neutralizing HIV antibodies. Proc. Natl. Acad. Sci. U.S.A 109, E3268-77 (2012).
21. T. W. Baba, et al., Human neutralizing monoclonal antibodies of the IgG1 subtype protect against mucosal simian-human immunodeficiency virus infection. Nat. Med. 6, 200-206 (2000).
22. A. J. Hessell, et al., Effective, low-titer antibody protection against low-dose repeated mucosal SHIV challenge in macaques. Nat. Med. 15, 951-954 (2009).
23. A. J. Hessell, et al., Broadly neutralizing human anti-HIV antibody 2G12 is effective in protection against mucosal SHIV challenge even at low serum neutralizing titers. PLoS Pathog. 5, e1000433 (2009).
24. R. Hofmann-Lehmann, et al., Postnatal pre- and postexposure passive immunization strategies: Protection of neonatal macaques against oral simian-human immunodeficiency virus challenge. J Med. Primatol. 31, 109-119 (2002).
25. R. Hofmann-Lehmann, et al., Postnatal passive immunization of neonatal macaques with a triple combination of human monoclonal antibodies against oral simian-human immunodeficiency virus challenge. J. Virol. 75, 7470-7480 (2001).
26. Y. U. Van Der Velden, et al., Short Communication: Protective Efficacy of Broadly Neutralizing Antibody PGDM1400 Against HIV-1 Challenge in Humanized Mice. AIDS Res. Hum. Retroviruses 34, 790-793 (2018).
27. F. Klein, et al., HIV therapy by a combination of broadly neutralizing antibodies in humanized mice. Nature 492, 118-122 (2012).
28. M. Deruaz, et al., Protection of humanized mice from repeated intravaginal HIV challenge by passive immunization: A model for studying the efficacy of neutralizing antibodies in vivo. J Infect. Dis. 214, 612-616 (2016).
29. J. A. Horwitz, et al., HIV-1 suppression and durable control by combining single broadly neutralizing antibodies and antiretroviral drugs in humanized mice. Proc. Natl. Acad. Sci. U.S.A 110, 16538-16543 (2013).
30. S. Mehandru, et al., Adjunctive Passive Immunotherapy in Human Immunodeficiency Virus Type 1-Infected Individuals Treated with Antiviral Therapy during Acute and Early Infection. J Virol. 81, 11016-11031 (2007).
31. M. Caskey, et al., Viraemia suppressed in HIV-1-infected humans by broadly neutralizing antibody 3BNC117. Nature 522, 487-491 (2015).
32. M. Caskey, et al., Antibody 10-1074 suppresses viremia in HIV-1-infected individuals. Nat. Med. 23, 185-191 (2017).
33. R. M. Lynch, et al., Virologic effects of broadly neutralizing antibody VRC01 administration during chronic HIV-1 infection. Sci. Transl. Med. 7, 319ra206 (2015).
34. J. E. Ledgerwood, et al., Safety, pharmacokinetics and neutralization of the broadly neutralizing HIV-1 human monoclonal antibody VRC01 in healthy adults. Clin. Exp. Immunol. 182, 289-301 (2015).
35. L. Corey, et al., Two Randomized Trials of Neutralizing Antibodies to Prevent HIV-1 Acquisition. N. Engl. J. Med. 384, 1003-1014 (2021).
36. R. S. Rudicell, et al., Enhanced Potency of a Broadly Neutralizing HIV-1 Antibody In Vitro Improves Protection against Lentiviral Infection In Vivo. J Virol. 88, 12669-12682 (2014).
37. Y. D. Kwon, et al., Surface-Matrix Screening Identifies Semi-specific Interactions that Improve Potency of a Near Pan-reactive HIV-1-Neutralizing Antibody. Cell Rep. 22, 1798-1809 (2018).
38. E. Rujas, et al., Functional Optimization of Broadly Neutralizing HIV-1 Antibody 10E8 by Promotion of Membrane Interactions. J. Virol. 92, e02249-17 (2018).
39. E. Rujas, et al., Affinity for the Interface Underpins Potency of Antibodies Operating In Membrane Environments. Cell Rep. 32, 108037 (2020).
40. R. Diskin, et al., Increasing the potency and breadth of an HIV antibody by using structure-based rational design. Science 334, 1289-1293 (2011).
41. E. Rujas, et al., Multivalency transforms SARS-CoV-2 antibodies into broad and ultrapotent neutralizers. Nat Commun 12, 3661 (2021).
42. Y. D. Kwon, et al., Optimization of the Solubility of HIV-1-Neutralizing Antibody 10E8 through Somatic Variation and Structure-Based Design. J. Virol. 90, 5899-5914 (2016).
43. J. M. Jacobson, et al., Safety, pharmacokinetics, and antiretroviral activity of multiple doses of ibalizumab (formerly TNX-355), an anti-CD4 monoclonal antibody, in human immunodeficiency virus type 1-infected adults. Antimicrob. Agents Chemother. 53, 450-457 (2009).
44. D. R. Kuritzkes, et al., Antiretroviral Activity of the Anti-CD4 Monoclonal Antibody TNX-355 in Patients Infected with HIV Type 1. J Infect. Dis. 189, 286-291 (2004).
45. D. C. Montefiori, Measuring HIV neutralization in a luciferase reporter gene assay. Methods Mol. Biol. 485, 395-405 (2009).
46. L. Xu, et al., Trispecific broadly neutralizing HIV antibodies mediate potent SHIV protection in macaques. Science 358, 85-90 (2017).
47. O. S. Qureshi, et al., Multivalent Fcγ-receptor engagement by a hexameric Fc-fusion protein triggers Fcγ-receptor internalisation and modulation of Fcγ-receptor functions. Sci. Rep. 7 (2017).
48. M. Asokan, et al., Bispecific Antibodies Targeting Different Epitopes on the HIV-1 Envelope Exhibit Broad and Potent Neutralization. J Virol. 89, 12501-12512 (2015).
49. S. N. Khan, et al., Targeting the HIV-1 Spike and Coreceptor with Bi- and Trispecific Antibodies for Single-Component Broad Inhibition of Entry. J Virol. 92, e00384-18 (2018).
50. J. J. Steinhardt, et al., Rational design of a trispecific antibody targeting the HIV-1 Env with elevated anti-viral activity. Nat. Commun. 9 (2018).
51. R. I. Smith, M. J. Coloma, S. L. Morrison, Addition of a mu-tailpiece to IgG results in polymeric antibodies with enhanced effector functions including complement-mediated cytolysis by IgG4. J. Immunol. 154, 2226-36 (1995).
52. T. Olafsen, B. I. Rasmussen, L. Norderhaug, Ø. S. Bruland, I. Sandlie, IgM secretory tailpiece drives multimerisation of bivalent scFv fragments in eukaryotic cells. Immunotechnology 4, 141-153 (1998).
53. K. Miller, et al., Design, Construction, and In Vitro Analyses of Multivalent Antibodies. J. Immunol. 170, 4854-4861 (2003).
54. C. Wu, et al., Simultaneous targeting of multiple disease mediators by a dual-variable-domain immunoglobulin. Nat. Biotechnol. 25, 1290-1297 (2007).
55. C. Klein, W. Schaefer, J. T. Regula, The use of CrossMAb technology for the generation of bi- and multispecific antibodies. MAbs 8, 1010-1020 (2016).
56. A. Steinmetz, et al., CODV-Ig, a universal bispecific tetravalent and multifunctional immunoglobulin format for medical applications. MAbs 8, 867-878 (2016).
57. S. M. Kipriyanov, et al., Bispecific tandem diabody for tumor therapy with improved antigen binding and pharmacokinetics. J. Mol. Biol. 293, 41-56 (1999).
58. D. Lu, et al., Di-diabody: A novel tetravalent bispecific antibody molecule by design. J. Immunol. Methods 279, 219-232 (2003).
59. S. Kubetzko, E. Balic, R. Waibel, U. Zangemeister-Wittke, A. Pluckthun, PEGylation and multimerization of the anti-p185HER-2 single chain Fv fragment 4D5: Effects on tumor targeting. J. Biol. Chem. 281, 35186-35201 (2006).
60. P. Pack, K. Müller, R. Zahn, A. Pluckthun, Tetravalent miniantibodies with high avidity assembling in Escherichia coli. J. Mol. Biol. 246, 28-34 (1995).
61. S. M. Kipriyanov, et al., Affinity enhancement of a recombinant antibody: Formation of complexes with multiple valency by a single-chain Fv fragment-core streptavidin fusion. Protein Eng. 9, 203-211 (1996).
62. S. M. Deyev, R. Waibel, E. N. Lebedenko, A. P. Schubiger, A. Pluckthun, Design of multivalent complexes using the barnase barstar module. Nat. Biotechnol. 21, 1486-1492 (2003).
63. M. A. G. Hoffmann, et al., Nanoparticles presenting clusters of CD4 expose a universal vulnerability of HIV-1 by mimicking target cells. Proc. Natl. Acad. Sci. U.S.A 117, 18719-18728 (2020).
64. R. Divine, et al., Designed proteins assemble antibodies into modular nanocages. Science 372, eabd99947 (2021).
65. A. Wörn, A. Pluckthun, Different equilibrium stability behavior of scFv fragments: Identification, classification, and improvement by protein engineering. Biochemistry 38, 8739-8750 (1999).
66. H. B. Fleit, C. D. Kobasiuk, The Human Cell Line THP-1 Expresses Fc-gamma-RI and Fc-gamma-RII. J Leukoc Biol 49, 556-565 (1991).
67. P. Zhu, et al., Electron tomography analysis of envelope glycoprotein trimers on HIV and simian immunodeficiency virus virions. Proc. Natl. Acad. Sci. U.S.A 100, 15812-15817 (2003).
68. J. Chojnacki, et al., Maturation-dependent HIV-1 surface protein redistribution revealed by fluorescence nanoscopy. Science 338, 524-528 (2012).
69. M. Graille, et al., Complex between Peptostreptococcus magnus protein L and a human antibody reveals structural convergence in the interaction modes of Fab binding proteins. Structure 9, 679-687 (2001).
70. T. Zhou, et al., Structural basis for broad and potent neutralization of HIV-1 by antibody VRC01. Science 329, 811-817 (2010).
71. E. O. Freed, D. J. Myers, R. Risser, Mutational analysis of the cleavage sequence of the human immunodeficiency virus type 1 envelope glycoprotein precursor gp160. J. Virol. 63, 4670-4675 (1989).

### SEQUENCE LISTING

Underlining within sequences indicate linker sequences; bolding within sequences indicate ferritin or ferritin subunit sequences; boxed and bolded residues indicate residues that are mutated relative to a reference molecule, e.g. relative to an IgG1 Fc.

hFTL
SEQ ID NO:1
MSSQIRQNYSTDVEAAVNSLVNLYLQASYTYLSLGFYFDRDDVALEGVSHFFRELAEEKREG
YERLLKMQNQRGGRALFQDIKKPAEDEWGKTPDAMKAAMALEKKLNQALLDLHALGSARTDP
HLCDFLETHFLDEEVKLIKKMGDHLTNLHRLGGPEAGLGEYLFERLTLRHD
N hFTL
SEQ ID NO:2
MSSQIRQNYSTDVEAAVNSLVNLYLQASYTYLSLGFYFDRDDVALEGVSHFFRELAEEKREG
YERLLKMQNQRGGRALFQDIKKPAEDEW
C hFTL
SEQ ID NO:3
GKTPDAMKAAMALEKKLNQALLDLHALGSARTDPHLCDFLETHFLDEEVKLIKKMGDHLTNL
HRLGGPEAGLGEYLFERLTLRHD
C_hFTL R/K Variant
SEQ ID NO:4
GKTPDAMKAAMALEKKLNQALLDLHALGSARTDPHLCDFLETHFLDEEVKLIKKMGDHLTNL
HRLGGPEAGLGEYLFERLTLKHD
IgG1 Fc
SEQ ID NO:5
DKTHTCPPCPAPELLGGPSVFLFPPKPKDTLMISRTPEVTCVVVDVSHEDPEVKENWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
IgG1 scFc
SEQ ID NO:6
DKTHTCPPCPAPELLGGPSVFLFPPKPKDTLMISRTPEVTCVVVDVSHEDPEVKENWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGDKTHTCPPCPAPEL
LGGPSVFLFPPKPKDTLMISRTPEVTCVVVDVSHEDPEVKENWYVDGVEVHNAKTKPREEQY
NSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQG
NVFSCSVMHEALHNHYTQKSLSLSPGK
Mouse IgG2a Fc
SEQ ID NO:7
KPCPPCKCPAPNLLGGPSVFIFPPKIKDVLMISLSPIVTCVVVDVSEDDPDVQISWFVNNVE
VHTAQTQTHREDYNSTLRVVSALPIQHQDWMSGKEFKCKVNNKDLPAPIERTISKPKGSVRA
PQVYVLPPPEEEMTKKQVTLTCMVTDEMPEDI YVEWTNNGKTELNYKNTEPVLDSDGSYFMY
SKLRVEKKNWVERNSYSCSVVHEGLHNHHTTKSFSRTPGK
IgG2 scFc
SEQ ID NO:8
VECPPCPAPPVAGPSVFLFPPKPKDTLMISRTPEVTCVVVDVSHEDPEVQFNWYVDGVEVHN
AKTKPREEQFNSTFRVVSVLTVVHQDWLNGKEYKCKVSNKGLPAPIEKTISKTKGQPREPQV
YTLPPSREEMTKNQVSLTCLVKGFYPSDISVEWESNGQPENNYKTTPPMLDSDGSFFLYSKL
TVDKSRWQQGNVFSCSVMHEALHNHYTQKSLSLSPGKGGGGSGGGGSGGGGSGGGGSGGGGS
GGGGSGGGGSGGGGSGGGGSGGGGSGGGGSGGGGSGGGGSGGGGVECPPCPAPPVAGPSVFL
FPPKPKDTLMISRTPEVTCVVVDVSHEDPEVQFNWYVDGVEVHNAKTKPREEQFNSTFRVVS
VLTVVHQDWLNGKEYKCKVSNKGLPAPIEKTISKTKGQPREPQVYTLPPSREEMTKNQVSLT
CLVKGFYPSDISVEWESNGQPENNYKTTPPMLDSDGSFFLYSKLTVDKSRWQQGNVFSCSVM
HEALHNHYTQKSLSLSPGK
IgG4 Fc
SEQ ID NO:9
KYGPPCPSCPAPEFLGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
IgG4 scFc
SEQ ID NO:10
KYGPPCPSCPAPEFLGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGKGGGGSGGGGSGGGGSGGGGSG
GGGSGGGGSGGGGSGGGGSGGGGSGGGGSGGGGSGGGGSGGGGSGGGGKYGPPCPSCPAPEF
LGGPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRWQEG
NVFSCSVMHEALHNHYTQKSLSLSLGK
PGDM1400 IgG1 HC
SEQ ID NO:11
QAQLVQSGPEVRKPGTSVKVSCKAPGNTLKTYDLHWVRSVPGQGLOWMGWISHEGDKKVIVE
RFKAKVTIDWDRSTNTAYLQLSGLTSGDTAVYYCAKGSKHRLRDYALYDDDGALNWAVDVDY
LSNLEFWGQGTAVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKENWYVDGVEVHN
AKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
PGDM1400 LC
SEQ ID NO:12
DFVLTQSPHSLSVTPGESASISCKSSHSLIHGDRNNYLAWYVOKPGRSPOLLIYLASSRASG
VPDRESGSGSDKDFTLKISRVETEDVGTYYCMQGRESPWTFGQGTKVDIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSENRGEC
PGDM1400 HC (Fab)
SEQ ID NO:13
QAQLVQSGPEVRKPGTSVKVSCKAPGNTLKTYDLHWVRSVPGQGLOWMGWISHEGDKKVIVE
RFKAKVTIDWDRSTNTAYLQLSGLTSGDTAVYYCAKGSKHRLRDYALYDDDGALNWAVDVDY
LSNLEFWGQGTAVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEPKSC
PGDM1400 scFab
SEQ ID NO:14
DFVLTQSPHSLSVTPGESASISCKSSHSLIHGDRNNYLAWYVOKPGRSPOLLIYLASSRASG
VPDRFSGSGSDKDFTLKISRVETEDVGTYYCMQGRESPWTFGQGTKVDIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSENRGECGGGGSGGGGSGGGGSGGGGSGGGGSGGGG
SGGGGSGGGGSGGGGSGGGGSGGGGSGGGGSGGGGSGGGGSQAQLVQSGPEVRKPGTSVKVS
CKAPGNTLKTYDLHWVRSVPGQGLOWMGWISHEGDKKVIVERFKAKVTIDWDRSTNTAYLQL
SGLTSGDTAVYYCAKGSKHRLRDYALYDDDGALNWAVDVDYLSNLEFWGQGTAVTVSSASTK
GPSVFPLAPSSKSTSGGTAALGCLVKDYFPEPVTVSWNSGALTSGVHTFPAVLOSSGLYSLS
SVVTVPSSSLGTQTYICNVNHKPSNTKVDKKVEPKSC
N49P7 IgG1 HC
SEQ ID NO:15
ADLVQSGAVVKKPGDSVRISCEAQGYRFPDYIIHWIRRAPGQGPEWMGWMNPMGGQVNIPWK
FQGRVSMTRDTSIETAFLDLRGLKSDDTAVYYCVRDRSNGSGKRFESSNWFLDLWGRGTAVT
IQSASTKGPSVFPLAPSSKSTSGGTAALGCLVKDYFPEPVTVSWNSGALTSGVHTFPAVLOS
SGLYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKENWYVDGVEVHNAKTKPREEQYNSTY
RVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVES
CSVMHEALHNHYTQKSLSLSPGK
N49P7 LC
SEQ ID NO:16
QSALTQPRSVSASPGQSVTISCTGTHNLVSWCQHQPGRAPKLLIYDFNKRPSGVPDRESGSG
SGGTASLTITGLQDDDDAEYFCWAYEAFGGGTKLTVLGQPKAAPSVTLFPPSSEELQANKAT
LVCLISDFYPGAVTVAWKADSSPVKAGVETTTPSKOSNNKYAASSYLSLTPEQWKSHRSYSC
QVTHEGSTVEKTVAPTEC
N49P7 HC (Fab)
SEQ ID NO:17
ADLVQSGAVVKKPGDSVRISCEAQGYRFPDYIIHWIRRAPGQGPEWMGWMNPMGGQVNIPWK
FQGRVSMTRDTSIETAFLDLRGLKSDDTAVYYCVRDRSNGSGKRFESSNWFLDLWGRGTAVT
IQSASTKGPSVFPLAPSSKSTSGGTAALGCLVKDYFPEPVTVSWNSGALTSGVHTFPAVLOS
SGLYSLSSVVTVPSSSLGTQTYICNVNHKPSNTKVDKKVEPKSC
N49P7 scFab
SEQ ID NO:18
QSALTQPRSVSASPGQSVTISCTGTHNLVSWCQHQPGRAPKLLIYDENKRPSGVPDRESGSG
SGGTASLTITGLQDDDDAEYFCWAYEAFGGGTKLTVLGQPKAAPSVTLFPPSSEELQANKAT
LVCLISDFYPGAVTVAWKADSSPVKAGVETTTPSKQSNNKYAASSYLSLTPEQWKSHRSYSC
QVTHEGSTVEKTVAPTECGGGGSGGGGSGGGGSGGGGSGGGGSGGGGSGGGGSGGGGSGGGG
SGGGGSGGGGSGGGGSGGGGSGGGGSADLVQSGAVVKKPGDSVRISCEAQGYRFPDYIIHWI
RRAPGQGPEWMGWMNPMGGQVNIPWKFQGRVSMTRDTSIETAFLDLRGLKSDDTAVYYCVRD
RSNGSGKRFESSNWELDLWGRGTAVTIQSASTKGPSVFPLAPSSKSTSGGTAALGCLVKDYF
PEPVTVSWNSGALTSGVHTFPAVLOSSGLYSLSSVVTVPSSSLGTQTYICNVNHKPSNTKVD
KKVEPKSC
10E8v4 IgG1 HC
SEQ ID NO:19
EVRLVESGGGLVKPGGSLRLSCSASGFDFDNAWMTWVRQPPGKGLEWVGRITGPGEGWSVDY
AESVKGRFTISRDNTKNTLYLEMNNVRTEDTGYYFCARTGKYYDFWSGYPPGEEYFQDWGQG
TLVIVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKENWYVDGVEVHNAKTKPREEQY
NSTYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQG
NVFSCSVMHEALHNHYTQKSLSLSPGK
10E8v4 LC
SEQ ID NO:20
SELTQDPAVSVALKQTVTITCRGDSLRSHYASWYQKKPGQAPVLLFYGKNNRPSGIPDRESG
SASGNRASLTITGAQAEDEADYYCSSRDKSGSRLSVFGGGTKLTVLSQPKAAPSVTLFPPSS
EELQANKATLVCLISDFYPGAVTVAWKADSSPVKAGVETTTPSKOSNNKYAASSYLSLTPEQ
WKSHRSYSCQVTHEGSTVEKTVAPTECS
10E8v4 HC (Fab)
SEQ ID NO:21
EVRLVESGGGLVKPGGSLRLSCSASGFDEDNAWMTWVRQPPGKGLEWVGRITGPGEGWSVDY
AESVKGRFTISRDNTKNTLYLEMNNVRTEDTGYYFCARTGKYYDFWSGYPPGEEYFQDWGQG
TLVIVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSC
10E8v4 scFab
SEQ ID NO:22
SELTQDPAVSVALKQTVTITCRGDSLRSHYASWYQKKPGQAPVLLFYGKNNRPSGIPDRESG
SASGNRASLTITGAQAEDEADYYCSSRDKSGSRLSVEGGGTKLTVLSQPKAAPSVTLFPPSS
EELQANKATLVCLISDFYPGAVTVAWKADSSPVKAGVETTTPSKQSNNKYAASSYLSLTPEQ
WKSHRSYSCQVTHEGSTVEKTVAPTECGGGGSGGGGSGGGGSGGGGSGGGGSGGGGSGGGGS
GGGGSGGGGSGGGGSGGGGSGGGGSGGGGSGGGGSEVRLVESGGGLVKPGGSLRLSCSASGF
DFDNAWMTWVRQPPGKGLEWVGRITGPGEGWSVDYAESVKGRFTISRDNTKNTLYLEMNNVR
TEDTGYYFCARTGKYYDFWSGYPPGEEYFQDWGQGTLVIVSSASTKGPSVFPLAPSSKSTSG
GTAALGCLVKDYFPEPVTVSWNSGALTSGVHTFPAVLQSSGLYSLSSVVTVPSSSLGTQTYI
CNVNHKPSNTKVDKKVEPKSC
iMab IgG1 HC
SEQ ID NO:23
QVQLQQSGPEVVKPGASVKMSCKASGYTFTSYVIHWVROKPGQGLDWIGYINPYNDGTDYDE
KFKGKATLTSDTSTSTAYMELSSLRSEDTAVYYCAREKDNYATGAWFAYWGQGTLVTVSSAS
TKGPSVFPLAPCSRSTSESTAALGCLVKDYFPEPVTVSWNSGALTSGVHTFPAVLOSSGLYS
LSSVVTVPSSSLGTKTYTCNVDHKPSNTKVDKRVESKYCDKTHTCPPCPAPELLGGPSVELE
PPKPKDTLMISRTPEVTCVVVDVSHEDPEVKENWYVDGVEVHNAKTKPREEQYNSTYRVVSV
LTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMH
EALHNHYTQKSLSLSPGK
iMab LC
SEQ ID NO:24
DIVMTQSPDSLAVSLGERVTMNCKSSQSLLYSTNQKNYLAWYQQKPGQSPKLLIYWASTRES
GVPDRFSGSGSGTDFTLTISSVQAEDVAVYYCQQYYSYRTFGGGTKLEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSENRGEC
iMab HC (Fab)
SEQ ID NO:25
QVQLQQSGPEVVKPGASVKMSCKASGYTFTSYVIHWVRQKPGQGLDWIGYINPYNDGTDYDE
KFKGKATLTSDTSTSTAYMELSSLRSEDTAVYYCAREKDNYATGAWFAYWGQGTLVTVSSAS
TKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYC
iMab scFab
SEQ ID NO:26
DIVMTQSPDSLAVSLGERVTMNCKSSQSLLYSTNQKNYLAWYQQKPGOSPKLLIYWASTRES
GVPDRFSGSGSGTDFTLTISSVQAEDVAVYYCQQYYSYRTFGGGTKLEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSENRGECGGGGSGGGGSGGGGSGGGGSGGGGSGGGG
SGGGGSGGGGSGGGGSGGGGSGGGGSGGGGGGGGSGGGGSQVQLQQSGPEVVKPGASVKMS
CKASGYTFTSYVIHWVRQKPGQGLDWIGYINPYNDGTDYDEKFKGKATLTSDTSTSTAYMEL
SSLRSEDTAVYYCAREKDNYATGAWFAYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNV
NHKPSNTKVDKKVEPKSC
PGDM1400-hFTL
SEQ ID NO:27
DFVLTQSPHSLSVTPGESASISCKSSHSLIHGDRNNYLAWYVQKPGRSPOLLIYLASSRASG
VPDRFSGSGSDKDFTLKISRVETEDVGTYYCMQGRESPWTFGQGTKVDIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSENRGECGGGGSGGGGSGGGGSGGGGSGGGGSGGGG
SGGGGSGGGGSGGGGSGGGGSGGGGSGGGGSGGGGSGGGGSQAQLVQSGPEVRKPGTSVKVS
CKAPGNTLKTYDLHWVRSVPGQGLOWMGWISHEGDKKVIVERFKAKVTIDWDRSTNTAYLQL
SGLTSGDTAVYYCAKGSKHRLRDYALYDDDGALNWAVDVDYLSNLEFWGQGTAVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDGGGGSGGGGSGGGGSGGGGSGGGG
SGGSSQIRQNYSTDVEAAVNSLVNLYLQASYTYLSLGFYFDRDDVALEGVSHFFRELAEEKR
EGYERLLKMQNQRGGRALFQDIKKPAEDEWGKTPDAMKAAMALEKKLNQALLDLHALGSART
DPHLCDFLETHFLDEEVKLIKKMGDHLTNLHRLGGPEAGLGEYLFERLTLRHD
N49P7-C hFTL
SEQ ID NO:28
QSALTQPRSVSASPGQSVTISCTGTHNLVSWCQHOPGRAPKLLIYDENKRPSGVPDRESGSG
SGGTASLTITGLQDDDDAEYFCWAYEAFGGGTKLTVLGQPKAAPSVTLEPPSSEELQANKAT
LVCLISDFYPGAVTVAWKADSSPVKAGVETTTPSKQSNNKYAASSYLSLTPEQWKSHRSYSC
QVTHEGSTVEKTVAPTECGGGGSGGGGSGGGGSGGGGSGGGGSGGGGSGGGGSGGGGSGGGG
SGGGGSGGGGSGGGGSGGGGSGGGGSADLVQSGAVVKKPGDSVRISCEAQGYRFPDYIIHWI
RRAPGQGPEWMGWMNPMGGQVNIPWKFQGRVSMTRDTSIETAFLDLRGLKSDDTAVYYCVRD
RSNGSGKRFESSNWFLDLWGRGTAVTIQSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVD
KKVEPKSCGGGGSGGGGSGGGGSGGGGSGGGGSGGGKTPDAMKAAMALEKKLNQALLDLHAL
GSARTDPHLCDFLETHFLDEEVKLIKKMGDHLTNLHRLGGPEAGLGEYLFERLTLRHD
N49P7-N_hFTL
SEQ ID NO:29
QSALTQPRSVSASPGQSVTISCTGTHNLVSWCQHQPGRAPKLLIYDENKRPSGVPDRESGSG
SGGTASLTITGLQDDDDAEYFCWAYEAFGGGTKLTVLGQPKAAPSVTLEPPSSEELQANKAT
LVCLISDFYPGAVTVAWKADSSPVKAGVETTTPSKOSNNKYAASSYLSLTPEQWKSHRSYSC
QVTHEGSTVEKTVAPTECGGGGSGGGGSGGGGSGGGGSGGGGSGGGGSGGGGSGGGGSGGGG
SGGGGSGGGGSGGGGSGGGGSGGGGSADLVQSGAVVKKPGDSVRISCEAQGYRFPDYIIHWI
RRAPGQGPEWMGWMNPMGGQVNIPWKFQGRVSMTRDTSIETAFLDLRGLKSDDTAVYYCVRD
RSNGSGKRFESSNWFLDLWGRGTAVTIQSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVD
KKVEPKSCGGGGSGGGGSGGGGSGGGGSGGGGSGGSSQIRQNYSTDVEAAVNSLVNLYLQAS
YTYLSLGFYFDRDDVALEGVSHFFRELAEEKREGYERLLKMQNORGGRALFQDIKKPAEDEW
10E8v4-C_hFTL
SEQ ID NO:30
SELTQDPAVSVALKQTVTITCRGDSLRSHYASWYQKKPGQAPVLLFYGKNNRPSGIPDRESG
SASGNRASLTITGAQAEDEADYYCSSRDKSGSRLSVFGGGTKLTVLSQPKAAPSVTLFPPSS
EELQANKATLVCLISDFYPGAVTVAWKADSSPVKAGVETTTPSKOSNNKYAASSYLSLTPEQ
WKSHRSYSCQVTHEGSTVEKTVAPTECGGGGSGGGGSGGGGSGGGGSGGGGSGGGGSGGGGS
GGGGSGGGGSGGGGSGGGGSGGGGSGGGGSGGGGSEVRLVESGGGLVKPGGSLRLSCSASGF
DFDNAWMTWVRQPPGKGLEWVGRITGPGEGWSVDYAESVKGRFTISRDNTKNTLYLEMNNVR
TEDTGYYFCARTGKYYDFWSGYPPGEEYFQDWGQGTLVIVSSASTKGPSVFPLAPSSKSTSG
GTAALGCLVKDYFPEPVTVSWNSGALTSGVHTFPAVLQSSGLYSLSSVVTVPSSSLGTQTYI
CNVNHKPSNTKVDKKVEPKSCGGGGSGGGGSGGGGSGGGGSGGGGSGGGGKTPDAMKAAMAL
EKKLNQALLDLHALGSARTDPHLCDFLETHFLDEEVKLIKKMGDHLTNLHRLGGPEAGLGEY
LFERLTLRHD
iMab-C_hFTL
SEQ ID NO:31
DIVMTQSPDSLAVSLGERVTMNCKSSQSLLYSTNOKNYLAWYQQKPGQSPKLLIYWASTRES
GVPDRESGSGSGTDFTLTISSVQAEDVAVYYCQQYYSYRTFGGGTKLEIKRTVAAPSVEIFP
PSDEQLKSGTASVVCLLNNFYPREAKVQWKVDNALQSGNSQESVTEQDSKDSTYSLSSTLTL
SKADYEKHKVYACEVTHQGLSSPVTKSENRGECGGGGSGGGGSGGGGSGGGGSGGGGSGGGG
SGGGGSGGGGSGGGGSGGGGSGGGGSGGGGSGGGGSGGGGSQVQLQQSGPEVVKPGASVKMS
CKASGYTFTSYVIHWVRQKPGQGLDWIGYINPYNDGTDYDEKFKGKATLTSDTSTSTAYMEL
SSLRSEDTAVYYCAREKDNYATGAWFAYWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNV
NHKPSNTKVDKKVEPKSCGGGGSGGGGSGGGGSGGGGSGGGGSGGGGKTPDAMKAAMALEKK
LNQALLDLHALGSARTDPHLCDFLETHFLDEEVKLIKKMGDHLTNLHRLGGPEAGLGEYLFE
RLTLRHD
10E8v4-C_hFTL-IgG1_Fc
SEQ ID NO:32
SELTQDPAVSVALKQTVTITCRGDSLRSHYASWYQKKPGQAPVLLFYGKNNRPSGIPDRESG
SASGNRASLTITGAQAEDEADYYCSSRDKSGSRLSVFGGGTKLTVLSQPKAAPSVTLFPPSS
EELQANKATLVCLISDFYPGAVTVAWKADSSPVKAGVETTTPSKOSNNKYAASSYLSLTPEQ
WKSHRSYSCQVTHEGSTVEKTVAPTECGGGGSGGGGSGGGGSGGGGSGGGGSGGGGSGGGGS
GGGGSGGGGSGGGGSGGGGSGGGGSGGGGSGGGGSEVRLVESGGGLVKPGGSLRLSCSASGF
DFDNAWMTWVRQPPGKGLEWVGRITGPGEGWSVDYAESVKGRFTISRDNTKNTLYLEMNNVR
TEDTGYYFCARTGKYYDFWSGYPPGEEYFQDWGQGTLVIVSSASTKGPSVFPLAPSSKSTSG
GTAALGCLVKDYFPEPVTVSWNSGALTSGVHTFPAVLQSSGLYSLSSVVTVPSSSLGTQTYI
CNVNHKPSNTKVDKKVEPKSCGGGGSGGGGSGGGGSGGGGSGGGGSGGGGKTPDAMKAAMAL
EKKLNQALLDLHALGSARTDPHLCDFLETHFLDEEVKLIKKMGDHLTNLHRLGGPEAGLGEY
LFERLTLRHDGGSGGSGGSGGSGGGSGGSGGSGGSGDKTHTCPPCPAPELLGGPSVFLFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
10E8v4-C_hFTL-IgG1_LS
SEQ ID NO:33
SELTQDPAVSVALKQTVTITCRGDSLRSHYASWYQKKPGQAPVLLFYGKNNRPSGIPDRESG
SASGNRASLTITGAQAEDEADYYCSSRDKSGSRLSVFGGGTKLTVLSQPKAAPSVTLFPPSS
EELQANKATLVCLISDFYPGAVTVAWKADSSPVKAGVETTTPSKQSNNKYAASSYLSLTPEQ
WKSHRSYSCQVTHEGSTVEKTVAPTECGGGGSGGGGSGGGGSGGGGSGGGGSGGGGSGGGGS
GGGGSGGGGSGGGGSGGGGSGGGGSGGGGSGGGGSEVRLVESGGGLVKPGGSLRLSCSASGF
DFDNAWMTWVRQPPGKGLEWVGRITGPGEGWSVDYAESVKGRFTISRDNTKNTLYLEMNNVR
TEDTGYYFCARTGKYYDFWSGYPPGEEYFQDWGQGTLVIVSSASTKGPSVFPLAPSSKSTSG
GTAALGCLVKDYFPEPVTVSWNSGALTSGVHTFPAVLQSSGLYSLSSVVTVPSSSLGTQTYI
CNVNHKPSNTKVDKKVEPKSCGGGGSGGGGSGGGGSGGGGSGGGGSGGGGKTPDAMKAAMAL
EKKLNQALLDLHALGSARTDPHLCDFLETHFLDEEVKLIKKMGDHLTNLHRLGGPEAGLGEY
LFERLTLRHDGGSGGSGGSGGSGGGSGGSGGSGGSGDKTHTCPPCPAPELLGGPSVELFPPK
PKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVK

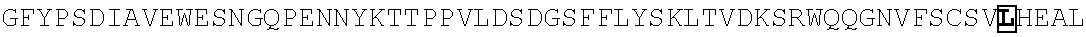

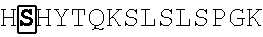

IgG1_scFc-N_hFTL
SEQ ID NO:34
DKTHTCPPCPAPELLGGPSVFLFPPKPKDTLMISRTPEVTCVVVDVSHEDPEVKENWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGDKTHTCPPCPAPEL
LGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGGGGSGGGGSGGSSQIRQNY
STDVEAAVNSLVNLYLQASYTYLSLGFYFDRDDVALEGVSHFFRELAEEKREGYERLLKMQN
QRGGRALFQDIKKPAEDEW
IgG2_scFc-N_hFTL
SEQ ID NO:35
VECPPCPAPPVAGPSVFLFPPKPKDTLMISRTPEVTCVVVDVSHEDPEVQFNWYVDGVEVHN
AKTKPREEQFNSTFRVVSVLTVVHQDWLNGKEYKCKVSNKGLPAPIEKTISKTKGQPREPQV
YTLPPSREEMTKNQVSLTCLVKGFYPSDISVEWESNGQPENNYKTTPPMLDSDGSFFLYSKL
TVDKSRWQQGNVFSCSVMHEALHNHYTQKSLSLSPGKGGGGSGGGGSGGGGSGGGGSGGGGS
GGGGSGGGGSGGGGSGGGGSGGGGSGGGGSGGGGSGGGGSGGGGVECPPCPAPPVAGPSVEL
FPPKPKDTLMISRTPEVTCVVVDVSHEDPEVQFNWYVDGVEVHNAKTKPREEQFNSTFRVVS
VLTVVHQDWLNGKEYKCKVSNKGLPAPIEKTISKTKGQPREPQVYTLPPSREEMTKNQVSLT
CLVKGFYPSDISVEWESNGQPENNYKTTPPMLDSDGSFFLYSKLTVDKSRWQQGNVFSCSVM
HEALHNHYTQKSLSLSPGKGGGGSGGGGSGGGGSGGGGSGGGGSGGSSQIRQNYSTDVEAAV
NSLVNLYLQASYTYLSLGFYFDRDDVALEGVSHFFRELAEEKREGYERLLKMQNORGGRALF
QDIKKPAEDEW
IgG4_scFc-N_hFTL
SEQ ID NO:36
KYGPPCPSCPAPEFLGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFEL
YSRLTVDKSRWQEGNVFSCSVMHEALHNHYTQKSLSLSLGKGGGGSGGGGSGGGGSGGGGSG
GGGSGGGGSGGGGSGGGGSGGGGSGGGGSGGGGSGGGGSGGGGSGGGGKYGPPCPSCPAPEF
LGGPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRWQEG
NVFSCSVMHEALHNHYTQKSLSLSLGKGGGGSGGGGSGGGGSGGGGSGGGGSGGSSQIRQNY
STDVEAAVNSLVNLYLQASYTYLSLGFYFDRDDVALEGVSHFFRELAEEKREGYERLLKMON
QRGGRALFQDIKKPAEDEW
IgG1_P329G scFc-N hFTL
SEQ ID NO:37
DKTHTCPPCPAPELLGGPSVFLFPPKPKDTLMISRTPEVTCVVVDVSHEDPEVKFNWYVDGV

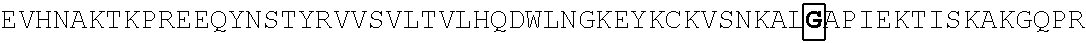

EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGDKTHTCPPCPAPEL
LGGPSVFLFPPKPKDTLMISRTPEVTCVVVDVSHEDPEVKENWYVDGVEVHNAKTKPREEQY

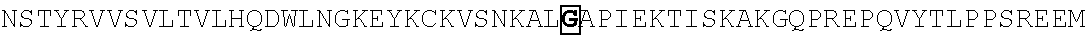

TKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGGGGSGGGGSGGSSQIRONY
STDVEAAVNSLVNLYLQASYTYLSLGFYFDRDDVALEGVSHFFRELAEEKREGYERLLKMON
QRGGRALFQDIKKPAEDEW
IgGI LALA scFc-N hFTL
SEQ ID NO:38

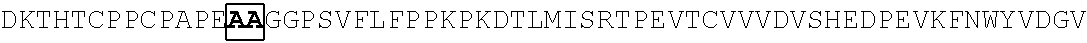

EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG

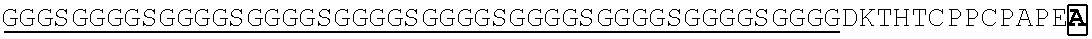

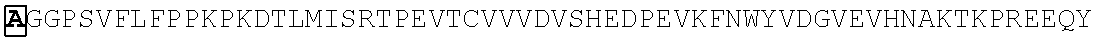

NSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGGGGSGGGGSGGSSQIRQNY
STDVEAAVNSLVNLYLQASYTYLSLGFYFDRDDVALEGVSHFFRELAEEKREGYERLLKMON
QRGGRALFQDIKKPAEDEW
IgG1_LALAP_scFc-N_hFTL
SEQ ID NO:39

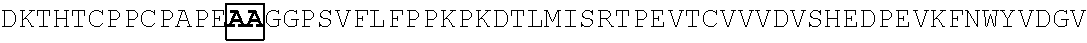

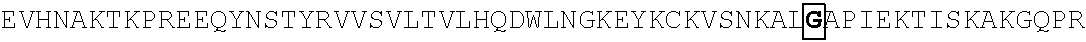

EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG

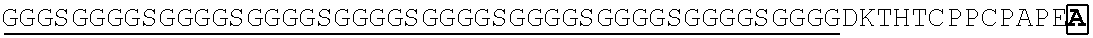

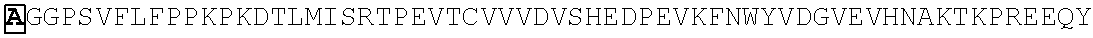

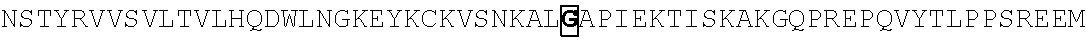

TKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGGGGSGGGGSGGSSQIRONY
STDVEAAVNSLVNLYLQASYTYLSLGFYFDRDDVALEGVSHFFRELAEEKREGYERLLKMON
QRGGRALFQDIKKPAEDEW
IgG1_K288A_scFc-N_hFTL
SEQ ID NO:40
DKTHTCPPCPAPELLGGPSVFLFPPKPKDTLMISRTPEVTCVVVDVSHEDPEVKENWYVDGV

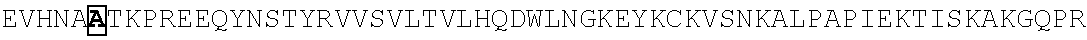

EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGDKTHTCPPCPAPEL

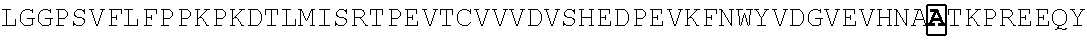

NSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGGGGSGGGGSGGSSQIRQNY
STDVEAAVNSLVNLYLQASYTYLSLGFYFDRDDVALEGVSHFFRELAEEKREGYERLLKMON
QRGGRALFQDIKKPAEDEW
IgG1_1253A_scFc-N_hFTL
SEQ ID NO:41

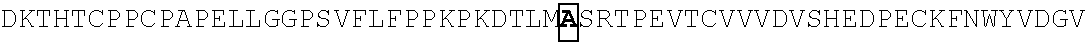

EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFEL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGDKTHTCPPCPAPEL

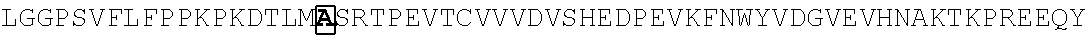

NSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGGGGSGGGGSGGSSQIRQNY
STDVEAAVNSLVNLYLQASYTYLSLGFYFDRDDVALEGVSHFFRELAEEKREGYERLLKMQN
QRGGRALFQDIKKPAEDEW
IgG1_1253V_scFc-N_hFTL
SEQ ID NO:42

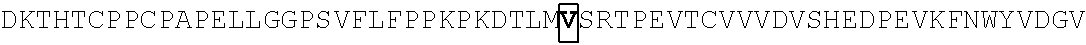

EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGSGGGGSGGGGSGGGGSGGGGSGGGGSGGGGDKTHTCPPCPAPEL

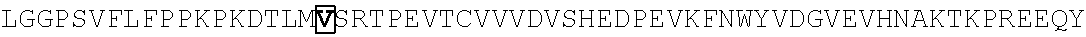

NSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGGGGSGGGGSGGSSQIRONY
STDVEAAVNSLVNLYLQASYTYLSLGFYFDRDDVALEGVSHFFRELAEEKREGYERLLKMQN
QRGGRALFQDIKKPAEDEW
IgG1_LALAP_1253A_scFc-N_hFTL
SEQ ID NO:43

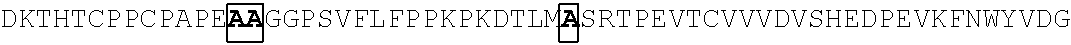

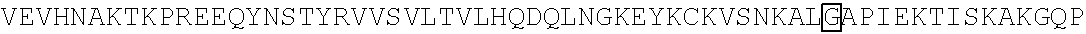

REPQVYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPGKGGGGSGGGGSGGGGSGGGGS
GGGGSGGGGSGGGGSGGGGSGGGGSGGGGSGGGGSGGGGSGGGGSGGGGDKTHTCPPCPAPE

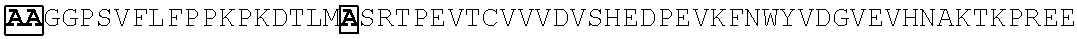

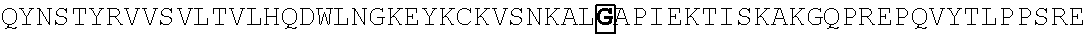

EMTKNQVSLTCLVKGFYPSDIAVEWESNGOPENNYKTTPPVLDSDGSFFLYSKLTVDKSRWQ
QGNVFSCSVMHEALHNHYTQKSLSLSPGKGGGGSGGGGSGGGGSGGGGSGGGGSGGSSQIRQ
NYSTDVEAAVNSLVNLYLQASYTYLSLGFYFDRDDVALEGVSHFFRELAEEKREGYERLLKM
QNQRGGRALFQDIKKPAEDEW
IgGI LALAP K288A scFc-N hFTL
SEQ ID NO:44

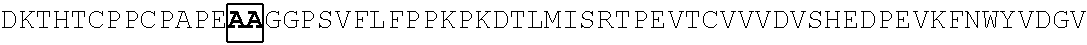

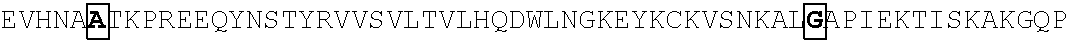

REPQVYTLPPSREEMTKNQVSLTCLVKGFYPSDIAVEWESNGQPENNYKTTPPVLDSDGSFF
LYSKLTVDKSRWQQGNVFSCSVMHEALHNHYTQKSLSLSPGKGGGGSGGGGSGGGGSGGGGS
GGGGSGGGGSGGGGSGGGGSGGGGSGGGGSGGGGSGGGGSGGGGSGGGGDKTHTCPPCPAPE

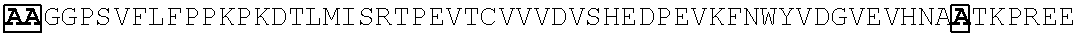

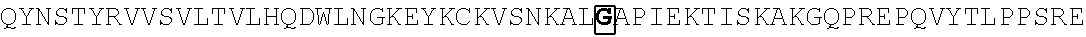

EMTKNQVSLTCLVKGFYPSDIAVEWESNGQPENNYKTTPPVLDSDGSFFLYSKLTVDKSRWQ
QGNVFSCSVMHEALHNHYTQKSLSLSPGKGGGGSGGGGSGGGGSGGGGSGGGGSGGSSQIRQ
NYSTDVEAAVNSLVNLYLQASYTYLSLGFYFDRDDVALEGVSHFFRELAEEKREGYERLLKM
QNQRGGRALFQDIKKPAEDEW
IgG2_P329G_K288A scFc-N hFTL
SEQ ID NO:45
VECPPCPAPPVAGPSVFLFPPKPKDTLMISRTPEVTCVVVDVSHEDPEVQFNWYVDGVEVHN

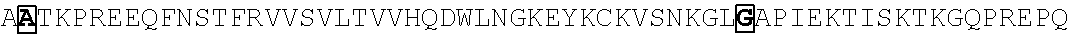

VYTLPPSREEMTKNQVSLTCLVKGFYPSDISVEWESNGQPENNYKTTPPMLDSDGSFFLYSK
LTVDKSRWQQGNVFSCSVMHEALHNHYTQKSLSLSPGKGGGGSGGGGSGGGGSGGGGSGGGG
SGGGGSGGGGSGGGGSGGGGSGGGGSGGGGSGGGGSGGGGSGGGGVECPPCPAPPVAGPSVF

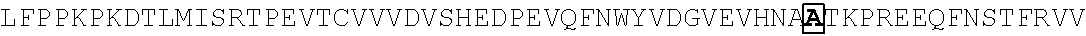

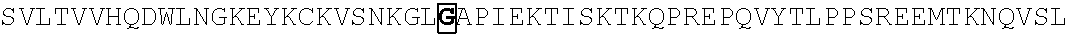

TCLVKGFYPSDISVEWESNGQPENNYKTTPPMLDSDGSFFLYSKLTVDKSRWQQGNVESCSV
MHEALHNHYTQKSLSLSPGKGGGGSGGGGSGGGGSGGGGSGGGGSGGSSQIRQNYSTDVEAA
VNSLVNLYLQASYTYLSLGFYFDRDDVALEGVSHFFRELAEEKREGYERLLKMQNORGGRAL
FQDIKKPAEDEW
IgG2 Fc
SEQ ID NO:46
VECPPCPAPPVAGPSVFLFPPKPKDTLMISRTPEVTCVVVDVSHEDPEVQFNWYVDGVEVHN
AKTKPREEQFNSTFRVVSVLTVVHQDWLNGKEYKCKVSNKGLPAPIEKTISKTKGQPREPQV
YTLPPSREEMTKNQVSLTCLVKGFYPSDISVEWESNGQPENNYKTTPPMLDSDGSFFLYSKL
TVDKSRWQQGNVFSCSVMHEALHNHYTQKSLSLSPGK
His tagged MPER peptide
SEQ ID NO:47
HHHHHHNEQELLELDKWASLWNWENITNWLWYIKKKK
Example GS repeat
SEQ ID NO:48
GGGS
Example GS repeat
SEQ ID NO:49
GGGGS

### EQUIVALENTS/OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

1. A self-assembled polypeptide complex comprising
(a) a plurality of first fusion polypeptides, each first fusion polypeptide comprising (1) an Fc polypeptide linked to (2) a nanocage monomer or subunit thereof, wherein the Fc polypeptide comprises a Fc chain having one or more mutations relative to a reference Fc chain of the same Ig class, and
(b) a plurality of second fusion polypeptides, each second fusion polypeptide comprising (1) an antigen-binding antibody fragment linked to (2) a nanocage monomer or subunit thereof.

2. The self-assembled polypeptide complex of claim 1,
wherein (1) if the Fc polypeptide is an IgG1 Fc polypeptide, the antigen-binding fragment is not a Fab fragment that binds to SARS-CoV-2; and/or (2) if the nanocage monomer is a mouse ferritin monomer and the Fc polypeptide is a mouse IgG2a Fc polypeptide, the antigen-binding antibody fragment is not a Fab fragment that binds to CD19.

3. The self-assembled polypeptide complex of claim 1, wherein the nanocage monomer is a ferritin monomer.

4. The self-assembled polypeptide complex of claim 3, wherein the ferritin monomer is a ferritin light chain.

5. The self-assembled polypeptide complex of claim 4, which does not comprise any ferritin heavy chains or subunits of ferritin heavy chains.

6. (canceled)

7. The self-assembled polypeptide complex of claim 1, wherein the Fc polypeptide is an IgG1 Fc polypeptide.

8. (canceled)

9. The self-assembled polypeptide complex of claim 1, wherein the Fc polypeptide is a single-chain Fc (scFc).

10. (canceled)

14. The self-assembled polypeptide complex of claim 1, wherein the one or more mutations comprise a mutation or set of mutations associated with altered binding to FcRn.

15. The self-assembled polypeptide complex of claim 14, wherein the mutation or set of mutations comprises a mutation at one or more of the following residues: M252, I253, S254, T256, K288, M428, and N434, or combinations thereof, wherein numbering is according to the EU index.

16. The self-assembled polypeptide complex of claim 15, wherein the mutation or set of mutations comprises mutations at M428 and N434, wherein numbering is according to the EU index.

17. The self-assembled polypeptide complex of claim 16, wherein the mutation or set of mutations comprises M428L and N434S mutations, wherein numbering is according to the EU index.

18. The self-assembled polypeptide complex of claim 14, wherein the altered binding to FcRn is decreased binding to FcRn.

19. The self-assembled polypeptide complex of claim 18, wherein the mutation or set of mutations associated with decreased binding to FcRn is selected from the group consisting of I253A, I253V, and K288A, and combinations thereof, wherein numbering is according to the EU index.

20. The self-assembled polypeptide complex of claim 1, wherein the one or more mutations comprise a mutation or set of mutations associated with altered effector function.

21. The self-assembled polypeptide complex of claim 20, wherein the Fc polypeptide is an IgG1 Fc polypeptide, and wherein the mutation or set of mutations comprises a mutation at one or more the following residues: L234, L235, G236, G237, P329, and A330, or combinations thereof, wherein numbering is according to the EU index.

22. The self-assembled polypeptide complex of claim 20, wherein the altered effector function is decreased effector function.

23. The self-assembled polypeptide complex of claim 22, wherein the mutation or set of mutations associated with decreased effector function is selected from the group consisting of LALA (L234A/L235A), LALAP (L234A/L235A/P329G), G236R, G237A, and A330L, wherein numbering is according to the EU index.

24.-53. (canceled)

54. A method comprising administering a composition comprising the self-assembled polypeptide complex of claim 1 to a mammalian subject.

55.-63. (canceled)

64. A fusion polypeptide comprising (1) an Fc polypeptide linked to (2) a nanocage monomer or subunit thereof, wherein the Fc polypeptide comprises a Fc chain having one or more mutations relative to a reference Fc chain of the same Ig class, wherein the one or more mutations comprise a mutation or set of mutations associated with altered binding to FcRn and/or with altered effector function.

65. The fusion polypeptide of claim 64, wherein the nanocage monomer is a ferritin monomer.

66. The fusion polypeptide of claim 65, wherein the ferritin monomer is a ferritin light chain.

67. The fusion polypeptide of claim 66, which does not comprise any ferritin heavy chains or subunits of ferritin heavy chains.

68. The fusion polypeptide of claim 65, wherein the ferritin monomer is a human ferritin.

69. The fusion polypeptide of claim 64, wherein the Fc polypeptide is an IgG1 Fc polypeptide.

70. (canceled)

71. The fusion polypeptide of claim 64, wherein the Fc polypeptide is a scFc.

72. The fusion polypeptide of claim 6, wherein the mutation or set of mutations comprises a mutation at one or more of the following residues: M252, I253, S254, T256, K288, M428, and N434, or combinations thereof, wherein numbering is according to the EU index.

73. The fusion polypeptide of claim 64, wherein the altered binding to FcRn is decreased binding to FcRn.

74. The fusion polypeptide of claim 73, wherein the mutation or set of mutations associated with decreased binding to FcRn is selected from the group consisting of I253A, I253V, and K288A, and combinations thereof, wherein numbering is according to the EU index.

75. The fusion polypeptide of claim 64, wherein the Fc polypeptide is an IgG1 Fc polypeptide, and wherein the mutation or set of mutations comprises a mutation at one or more the following residues: L234, L235, G236, G237, P329, and A330, or combinations thereof, wherein numbering is according to the EU index.

76. The fusion polypeptide of claim 64, wherein the altered effector function is decreased effector function.

77. The fusion polypeptide of claim 76, wherein the mutation or set of mutations associated with decreased effector function is selected from the group consisting of LALA (L234A/L235A), LALAP (L234A/L235A/P329G), G236R, G237A, and A330L, wherein numbering is according to the EU index.

78.-80. (canceled)


SEQUENCE LISTING (49 sequences)

SEQ ID NO: 1 - protein, 175 aa, synthetic construct
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 2 - protein, 90 aa, synthetic construct
MSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 3 - protein, 85 aa, synthetic construct
GKTPDAMKAAMALEKKLNQALLDLHALGSARTDPHLCDFLETHFLDEEVKLIKKMGDHLTNLHRLGGPEAGLGEYLFERLTLRHD

SEQ ID NO: 4 - protein, 85 aa, synthetic construct
GKTPDAMKAAMALEKKLNQALLDLHALGSARTDPHLCDFLETHFLDEEVKLIKKMGDHLTNLHRLGGPEAGLGEYLFERLTLKHD

SEQ ID NO: 5 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 523 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 226 aa, synthetic construct
KPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 8 - protein, 515 aa, synthetic construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 9 - protein, 227 aa, synthetic construct
KYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 523 aa, synthetic construct
KYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGGGGSGGGGSGGGGSGGGGSGGGGSGGGGSGGGGSGGGGSGGGGSGGGGKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 471 aa, synthetic construct
QAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 219 aa, synthetic construct
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 244 aa, synthetic construct
QAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 14 - protein, 533 aa, synthetic construct
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 15 - protein, 457 aa, synthetic construct
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 204 aa, synthetic construct
QSALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 17 - protein, 230 aa, synthetic construct
ADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 504 aa, synthetic construct
QSALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 19 - protein, 461 aa, synthetic construct
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 234 aa, synthetic construct
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 22 - protein, 517 aa, synthetic construct
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 23 - protein, 452 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 219 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 225 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYC

SEQ ID NO: 26 - protein, 514 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 27 - protein, 735 aa, synthetic construct
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 28 - protein, 616 aa, synthetic construct
QSALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 29 - protein, 620 aa, synthetic construct
QSALTQPRSVSASPGQSVTISCTGTHNLVSWCQHQPGRAPKLLIYDFNKRPSGVPDRFSGSGSGGTASLTITGLQDDDDAEYFCWAYEAFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSADLVQSGAVVKKPGDSVRISCEAQGYRFPDYIIHWIRRAPGQGPEWMGWMNPMGGQVNIPWKFQGRVSMTRDTSIETAFLDLRGLKSDDTAVYYCVRDRSNGSGKRFESSNWFLDLWGRGTAVTIQSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 30 - protein, 630 aa, synthetic construct
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 31 - protein, 627 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSGGGGSGGGGSQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 32 - protein, 883 aa, synthetic construct
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGGKTPDAMKAAMALEKKLNQALLDLHALGSARTDPHLCDFLETHFLDEEVKLIKKMGDHLTNLHRLGGPEAGLGEYLFERLTLRHDGGSGGSGGSGGSGGGSGGSGGSGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 883 aa, synthetic construct
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECGGGGSGGGGSGGGGSGGGGSGGGGSGGGGSGGGGSGGGGSGGGGSGGGGSGGGGSGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSGGGGSGGGGKTPDAMKAAMALEKKLNQALLDLHALGSARTDPHLCDFLETHFLDEEVKLIKKMGDHLTNLHRLGGPEAGLGEYLFERLTLRHDGGSGGSGGSGGSGGGSGGSGGSGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 34 - protein, 639 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 35 - protein, 631 aa, synthetic construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 36 - protein, 639 aa, synthetic construct
KYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGGGGSGGGGSGGGGSGGGGSGGGGSGGGGSGGGGSGGGGSGGGGSGGGGKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 37 - protein, 639 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 38 - protein, 639 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 39 - protein, 639 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 40 - protein, 639 aa, synthetic construct
DKTHTCPPCPAPELLGGPSVFLFPPKPKDTLMISRTPEVTCVVVDVSHEDPEVKFNWYVDGVEVHNAATKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGDKTHTCPPCPAPELLGGPSVFLFPPKPKDTLMISRTPEVTCVVVDVSHEDPEVKFNWYVDGVEVHNAATKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 41 - protein, 639 aa, synthetic construct
DKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGDKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 42 - protein, 639 aa, synthetic construct
DKTHTCPPCPAPELLGGPSVFLFPPKPKDTLMVSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGDKTHTCPPCPAPELLGGPSVFLFPPKPKDTLMVSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 43 - protein, 639 aa, synthetic construct
DKTHTCPPCPAPEAAGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGDKTHTCPPCPAPEAAGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 44 - protein, 639 aa, synthetic construct
DKTHTCPPCPAPEAAGGPSVFLFPPKPKDTLMISRTPEVTCVVVDVSHEDPEVKFNWYVDGVEVHNAATKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGDKTHTCPPCPAPEAAGGPSVFLFPPKPKDTLMISRTPEVTCVVVDVSHEDPEVKFNWYVDGVEVHNAATKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 45 - protein, 631 aa, synthetic construct
VECPPCPAPPVAGPSVFLFPPKPKDTLMISRTPEVTCVVVDVSHEDPEVQFNWYVDGVEVHNAATKPREEQFNSTFRVVSVLTVVHQDWLNGKEYKCKVSNKGLGAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGVECPPCPAPPVAGPSVFLFPPKPKDTLMISRTPEVTCVVVDVSHEDPEVQFNWYVDGVEVHNAATKPREEQFNSTFRVVSVLTVVHQDWLNGKEYKCKVSNKGLGAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGKGGGGSGGGGSGGGGSGGGGSGGGGSGGSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 46 - protein, 223 aa, synthetic construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 47 - protein, 37 aa, synthetic construct
HHHHHHNEQELLELDKWASLWNWFNITNWLWYIKKKK

SEQ ID NO: 48 - protein, 4 aa, synthetic construct
GGGS

SEQ ID NO: 49 - protein, 5 aa, synthetic construct
GGGGS